(19) United States
(12) Patent Application Publication
(10) Pub. No.: US 2022/0339265 A1
(43) Pub. Date: Oct. 27, 2022

(54) MODULATION OF UBIQUITIN CARBOXY-TERMINAL HYDROLASE LIGASE 1 (UCHL1) EXPRESSION FOR TREATING NEUROLOGICAL DISEASE, DISORDERS, AND INJURIES ASSOCIATED WITH UPPER MOTOR NEURONS

(71) Applicant: Northwestern University, Evanston, IL (US)

(72) Inventors: P. Hande Ozdinler, Chicago, IL (US); Javier H. Jara, Chicago, IL (US); Baris Genc, Evanston, IL (US)

(21) Appl. No.: 17/838,428

(22) Filed: Jun. 13, 2022

Related U.S. Application Data
(63) Continuation-in-part of application No. PCT/US2020/064939, filed on Dec. 14, 2020.
(60) Provisional application No. 62/947,189, filed on Dec. 12, 2019.

(51) Int. Cl. A61K 38/47; A61K 48/00; A61P 25/28

(52) U.S. Cl. CPC A61K 38/47; A61K 48/0008; A61K 48/0066; A61P 25/28

(57) ABSTRACT

Disclosed are compositions and methods for treating neurological diseases, disorders, and injuries in a subject in need thereof. Particularly disclosed are compositions and methods for treating neurological diseases, disorders, and injuries that are associated with upper motor neurons in a subject in need thereof in which methods expression of ubiquitin carboxyl hydrolase ligase 1 (UCHL1) is modulated in the subject, for example, via gene therapy being administered to the subject in order to express UCHL1 in upper motor neurons of the subject. Also disclosed are expression vectors comprising the UCHL1 promoter operably linked to a nucleic acid encoding a therapeutic gene product.

### CROSS-REFERENCE TO RELATED APPLICATIONS

This application is a Continuation-in-Part of PCT/US2020/064939 with international filing date of Dec. 14, 2020, and which claims the benefit of U.S. Provisional Application 62/947,189, filed Dec. 12, 2019. The entire content of each of the above-referenced applications is incorporated herein by reference.

### STATEMENT REGARDING FEDERALLY SPONSORED RESEARCH OR DEVELOPMENT

This invention was made with government support under grant NS085161 awarded by the National Institutes of Health. The government has certain rights in the invention.

### SEQUENCE LISTING

A Sequence Listing accompanies this application and is submitted as an ASCII text file of the sequence listing named “702581_02176_ST25.txt” which is 39,551 bytes in size and was created on Jun. 13, 2022. The sequence listing is electronically submitted via EFS-Web with the application and is incorporated herein by reference in its entirety.

### FIELD

The field of the invention relates to compositions and methods for treating neurological diseases, disorders, and injuries in a subject in need thereof. In particular, the field of the invention relates to compositions and methods for treating neurological diseases, disorders, and injuries that are associated with upper motor neurons in a subject in need thereof in which methods expression of ubiquitin carboxyl hydrolase ligase 1 (UCHL1) is modulated in the subject, for example, via gene therapy being administered to the subject in order to express UCHL1 in upper motor neurons of the subject.

### BACKGROUND

There is an imperative need to establish novel methods to deliver genes of interest into relevant neuron populations within the cerebral cortex, without affecting other neurons and cells. Upper motor neurons (UMN) die in patients suffering from diseases in which voluntary movement is impaired, such as amyotrophic lateral sclerosis (ALS), hereditary spastic paraplegia (HSP), primary lateral sclerosis (PLS) as well as patients with spinal cord injury. Therefore, improving UMN health has great clinical importance. Here, we disclose a method of administering gene delivery to UMNs by using the right combination of promoter and adenovirus-associated virus (AAV) serotype, and disclose the importance of the TH ubiquitin carboxyl hydrolase ligase 1 (UCHL1) promoter for enhanced specificity. We also disclose that the UCHL1 protein is very important for promoting the health of UMNs. Adeno-associated viruses (AAV) are used for gene delivery approaches in clinical trials and some are FDA approved. We have previously demonstrated the feasibility of AAV specific-delivery properties in the cerebral cortex of mouse models and are currently assessing their utilization in non-human primates. We disclose the use of the UCHL1 promoter to drive gene expression primarily in UMNs and to develop directed gene delivery approaches to UMNs with respect to diseases and injury. The extent of UMN transduction can be modulated by the choice of promoter and these can be developed based on the patient's needs, allowing for personalized medicine. Because UMNs are clinically important, their selective genetic modulation will offer therapeutic interventions.

### SUMMARY

Disclosed are compositions and methods for treating neurological diseases, disorders, and injuries in a subject in need thereof. Particularly disclosed are compositions and methods for treating neurological diseases, disorders, and injuries that are associated with upper motor neurons in a subject in need thereof in which methods expression of ubiquitin carboxyl hydrolase ligase 1 (UCHL1) is modulated in the subject, for example, via gene therapy being administered to the subject in order to express UCHL1 in upper motor neurons of the subject.

In some aspects, the subject has a neurodegenerative disease or disorder selected from amyotrophic lateral sclerosis (ALS), hereditary spastic paraplegia (HSP), primary lateral sclerosis (PLS), or a spinal cord injury.

In some aspects, the therapeutic agent is administered to the subject and comprises a vector that expresses UCHL1 or a variant thereof comprising an amino acid sequence of SEQ ID NO 1-6, or a variant thereof, or a variant having at least about 80% sequence identity to any of SEQ ID NOs:1-6, or a combination thereof. In some aspects, the vector comprise a viral vector, such as an adenovirus-associated viral (AAV) vector serotype 1, 2, 3, 4, 5, 6, 7, 8, or 9.

In some aspects, the vector comprises a promoter, selected from CMV promoter (i.e., strong mammalian expression promoter from the human cytomegalovirus), CBA promoter (i.e., chicken β-actin promoter), UCHL1 promoter (i.e., promoter for ubiquitin carboxy-terminal hydrolase ligase 1 gene), EF1α promoter (i.e., strong mammalian expression from human elongation factor 1α), SV40 promoter (i.e., mammalian expression promoter from the simian vacuolating virus 40), PGK1 promoter (i.e., mammalian promoter from phosphoglycerate kinase gene), Ubc promoter (i.e., mammalian promoter from the human ubiquitin C gene), and human beta actin promoter (i.e., mammalian promoter from beta actin gene). In some embodiments, the vector expresses UCHL1 (e.g., any one or a combination of SEQ ID Nos: 1-6, or variants thereof).

Also disclosed herein are expression vectors comprising the UCHL1 promoter. In some embodiments, the vector comprises a viral vector, such as an AAV viral vector. In some embodiments, the UCHL1 promoter is operably linked to a nucleic acid sequence encoding a therapeutic molecule, such as a polypeptide or a nucleic acid (e.g., RNA, such as siRNA).

Also disclosed herein are pharmaceutical compositions comprising one or more of the above-described vectors; and a carrier, excipient, or diluent.

### BRIEF DESCRIPTION OF THE FIGURES

FIG. 1A-H: UCHL1 expression can be deleted using cre/lox conditional mutant approach in the CSMN of floxed UCHL1 mice. (A) Floxed UCHL1 (UCHL1^{f/f}) mice were generated by introducing loxP sites flanking the exon 4 of UCHL1. Cre recombinase activity leads to removal of the exon 4. (B) Deletion of exon 4 of mouse UCHL1 gene introduces a de novo stop codon shortly after the deletion site. (C) Rbp4^{cre} mice drive the expression of cre recombinase in layer 5 subcortical projection neurons including the CSMN. HB9^{cre} mice drive the expression of cre recombinase in spinal motor neurons located in the ventral horn of the spinal cord. Crossing these cre driver mice with UCHL1^{f/f} mice leads to conditional mutant mice that lack UCHL1 function either in the CSMN or the SMN. (D-H) Representative images of primary motor cortex from UCHL1^{f/f}(D-E), Rbp4^{cre} UCHL1^{f/f} (F-G), and HB9^{cre} UCHL1^{f/f} (H) mice at P30 (D, F) and P100 (E, G, H). CSMN can be identified by Ctip2⁺ nuclei. Scale bars on top panel=250 μm (D′-H′) Representative images of layer 5 of primary motor cortex from UCHL1^{f/f} (D′-E′), Rbp4^{cre} UCHL1^{f/f} (F′-G′), and HB9^{cre} UCHL1^{f/f} (H′) mice at P30 (D′, F′) and P100 (E′, G′, H′). Scale bar=50 μm. (D″-H″) Representative images of CSMN from UCHL1^{f/f} (D″-E″), Rbp4^{cre} UCHL1^{f/f} (F″-G″), and HB9^{cre} UCHL1^{f/f} (H″) mice at P30 (D″, F″) and P100 (E″, G″, H″) captured by confocal microscope. Scale bar=10 μm.

FIG. 2A-E: HB9^{cre} UCHL1^{f/f} mice lack UCHL1 in their spinal motor neurons (SMN) (A-E) Representative images of the ventral horn of the spinal cord of UCHL1^{f/f} (A-B), HB9^{cre} UCHL1^{f/f} (C-D), and Rbp4^{cre} UCHL1^{f/f} (E) mice at P30 (A, C) and P100 (B, D, E). Scale bar=100 μm. (A′-E′) Representative images of ChAT⁺ SMN from UCHL1^{f/f} (A′-B′), HB9^{cre} UCHL1^{f/f} (C′-D′), and Rbp4^{cre} UCHL1^{f/f} (E′) mice at P30 (A′, C′) and P100 (B′, D′, E′) captured by confocal microscope. Scale bar=20 μm.

FIG. 3A-F: Cortex specific UCHL1 conditional mutant mice recapitulate pathology observed in the CSMN of UCHL1^{-/-} mice. (A) Schematic overview of the experimental setup. CSMN of UCHL1^{f/f}, UCHL1^{-/-}, Rbp4^{cre} UCHL1^{f/f}, and HB9^{cre} UCHL1^{f/f} mice were retrogradely transduced by injecting AAV2-eGFP into the corticospinal tract (CST) at P30, and CSMN were analyzed at P100. (B) Representative images of GFP labeled CSMN after DAB mediated immunohistochemistry enhancement of the GFP signal allowing investigation of CSMN morphology in detail. Scale bar=20 μm. (C) Primary apical dendrites of CSMN retrogradely labeled by AAV2-eGFP. Scale bar=10 μm. (D) Cell bodies of CSMN retrogradely labeled by AAV2-eGFP. Scale bar=20 μm. (E) Quantitative analysis of apical dendrites reveals significant increase in the average percentage of apical dendrites with vacuoles in the absence of UCHL1. Data presented as mean±SEM; *p<0.05; **p<0.01; ***p<0.001; one-way ANOVA with post hoc Tukey's multiple comparison test. (F) Quantitative analysis of soma size shows significant reduction in the average CSMN diameter in the absence of UCHL1. Data presented as mean±SEM; **p<0.01; ***p<0.001; one-way ANOVA with post hoc Tukey's multiple comparison test.

FIG. 4A-C: Spine density is reduced in degenerating and vacuolated apical dendrites of CSMN. (A) Representative images of healthy and vacuolated CSMN primary apical dendrites at P100. Scale bar=10 μm (B) Quantitative analysis of average spine density along the primary apical dendrites of healthy CSMN. Data presented as mean±SEM. (C) Quantitative analysis of average spine density along primary apical dendrites of CSMN with vacuoles. Data presented as mean±SEM.

FIG. 5A-D: UCHL1 expression can be restored in CSMN of UCHL1-mice using a AAV2-eGFP-IRES-UCHL1 bicistronic expression vector. (A) Schematic overview of the experimental setup. CSMN of wild type (WT), or UCHL1^{-/-} mice were retrogradely transduced by injecting AAV2-eGFP into the corticospinal tract (CST) at P30, and CSMN were analyzed at P100. AAV2-eGFP-IRES-UCHL1 virus was used for retrograde transduction of CSMN with UCHL1 expression in the UCHL1^{-/-} mice. (B-D) Representative images of CSMN retrogradely transduced by AAV2-eGFP in WT (B), UCHL1^{-/-} (C) and AAV2-eGFP-IRES-UCHL1 in UCHL1^{-/-} (D) mice. Scale bar=20 μm.

FIG. 6A-D: Selective and directed UCHL1 expression in only CSMN is sufficient to restore the integrity of apical dendrites in the UCHL1^{-/-} mice. (A) Schematic overview of the experimental setup. CSMN of wild type (WT), or UCHL1^{-/-} mice were retrogradely transduced by injecting AAV2-eGFP into the corticospinal tract (CST) at P30, and CSMN apical dendrites were analyzed at P100. AAV2-eGFP-IRES-UCHL1 virus was used for retrograde transduction of CSMN with UCHL1 expression in UCHL1^{-/-} mice. (B-D) Representative images of CSMN apical dendrites retrogradely transduced by AAV2-eGFP in WT (B), UCHL1^{-/-} (C) and AAV2-eGFP-IRES-UCHL1 in UCHL1^{-/-} (D) mice. Scale bar=10 μm.

FIG. 7A-F: Restoration of UCHL1 expression in only CSMN of UCHL1^{-/-} mice is sufficient to improve their health and cytoarchitectural integrity. (A) Schematic overview of the experimental setup. CSMN of wild type (WT), or UCHL1^{-/-} mice were retrogradely transduced by injecting AAV2-eGFP into the corticospinal tract (CST) at P30, and CSMN apical dendrites were analyzed at P100. AAV2-eGFP-IRES-UCHL1 virus was used to rescue UCHL1 expression in UCHL1^{-/-} mice. (B) Representative images of GFP labeled CSMN after DAB mediated immunohistochemistry enhancement of the GFP signal allowing investigation of CSMN morphology in detail. Scale bar=20 μm. (C) Primary apical dendrites of CSMN retrogradely labeled by AAV2-eGFP or AAV2-eGFP-IRES-UCHL1. Scale bar=10 μm. (D) Cell bodies of CSMN retrogradely labeled by AAV2-eGFP or AAV2-eGFP-IRES-UCHL1. Scale bar=20 μm. (E) Quantitative analysis of apical dendrites reveals significant decrease in the average percentage of apical dendrites with vacuoles when UCHL1 expression is restored to the UCHL1^{-/-} CSMN. Data presented as mean±SEM; ****p<0.0001; one-way ANOVA with post hoc Tukey's multiple comparison test. (F) Quantitative analysis of soma size shows significant increase in the average CSMN diameter when UCHL1 expression is restored to the UCHL1^{-/-} CSMN. Data presented as mean±SEM; **p<0.01; one-way ANOVA with post hoc Tukey's multiple comparison test.

FIG. 8A-O: hUCHL1 promoter driven gene expression selectively transduces upper motor neurons in Macaque Monkeys. (A) AAV2, which encodes a plasmid that drives mCherry expression under the control of human UCHL1 (hUCHL1) promoter, was injected into one side of the Broadmann's area 4 of the motor cortex in Macaque monkeys (n=2). (B) Motor cortex was sectioned and analyzed. Large Betz cells located in layer V of motor cortex were transduced in Monkey #1 (C) and in Monkey #2 (D). (E) The contralateral side of the cortex had no immunoreactivity. (F) Enlarged images taken from layer V of the motor cortex confirmed the identity of transduced neurons to be the upper motor neurons. (G-K) Brainstem was sectioned and studied to further investigate whether transduced neurons project to subcerebral cortex. (G) The magnification image of the medullary pyramids within the brainstem. The injected site has a high density of axonal fibers (G), which are better visualized with increased magnification (H, and J). The contralateral side of the medullary pyramids is devoid of labeled axonal fibers. (L-O) Spinal cord was sectioned and further investigated to visualize corticospinal tract axons and their connections with the spinal motor neurons. (L) The ventral horn of the spinal cord is where the spinal motor neurons reside and numerous axon fibers entering the ventral horn were detected. (M) Numerous examples of upper motor neuron axon fibers located in close proximity to spinal motor neurons were noted. (N) The cross section of the anterior corticospinal tract within the spinal cord display dense axon fibers crossing into the spinal cord. The contralateral side is does not have axon fibers. (O) Detailed investigation of axon fibers also reveal direct connections of the upper motor neuron axons with spinal motor neurons. In some cases, the growth cones and the site of connectivity was visualized, further confirming the identity of neurons that are transduced to be upper motor neurons.

FIG. 9A-K: Expression of UCHL1 selectively in upper motor neurons that become diseased due to mutant SOD1 toxicity and TDP-43 pathology, improve their health and reverse them back to the WT healthy control levels. Corticospinal motor neurons (CSMN, the upper motor neurons in mice) were retrogradely transduced by AAV2. The AAV construct expresses UCHL1 gene, so that retrogradely transduced CSMN expressed UCHL1. eGFP was used as a marker to label cells for visualization. The control plasmid has only the eGFP gene. CSMN were transduced in WT mice (A), in hSOD1G93A mice (B), and in TDP-43A315T mice (D). UCHL1 was expressed in CSMN that are diseased due to mutant SOD1 toxicity (C), and in CSMN that are diseased due to TDP-43 pathology (E). (F) Healthy CSMN have large soma and a prominent apical dendrite, whereas diseased CSMN have smaller soma and apical dendrite that is disintegrating with numerous vacuoles. (G) A closer look at the apical dendrite reveals that the healthy CSMN have prominent apical dendrite, whereas apical dendrites of diseased CSMN in both hSOD1G93A and TDP-43A3145T mice have vacuolated apical dendrites. (H) Bar graph representation of the quantitation performed on CSMN of WT mice transduced with control virus (n=3), with virus expressing UCHL1 (n=3), CSMN of hSOD1G93A mice transduced with control virus (n=4), with virus expressing UCHL1 (n=7) to reveal the soma diameter. Expression of UCHL1 in CSMN of hSOD1G93A mice significantly increases the soma diameter. (I) Bar graph representation of the quantitation performed on CSMN of WT mice transduced with control virus (n=3), with virus expressing UCHL1 (n=3), CSMN of hSOD1G93A mice transduced with control virus (n=4), with virus expressing UCHL1 (n=7) to reveal the percent apical dendrites with vacuoles. Expression of UCHL1 in CSMN of hSOD1G93A mice significantly reduces vacuolization of apical dendrites. Similar experiments are performed in the TDP-43A315T mice (D, E, J,K). (J) Bar graph representation of the quantitation performed on CSMN of WT mice transduced with control virus (n=3), with virus expressing UCHL1 (n=3), CSMN of TDP-43A315T mice transduced with control virus (n=4), with virus expressing UCHL1 (n=4) to reveal the soma diameter. Expression of UCHL1 in CSMN of TDP-43A315T mice significantly increases the soma diameter. (K) Bar graph representation of the quantitation performed on CSMN of WT mice transduced with control virus (n=3), with virus expressing UCHL1 (n=3), CSMN of TDP-43A315T mice transduced with control virus (n=4), with virus expressing UCHL1 (n=4) to reveal the percent apical dendrites with vacuoles. Expression of UCHL1 in CSMN of TDP-43A315T mice significantly reduces vacuolization of apical dendrites. Data presented as mean±SEM; **=p<0.005; ***=p<0.001; ***=p<0.0001; one-way ANOVA with post hoc Tukey's multiple comparison test.

FIG. 10A-D: AAV2-UCHL1-mCherry transduction of motor cortex labels subcerebral projection neurons, including CSMN, as evidenced by expression in the soma and in the apical dendrites in mice. (A) Schematic drawing of a coronal section of a UCHL1-eGFP mouse and the site of AAV2-UCHL1-mCherry injection into the motor cortex. (B) Representative image of the coronal section of UCHL1-eGFP mouse brain, which includes the site of injection. Corticospinal motor neurons (CSMN) are labeled with eGFP in the UCHL1-eGFP mice and they are located in layer V of the motor cortex. mCherry expressing neurons, which are cells transduced by AAV2-UCHL1-mCherry, are mainly located in layer V and layer VI of the motor column. There is no mCherry expression at top layers of the cortex and AAV-transduction is restricted to the deep layers of the motor cortex, which harbor subcerebral projection neurons that degenerate in ALS and related motor neuron diseases. (C) Representative images of cell bodies obtained from two different sets of experiments. Green and red channels are separated to the side. Green cells are CSMN (a.k.a. upper motor neurons) and red cells are the ones transduced with AAV-UCHL1-mCherry. The co-localization of red and green cells especially in Layer 5 of the motor cortex is evident. Not all CSMN are transduced, but most of the cells that are transduces are indeed CSMN. The use of UCHL1 promoter enhances transduction efficiency of CSMN. None of the cells that are transduced are astrocytes or microglia. (D) Representative image of apical dendrites that extent towards the top layers of the brain. CSMN have long apical dendrites and they are eGFP+. Likewise, transduced CSMN have mCherry+ apical dendrites that colocalize, further proving that transduction targets CSMN. Not all CSMN express mCherry, but almost all mCherry+ cells are CSMN (eGFP+), further proving that upon one time injection to the motor cortex, transduction is restricted to deep layers of the motor cortex and is more selective to CSMN in layer V.

FIG. 11A-C: AAV2-UCHL1-mCherry transduction of motor cortex labels subcerebral projection neurons, including CSMN, in mice, as evidenced by their axonal projection field. (A) Schematic drawing of a coronal section of a mouse brain and the site of AAV2-UCHL1-mCherry injection into the motor cortex of UCHL1-eGFP mice. mCherry is expressed under the control of UCHL1 promoter and AAV2 serotype is used for AAV-mediated transduction. In UCHL1-eGFP mice corticospinal motor neurons (CSMN) are genetically labeled by eGFP expression. (B) A representative image of striatum at the site of injection. Axonal projections of subcerebral projection neurons, including CSMN (a.k.a. upper motor neurons), are labeled with green fluorescence in the UCHL1-eGFP mice and they are perfectly co-localized by mCherry expression. A selected area is enlarged below. (C) A representative image of striatum at the contralateral site of injection. Axonal projections of subcerebral projection neurons, including CSMN (a.k.a. upper motor neurons), are labeled with green fluorescence in the UCHL1-eGFP mice, but they do not have any mCherry expression.

### DETAILED DESCRIPTION

The present invention is described herein using several definitions, as set forth below and throughout the application.

Unless otherwise specified or indicated by context, the terms “a”, “an”, and “the” mean “one or more.” For example, “a therapy” should be interpreted to mean “one or more therapies.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “subject” may be used interchangeably with the terms “patient” or “individual” and means an animal, which may be a human or non-human animal, in need of treatment. A “subject in need thereof” may include a subject having a disease or disorder associated with the activity of upper motor neurons. A “subject in need thereof” may include a subject having a neurological disease, disorder, or injury, such as a subject having a neurological disease, disorder, or injury associated with the activity of upper motor neurons. A “subject in need thereof” may include, but is not limited to, a subject having or at risk for developing amyotrophic lateral sclerosis (ALS), hereditary spastic paraplegia (HSP), or primary lateral sclerosis (PLS). A “subject in need thereof” may include, but is not limited to, a subject having or at risk for developing a spinal cord injury.

UCHL1

Reference is made herein to ubiquitin carboxy-terminal hydrolase ligase 1 (HCHL1), also referred to as ubiquitin carboxyl-terminal esterase L1, ubiquitin thiolesterase, ubiquitin carboxyl-terminal hydrolase isozyme L1, neuron cytoplasmic protein, ubiquitin thioesterase, and ubiquitin thiolesterase, and variants thereof. Human UCHL1 (Homo sapiens) exists as at least five (5) isoforms, the amino acid sequences of which are provided herein as SEQ ID NOs:1-5, respectively.

SEQ ID NO: 1

| | |
|---|---|
| 1 | mqlkpmeinp emlnkvlsrl gvagqwrfvd vlgleeeslg |
| | svpapacall llfpltaqhe |
| 61 | nfrkkqieel kgqevspkvy fmkqtignsc gtiglihava |
| | nnqdklgfed gsvlkqflse |
| 121 | tekmspedra kcfekneaiq aandavaqeg qcrvddkvnf |
| | hfilfnnvdg hlyeldgrmp |
| 181 | fpvnhgasse dtllkdaakv creftereqg evrfsavalc |
| | kaa |

SEQ ID NO: 2

| | |
|---|---|
| 1 | mkqtignscg tiglihavan nqdklgfedg svlkqflset |
| | ekmspedrak cfekneaiqa |
| 61 | ahdavaqegq crvddkvnfh filfnnvdgh lyeldgrmpf |
| | pvnhgassed tllkdaakvc |
| 121 | reftereqge vrfsavalck aa |

SEQ ID NO: 3

| | |
|---|---|
| 1 | mkqtignscg tiglihavan nqdklgfedg svlkqflset |
| | ekmspedrak cfeknevddk |
| 61 | vnfhfilfnn vdghlyeldg rmpfpvnhga ssedtllkda |
| | akvcrefter eqgevrfsav |
| 121 | alckaa |

SEQ ID NO: 4

| | |
|---|---|
| 1 | mqlkpmeinp evsvlsrlgv agqwrfvdvl gleeeslgsv |
| | papacallll fpltaqhenf |
| 61 | rkkqieelkg qevspkvyfm kqtignscgt iglihavann |
| | qdklgfedgs vlkqflsete |
| 121 | kmspedrakc fekneaiqaa hdavaqegqc rvddkvnfhf |
| | ilfnnvdghl yeldgrmpfp |
| 181 | vnhgassedt llkdaakvcr eftereqgev rfsavalcka |
| | a |

SEQ ID NO: 5

| | |
|---|---|
| 1 | gsascfsssl gyfcralred aaqadgdqpr daeqsewrla |
| | pslapspasa eagaptgsgc |
| 61 | wqgpsrplra petaglgrgl gafpgplhla gdstkpvtgr |
| | rrglrpvase rqaelprawr |
| 121 | gqhrlgctgf agatcgprfv lchcagpggg graglgllpg |
| | sgagaegarl lapppgrcpr |
| 181 | pacprapgrl vsspqvlsrl gvagqwrfvd vlgleeeslg |
| | svpapacall llfpltaqhe |
| 241 | nfrkkqieel kgqevspkvy fmkqtignsc gtiglihava |
| | nnqdklgfed gsvlkqflse |
| 301 | tekmspedra kcfekneaiq aandavaqeg qcrvddkvnf |
| | hfilfnnvdg hlyeldgrmp |
| 361 | fpvnhgasse dtllkdaakv creftereqg evrfsavalc |
| | kaa |

The amino acid sequence of mouse (Mus musculus) UCHL1 is provided herein as SEQ ID NO:6.

SEQ ID NO: 6

| | |
|---|---|
| 1 | mqlkpmeinp emlnkvlakl gvagqwrfad vlgleeetlg |
| | svpspacall llfpltaqhe |
| 61 | nfrkkqieel kgqevspkvy fmkqtignsc gtiglihava |
| | nnqdklefed gsvlkqflse |
| 121 | teklspedra kcfekneaiq aandsvaqeg qcrvddkvnf |
| | hfilfnnvdg hlyeldgrmp |
| 181 | fpvnhgasse dsllqdaakv creftereqg evrfsavalc |
| | kaa |

As contemplated herein, UCHL1 or a variant thereof may comprise the amino acid sequence of any of SEQ ID NOs:1-6, or may comprises an amino acid sequence having at least about 50%, 60%, 70%, 80%, 90%, 95%, 96%, 97%, 98%, or 99% sequence identity to any of SEQ ID NOs:1-6. Variants of UCHL1 may include polypeptides having one or more amino acid substitutions, deletions, additions and/or amino acid insertions relative to a UCHL1 reference polypeptide (e.g., relative to any of SEQ ID NOs:1-6), where optionally the variant of UCHL1 exhibits ubiquitin carboxyl hydrolase ligase 1 (UCHL1) activity. Also disclosed are nucleic acid molecules that encode UCHL1 or a variant thereof (e.g., polynucleotides that encode the polypeptide of SEQ ID NOs:1 or 2 or variants thereof).

The disclosed UCHL1 polypeptides or variant polypeptides optionally exhibit one or more biological activities associated with wild-type UCHL1. For example, the disclosed UCHL1 polypeptides or variant polypeptides may add ubiquitin to a substrate and/or may remove ubiquitin from a substrate and/or regulate protein degradation and/or turnover of the substrate.

As used herein, the terms “amino acid” and “amino acid sequence” refer to an oligopeptide, peptide, polypeptide, or protein sequence (which terms may be used interchangeably), or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The amino acid sequences contemplated herein may include conservative amino acid substitutions and/or non-conservative amino acid substitutions relative to a reference amino acid sequence. For example, a variant UCHL1 polypeptide may include conservative amino acid substitutions and/or non-conservative amino acid substitutions relative to the wild-type UCHL1 polypeptide. “Conservative amino acid substitutions” are those substitutions that are predicted to interfere least with the properties of the reference polypeptide. In other words, conservative amino acid substitutions substantially conserve the structure and the function of the reference protein. The following table provides a list of exemplary conservative amino acid substitutions.

| Original | Conservative |
|---|---|
| Residue | Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asn, Glu, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

In contrast, “Non-conservative amino acid substitutions” are those substitutions that are predicted to interfere most with the properties of the reference polypeptide. For example, non-conservative amino acid substitutions may not conserve the structure and/or the function of the reference protein (e.g., substitution of a polar amino acid for a non-polar amino acid and/or substitution of a negatively charged amino acid for a positively charged amino acid).

A “deletion” refers to a change in the amino acid or nucleotide sequence that results in the absence of one or more amino acid residues or nucleotides relative to a reference sequence (e.g. relative to the amino acid sequence of any of SEQ ID NOs:1-6). A deletion may remove at least 1, 2, 3, 4, 5, 10, 20, 50, 100, or 200 amino acids residues or nucleotides. A deletion may include an internal deletion or a terminal deletion (e.g., an N-terminal truncation and/or a C-terminal truncation of a reference polypeptide or a 5′-terminal and/or 3′-terminal truncation of a reference polynucleotide).

A “fragment” is a portion of an amino acid sequence or a polynucleotide which is identical in sequence to but shorter in length than a reference sequence (e.g. relative to the amino acid sequence of any of SEQ ID NOs:1-6). A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least about 5, 10, 15, 20, 25, 30, 40, 50, 60, 70, 80, 90, 100, 150, 250, or 500 contiguous nucleotides or contiguous amino acid residues of a reference polynucleotide or reference polypeptide, respectively. In some embodiments, a fragment may have a length within a range bounded by any value selected from 5, 10, 15, 20, 25, 30, 40, 50, 60, 70, 80, 90, 100, 150, 250, or 500, contiguous nucleotides or contiguous amino acid residues of a reference polynucleotide or reference polypeptide, respectively. Fragments may be preferentially selected from certain regions of a molecule. The term “at least a fragment” encompasses the full length polynucleotide or full length polypeptide.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences. Homology, sequence similarity, and percentage sequence identity may be determined using methods in the art and described herein.

The phrases “percent identity” and “% identity,” for example, as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides. A “variant” may have substantially the same functional activity as a reference polypeptide. For example, a variant of HCHL1 may add ubitquitin to a substrate and/or may remove ubiquitin from a substrate.

Polynucleotides and Synthesis Methods

Polynucleotides and uses thereof may be disclosed herein such as polynucleotides encoding at least a fragment of the amino acid sequence of HCHL1 (e.g., a polynucleotide encoding any of SEQ ID NOs:1-6) or a variant thereof. Polynucleotides disclosed herein may include the nucleotide sequence of pAAV-hUCHL1p-HCHL1-IRES-mCherry (SEQ ID NO:7) and pAAV-CBA-UCHL1-IRES-mCherry (SEQ ID NO:8). The terms “polynucleotide (or “nucleic acid” and “oligonucleotide”) refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

A “fragment” of a polynucleotide is a portion of a polynucleotide sequence which is identical in sequence to but shorter in length than a reference sequence. As used herein, a reference sequence may include a polynucleotide sequence encoding UHCL1 (e.g., a polynucleotide sequence encoding any of SEQ ID NOs:1-6, a polynucleotide of SEQ ID NOs:7 or 8) or a variant thereof. A fragment may comprise up to the entire length of the reference sequence, minus at least one nucleotide. For example, a fragment may comprise from 5 to 1000 contiguous nucleotides of a reference polynucleotide. In some embodiments, a fragment may comprise at least 5, 10, 15, 20, 25, 30, 40, 50, 60, 70, 80, 90, 100, 150, 250, or 500 contiguous nucleotides of a reference polynucleotide; in other embodiments a fragment may comprise no more than 5, 10, 15, 20, 25, 30, 40, 50, 60, 70, 80, 90, 100, 150, 250, or 500 contiguous nucleotides of a reference polynucleotide; in further embodiments a fragment may comprise a range of contiguous nucleotides of a reference polynucleotide bounded by any of the foregoing values (e.g. a fragment comprising 20-50 contiguous nucleotides of a reference polynucleotide). Fragments may be preferentially selected from certain regions of a molecule. The term “at least a fragment” encompasses the full length polynucleotide. A “variant,” “mutant,” or “derivative” of a reference polynucleotide sequence may include a fragment of the reference polynucleotide sequence.

Regarding polynucleotide sequences and variants thereof, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The term “promoter” as used herein refers to a cis-acting DNA sequence that directs RNA polymerase and other trans-acting transcription factors to initiate RNA transcription from the DNA template that includes the cis-acting DNA sequence. Promoters may include eukaryotic promoters which function in eukaryotic cells and prokaryotic promoters which function in prokaryotic cells.

Eukaryotic promoters are known in the art and may include, but are not limited to, a promoter selected from the group consisting of CMV promoter (i.e., strong mammalian expression promoter from the human cytomegalovirus), CBA promoter (i.e., chicken β-actin promoter), UCHL1 promoter (i.e., promoter for ubiquitin carboxy-terminal hydrolase ligase 1 gene), EF1α promoter (i.e., strong mammalian expression from human elongation factor 1α), SV40 promoter (i.e., mammalian expression promoter from the simian vacuolating virus 40), PGK1 promoter (i.e., mammalian promoter from phosphoglycerate kinase gene), Ubc promoter (i.e., mammalian promoter from the human ubiquitin C gene), and human beta actin promoter (i.e., mammalian promoter from beta actin gene).

As used herein, the term “complementary” in reference to a first polynucleotide sequence and a second polynucleotide sequence means that the first polynucleotide sequence will base-pair exactly with the second polynucleotide sequence throughout a stretch of nucleotides without mismatch. The term “cognate” may in reference to a first polynucleotide sequence and a second polynucleotide sequence means that the first polynucleotide sequence will base-pair with the second polynucleotide sequence throughout a stretch of nucleotides but may include one or more mismatches within the stretch of nucleotides. As used herein, the term “complementary” may refer to the ability of a first polynucleotide to hybridize with a second polynucleotide due to base-pair interactions between the nucleotide pairs of the first polynucleotide and the second polynucleotide (e.g., A:T, A:U, C:G, G:C, G:U, T:A, U:A, and U:G).

As used herein, the term “complementarity” may refer to a sequence region on an anti-sense strand that is substantially complementary to a target sequence but not fully complementary to a target sequence. Where the anti-sense strand is not fully complementary to the target sequence, mismatches may be optionally present in the terminal regions of the anti-sense strand or elsewhere in the anti-sense strand. If mismatches are present, optionally the mismatches may be present in terminal region or regions of the anti-sense strand (e.g., within 6, 5, 4, 3, or 2 nucleotides of the 5′ and/or 3′ terminus of the anti-sense strand).

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions.” Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The terms “target,” “target sequence”, “target region”, and “target nucleic acid,” as used herein, are synonymous and refer to a region or sequence of a nucleic acid which is to be amplified, sequenced, or detected.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (TRES) or a 3′-UTR element, such as a poly(A)ₙ sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

Gene Therapy and Vectors for Expressing UCHL1 and Other Therapeutic Gene Products in the Motor Neurons of a Subject in Need Thereof

In some embodiments of the disclosed methods, a subject in need thereof may be administered gene therapy in order to express a heterologous DNA, such as DNA encoding UCHL1 or a variant thereof, or other therapeutic gene product (e.g., therapeutic RNA or protein) in order to treat a neurological disease, disorder, or injury, such as a neurological disease, disorder, or injury associated with the activity of upper motor neurons. In some embodiments of the disclosed methods, a subject in need thereof is administered a vector, which may be a viral vector or non-viral vector in order to express a heterologous DNA, such as DNA encoding UCHL1 or a variant thereof, in order to treat a neurological disease, disorder, or injury, such as a neurological disease, disorder, or injury associated with the activity of upper motor neurons. Gene therapy and vectors, including viral and non-viral vectors for gene transfer or expression, RNAi, and CRISPR/Cas9 genome editing for therapeutic purposes are known in the art. (See, e.g., Keeler et al., “Gene Therapy 2017: Progress and Future Directions,” Clin Transl Sci. 2017 July; 10(4):242-248; Dunbar et al., “Gene therapy comes of age,” Science, 12 Jan. 2018: Vol. 359, Issue 6372; Kumar et al., “Clinical development of gene therapy: results and lessons from recent successes,” Molecular Therapy—Methods & Clinical Development (2016) 3, 16034; the contents of which are incorporated herein by reference in their entireties).

In some embodiments of the disclosed methods, a subject in need therefore is administered a vector in order to express a heterologous DNA, such as DNA encoding UCHL1 or a variant thereof, in order to treat a neurological disease, disorder, or injury, such as a neurological disease, disorder, or injury associated with the activity of upper motor neurons. In the disclosed methods, any suitable vectors may be used for expressing UCHL1 or a variant thereof in a subject in need thereof. The term “vector” refers to some means by which heterologous DNA, such as DNA encoding UCHL1 or a variant thereof, can be introduced and expressed in a cell or cells forming a tissue. There are various types of vectors suitable for the disclosed methods, and suitable vectors may include viral vectors. As used herein, a “viral vector” (e.g., a viral vector derived from an adenovirus or adeno-associated virus, Sendai virus, or measles virus vector) refers to recombinant viral nucleic acid that has been engineered to express a heterologous polypeptide (e.g., UCHL1 polypeptide or a variant thereof). The recombinant viral nucleic acid typically includes cis-acting elements for expression of the encoded heterologous polypeptide (e.g., a polypeptide comprising the amino acid sequence of any of SEQ ID NOs:1-6). Recombinant viral vectors may comprise all or a fragment of the polynucleotide sequence of pAAV-hUCHL1P-UCHL1-IRES-mCherry (SEQ ID NO:7) or pAAV=CBA-HCHL1-IRES-mCherry (SEQ ID NO:8). In some embodiments, recombinant viral vectors may comprise one or more polynucleotide sequences encoding one or more therapeutic polypeptides.

The recombinant viral nucleic acid typically is capable of being packaged into a helper virus that is capable of infecting a host cell. For example, the recombinant viral nucleic acid may include cis-acting elements for packaging. Typically, the viral vector is not replication competent or is attenuated. An “attenuated recombinant virus” refers to a virus that has been genetically altered by modern molecular biological methods (e.g., restriction endonuclease and ligase treatment, and rendered less virulent than wild type), typically by deletion of specific genes. For example, the recombinant viral nucleic acid may lack a gene essential for the efficient production or essential for the production of infectious virus. The recombinant viral nucleic acid, packaged in a virus (e.g., a helper virus) may be introduced into a human subject by standard methods.

Numerous virus species can be used as the recombinant virus vectors for the pharmaceutical composition disclosed herein. A preferred recombinant virus is adeno-associated virus (AAV). In some embodiments, suitable AAV vectors for the disclosed compositions and methods may include AAV serotypes 1, 2, 3, 4, 5, 6, 7, 8, or 9. Adeno-associated viral (AAV) vectors are recognized as an emerging gene therapy platform for the treatment of neurological diseases. (See, e.g., Deverman et al., “Gene therapy for neurological disorders: progress and prospects,” Nature Reviews Drug Discovery,” 17 641-659 (2018), the content of which is incorporated herein by reference in its entirety). Others include adenoviruses, retroviruses that are packaged in cells with amphotropic host range, vaccinia virus, canarypox, Sendai virus, measles virus, Yellow Fever vaccine virus (e.g., 17-D or similar), attenuated or defective DNA viruses, such as but not limited to herpes simplex virus (HSV), papillomavirus, and Epstein Barr virus (EBV).

In certain exemplary embodiments, the recombinant expression vectors disclosed herein comprise a nucleic acid sequence in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more polypeptides and/or proteins described herein is linked to the regulatory sequence(s) in a manner which allows for expression of the nucleotide sequence (e.g., in an in vitro transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Suitable promoters for use in the disclosed expression vectors may include without limitation, CMV promoter (i.e., strong mammalian expression promoter from the human cytomegalovirus), CBA promoter (i.e., chicken β-actin promoter), UCHL1 promoter (i.e., promoter for ubiquitin carboxy-terminal hydrolase ligase 1 gene), EF1α promoter (i.e., strong mammalian expression from human elongation factor 1α), SV40 promoter (i.e., mammalian expression promoter from the simian vacuolating virus 40), PGK1 promoter (i.e., mammalian promoter from phosphoglycerate kinase gene), Ubc promoter (i.e., mammalian promoter from the human ubiquitin C gene), and human beta actin promoter (i.e., mammalian promoter from beta actin gene). In some embodiments, the viral vector comprises the UCHL1 promoter, driving the expression of one or more therapeutic gene products, such as a therapeutic RNA and/or protein.

By way of example, but not by way of limitation, in some embodiments, the vector comprises the human UCHL1 promoter, or a variant thereof, or the mouse UCHL1 promoter, or a variant thereof. The human UCHL1 promoter sequence (HRPM17682, NM004181) is provided below (SEQ ID NO: 16):

TGGAGCCCAGTTTAGCAGGGTTTACTTCTCAGTTTTACTTTTCAATTGTG
ACCGTAAGCAAATTAACTTCTCTAGGCCTGGGATTCTGATCTGTAAAATT
GCACTAATATGAGTCTCTTCACGGCTCCTCTAAGGATTAAATGAGAGACA
CATGCAAAGGATCCCCAAAAACAATAACTCAAAAAATGTTGATTCCCTCC
CTTCCCTCTGTCATCTGTTAACCTCAACTTCCTAAATAGAAGGTCTATTC
TTTTACCATCATCATTATTCTCTTCGGTGCCTATTTTTAAAAAATACTCA
ACCTTCTTGCTTCCTTCGCTACCTAAGTATTTCTGCAAGCCCACTTTGTT
CTGCAGCTTAGCTTTCCTGGCACAATTCTTATAGATTTTGGTCCCTTTTA
AAATTCATTCTTCAGCAAATGCTTTCTCTCTCCATCTTTTGACTAGAGAT
CATTAGAGATCACCTGAGATCATTAGAGAACAGTGGTTTCCTTGGTTGCC
ATTCCCTTTCTTCTTCATTGGGAGTATTCTGCGGTGAACTCAGACATTTT
ATTTTTCAAAGCTTCCCATTCTTTTAAAAATGCTTTTCCTTTTACAGCCT
CTCGCTCAAAATCATACCCATCTTTTCCCTGGATCTGTTTTCTCAAGTCT
CCAATCGCCTGCCTTCTTTGTGTCTTGTATTACCCTCACATCCCCCAGCT
TTCTACTGCTCTCCCAGGACCAACCATTTCTTCCGCGGGAGTCACATTAC
ATCAGCATTCCTAATGCAGTATCTGTTATCTACCAGATTCTGTTTTATTC
TAGGTAGTCACTTAAAAACGAACCTCGGTACTGGTCTGACTTAACATGGA
GGAGGAATTGTCTAAGGTTAAACGCAAACTGCTGAGAGATTTGGGGCGGG
GGGCACACATTTACATTCATTCGTATTAAATATATACCTGTTGAATTTGT
GCTTTTTCTCAAATGCTTCAGAGACTCGAGCTTTAGAGTAATTGGGATGG
TGAAAGGATGGGTTTCCAGAAACTTCGCCCAAAATTAAAGACTCCATCAA
AAGGACTGCTCCATACACTCAAGGAACACCCACCAACAAATCCCGTCTCC
ACAACCACCAGATTATCTCACCGGCGAGTGAGACTGCAAGGTTTGGGGGC
CCGGCCGTACCACTCCGCGCTGCGCACGGGGGGTTCGTACCCATCTGGCC
GCGACCGTCCGTTTCCCCCTCGCTTGGTTCTGCCCCTGCTCCCCCTGCAC
AGGCCTCACAGTGCGTCTGGCCGGCGCTTTATAGCTGCAGCCTGGGCG

The mouse UCHL1 promoter (MPRM41981, NM_011670 is provided below (SEQ ID NO: 17):

CCCAAGCTCAGTGACCAGTGGGAGTAAATGCTTGCTTGTGCAGTATTTGT
GATGAATGGATAAATGAAGAAAACTGGAAATGTAGTATGTCTCTGCCAAA
AGACAGACATGCACACAGTCTACCCAGGTGTACTGGGTTATGTGTCCGTC
TGTCTTGTATTTTTTTTTTTTATTAACTGCATTGAGATGTAATTTACATA
GTATAAAATTCACTCAGAATCTTACTAATGCCTTCTTGCCTCTCACACTT
GGTAAGTGAAGGTCATTTTTTTTTCCCTGATGGTGAAACTGGGAAGAAAA
TTGAAGTAGCACATTGCTGAGTCTAAAACAGAAAATGGACCCTTCCAGCC
TCATTTACCAGGTTTTGTCTTCCTGCCTCATTTTCCAGCTGTAGCACCAA
GCAAATGAACATCTCTGAACCCTGGATCCTGATGTATAAAATTTCACAAA
TACAATATTGGACCCTCTCAGGGTGAAATAAGATGGCCTGGTAAAGGATC
CAGCTGGAGAGGCAGTGCTGTTAAAACAAGGTTGGTTCCCTCCATCCTAT
GCCTTGCTTACCTCGGGCTAGCAAAAAGCATGTCTGTTTTTCACCATTTG
TTCTGAGCATATTTTTTAAAATACCCAACCTGCCTGCATCCTTAGCTACC
TCATTCTGTGATTTAGCATCCCTGACAATTGATACAGATCTGGCCCCTCC
TTCTACTCCGCCTTCAGCACAAGCGTTTGCCTTCCATCCTTTGAAATGTG
CCTGTTAGAAAGAACACCCCCGCCCCCATCCCTGGAGAACCGCGGGCTCT
CCTCCGTTGCTATTCCCTTTCTTCTGCGCCCAAAGTATTGTGGACCAAAA
TCTTATTATCCCAAACCCCTGTTCTTTGAAAAAAGGTTTCCCCTATTTTT
TTTTTAAATGCTTTTTCCTTTAAGCCTCTAGTCTCAAATCATCTATCTTT
TCTTTGGTCTGTTTCTAAAGTCCTCCGGCGCCCGTATTCTATGTGTCTCG
CATGTACTGCTTCCCAGCGACCCACCGGTTCTTCCCCTGGAGTCACATTA
CATCAGCTTCACTAATGCACTCCGTATCAGATGTTTTATTCTAGGCAGTC
CATTAAAAACCGAGCCTGGACACAGGTTGAATTTAACATGGAGGACAGAT
TGTCTAAGGTTAAGTACAAACTGTTCCGAGATGGGCGGGGGGGGGGAGGG
AGGAAGACCGACAAATATTTATATTCATATTAAACACTACATATACCTGT
TGATTTTGTGCTTTTTTTTCTCTCTCTCTCAATGTTTCAGAGACCCTCGA
GCTTTGGAGTGATTGAGACGACAGAGGAGGGTTTCCAGAAATTTCGCTCC
AAATTGAAAGCTCCCTCAAATAGGCTGCTTCATGCGCTCAAGGAACACCC
ACCAACAAAACCCATCTCCACAACCACCAGATTAGCTCACCGGCGAGTGA
GACTGCAAGGTTTGGGGGCTTGGCTTGTACCATTCTGTGCGGTGCATGGG
GGAGTTCGAGCCCTCTTGGCCTTCCTCCTCTGCTTGTTTCTGCTCCCGTC
TCCCCTGCTCAGGCTTTCCCAGTGAGCGAGGCCGGCGCTTTATAACAGCA
GCCTGGGCGGCTCCACCGGCTGTTTTTTCGGCTCCTCGGGTTTGTGTCTG
CAGGTGCCATCCGCGA

In some embodiments, therapeutic genes (encoding a therapeutic gene product such as a protein or nucleic acid) comprise one or more of UCHL1, or other therapeutic gene to, for example replace an absent gene product or to correct a defective gene product. By way of example, therapeutic genes or gene targets are those implicated in a neurological disease or condition. By way of example, but not by way of limitation, exemplary therapeutic genes or gene targets may include, but are not limited to those genes and their gene products implicated in amyotrophic lateral sclerosis (ALS) (see e.g., Dervishi et al., “Protein-protein interactions reveal key canonical pathways, upstream regulators, interactome domains, and novel targets in ALS,” Scientific Reports (2018) 8:14732 |DOI.10.1038/s41598-018-32902-4, incorporated herein by reference in its entirety, at Table 1, which is reproduced below).

Causative Genes*
ALS2, ALS3, ALS7, ANG, ANXA11, ATXN2, CFAP410, C9orf72, CHCHD10, CHMP2B, DAO, DCTN1, ELP3,
ERBB4, Erlin1, FIG4, FUS, HNRNPA1, LMNB1, MATR3, NEFH, NEK1, OPTN, PFN1, PRPH, SETX, SIGMAR1,
SOD1, SPAST, SPG11, SQSTM1, TAF15, TARDBP, TIA1, TUBA4A, UBQLN2, UNC13A, VAPB, VCP.
Associated and Disease Modifier Genes*:
AGT, ALAD, APEX1, APOE, AR, ARHGEF28, ATRN, ATXN1, B4GALT6, BCL6, BCL11B, BIRC6, C1orf27,
C1QTNF7, CCNF, CCS, CDH13, CDH22, CHGB, CNTF, CNTN4, CNTN6, CREB3L2, CRIM1, CRYM, CSNK1G3,
CST3, CX3CR1, CYP2D6, DCC, DIAPH3, DISC1, DOC2B, DPP6, DYNC1H1, EFEMP1, EPHA4, EWSR1, FEZF2,
FGGY, GARS, GLE1, GRB14, GRN, HEXA, HFE, HNRNPA2B1, ITPR2, KCNN1, KDR, KIF5A, KIFAP3, LIF, LIPC,
LOX, LUM, MAOB, MAPT, MOBP, MT-ND2, NAIP, NETO1, NIPA1, NT5C1A, OGG1, OMA1, PARK7, PCP4,
PEAK1, PLEKHG5, POLDIP2, PON1, PON2, PON3, PSEN1, PVR, RAMP3, RBMS1, RNASE2, RNF19A, SARM1,
SCFD1, SCN7A, SELL, SEMA6A, SLC1A2, SLC39A11, SMN1, SMN2, SNCG, SOD2, SOX5, SPG7, SS18L1, STX12,
SUSD1, SYNE, SYT9, TBK1, TRPM7, VDR, VEGFA, VPS54, WDR49, ZFP64, ZNF746, ZNF512B, ZSCAN5B.

In some embodiments, a therapeutic gene may include genes involved in hereditary spastic paraplegia (HSP) or primary lateral sclerosis (PLS), both of which are motor neuron diseases, affecting mostly the upper motor neurons (UMMs) in patients. Exemplary therapeutic genes, or therapeutic gene targets include but are not limited to those describe in Gozutok, et al., (Mutations and Protein Interaction Landscape Reveal Key Cellular Events Perturbed in Upper Motor Neurons wit HSP and PLS”, Brain Sci. 2021, 11, 578, incorporated herein by reference in its entirety, see e.g., Table 1 and 2, shown below).

HSP CAUSATIVE GENES
ALS2, ALDH18A1, AP4B1, AP4E1, AP4M1, AP4S1, AP5Z1, ARL6IP1, ATAD3A, ATL1, B4GALNT1, BICD2,
BSCL2, C19orf12, CAPN1, CCT5, CYP2U1, CYP7B1, DDHD1, DDHD2, ERLIN2, ENTPD1, EPT1, ERLIN1,
FARS2, FA2H, GBA2, IBA57, KCNA2, KIAA0196/WASHC5, KIF1A, KIF5A, KY, L1CAM, LYST, NIPA1,
NT5C2, PCYT2, PLP1, PNPLA6, RAB3GAP2, REEP1, REEP2, RTN2, SETX, SLC33A1, SPAST, SPG11,
SPG21/ACP33, SPG7, SPG80/UBAP1, TECPR2, TFG, TUBB4A, VCP, VPS37A, ZFYVE26, ZRYVE27
HSP ASSOCIATED GENES
ABCD1, ADAR, ALDH3A2, AMPD2, ARSA, ARSI, ATP13A2, CPT1C, ELOVL1, FLRT1, GALC, GCH1, GRN,
HSPD1, HSP60, IFIH1, KIF1C, MAG, MARS, MFN2, MT-ATP6, OPA1, OPA3, PGAP1, PLA2G6, POLR3A,
SERAC1, SLC16A2, SPART, SPG20, TRPV4, USP8, WDR48, ZFR
HSP LINKED GENES
C12orf65, CYP27A1, GAD1, GJC2, KLC2, RIPK5/DSTYK, UCHL1
PLS GENES
CAUSATIVE: ALS2 ASSOCIATED: ALS15, C9ort72, DCTN1, ERLIN2, PARK2 LINKED: FIG4, SPG7

| Protein | Binding Partners |
|---|---|
| ALDH18A1 | ADRB2, AGTRAP, C1QBP, CDK2, CMTM5, CUL3, EED, G3BP1, GABRA2, GOLT1B, HDAC5, MOV10, |
| | MRPL58, MYC, NFATC2, NR3C1, NTRK1, NXF1, SHMT2, SIRT7, STAU1, TCF3, VCP |
| AP4E1 | ALB, AP4B1, AP4M1, AP4S1, ARF1, ELAVL1, GOLIM4, LAMA1, MAP3K4, SUV39H2, TEPSIN, TFAP2A, |
| | TMEM17, XPO1, YME1L1 |
| AP4S1 | AP4B1, AP4E1, AP4M1, APP, CDC73, GOLIM4, GRB2, HLTF, LAMA1, MAP3K4, SUV39H2, TEPSIN, |
| | TFAP2A, YME1L1 |
| ERLIN1 | AMFR, C1QBP, CD2AP, CFTR, CHMP4B, COX15, CUL7, DUSP3, EDEM3, FAF2, FANCD2, FBXO6, GABRA2, |
| | GOLT1B, HNF4A, INSIG1, ITPR1, Ktn1, NTRK1, PKN2, RAB5C, RAB7A, RNF170, SPAST, STOM, SUZ12, |
| | SYVN1, TMED2, TRAF6, TRIM25, TSG101, UBC, VAPA, VDAC1 |
| FARS2 | AGTRAP, APPL1, CMTM5, CUL, HNRNPA1, ISG15, KRT31, KRT40, KRTAP10-3, KRTAP10-7, KRTAP10-9, |
| | LOC100996763/NOTCH2NL, MID2, MRPL58, NXF1, PDHA1, SHMT2, STAT5A, TRIM27, TRIM54 |
| GRN | AHCYL2, ATN1, BRCA1, CCDC8, CDK2, CDK9, CFTR, CSNK2B, CTTN, CUL7, EED, EGFR, FBXO6, GRIA2, |
| | HECW2, HSP90AA1, HSP90AB1, HSPA4, KRTAP10-7, MAPK1, NF2, NPM1, NTRK1, NXF1, PIK3R2, POT1, |
| | PPP2CA, PRKAA1, RAC1, SIRT3, TNFRSF1A, TUBA1C, VHL |
| KIF1A | APP, AR, COX15, DLG4, EGFR, FMR1, FXR2, KIF1BP, LOC100996763/NOTCH2NL, MDFI, MID2, MTUS2, |
| | NTRK1, PLSCR1, PPP2CA, PSMA3, RAC1, RBPMS, SIRT1, SIRT7, SP1, TRAF1, TRIM27, UBC |
| KIF1C | ARF1, BICD2, HSPA8, KIF1BP, KSR1, MYH9, PRKAA1, STAU1, TRIM25, USP21, YWHAB, YWHAE, |
| | YWHAG, YWHAQ, YWHAZ |
| KIF5A | ACTB, APP, BICD2, DCTN1, DLG4, FANCD2, FMR1, GRB2, GRIA2, HACD3, KIF5B, KIF5C, KLC2, MDM2, |
| | MYCL, STAU1, TSG101, YAP1, YWHAE, ZFYVE27 |
| KLC2 | AIMP2, APP, CDH1, EZH2, GRIA2, KCNMA1, KIF5B, KIF5C, MYCL, NTRK1, PIK3R3, PPP4C, SCAMP2, |
| | SOD1, VCP, WDR70, YWHAB, YWHAB, YWHAG, YWHAH, YWHAQ, YWHAZ |
| MARS | AIMP2, BRCA1, CCDC8, CDK9, COPS5, CRY2, CUL1, CUL3, CUL7, CYLD, DLST, EED, EGFR, ESR1, |
| | FANCD2, FBXO25, FBXO6, FN1, G3BP1, HACD3, HDAC5, HSP90AA1, HUWE1, IKBKG, ILK, ITGA4, Ktn1, |
| | MAP3K1, MAP3K3, MCC, MCM2, MDM2, MYC, NTRK1, PDHA1, PKN2, RARS, RNF2, TRAF6, VCAM1 |
| PARK2 | ABL1, BAX, CCND1, CCT2, CDK5, CTNNB1, CUL1, EGFR, HDAC6, HSPA9, HSPD1, IKBKG, PDHA1, |
| | RNF31, SNCA, STUB1, TARDBP, TCP1, TP53, TRAF2, TUBB, VDAC1 |

Therapeutic nucleic acids (e.g., RNA, such as siRNA) may also be driven by the UCHL1 promoter.

In some embodiments, the UCHL1 vector comprises SEQ ID NO: 16, or a variant thereof, wherein the variant is at least about 80%, 85%, 90%, 95%, 96%, 97%, 98%, or 99% identical to SEQ ID NO: 16.

Pharmaceutical Formulation

The disclosed therapeutic agents may be formulated as pharmaceutical compositions for administering to a subject in need thereof. The disclosed pharmaceutical compositions may include: (a) a therapeutic agent as discussed herein (e.g., an effective amount of a therapeutic agent for treating a disease or disorder associated with corneal vascularization); and (b) one or more pharmaceutically acceptable carriers, excipients, or diluents.

The therapeutic agents utilized in the methods disclosed herein may be formulated as a pharmaceutical composition that includes one or more carrier agents, binding agents, filling agents, lubricating agents, suspending agents, buffers, wetting agents, and/or preservatives. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

The therapeutic agents utilized in the methods disclosed herein may be formulated as a pharmaceutical composition for delivery via any suitable route. For example, the pharmaceutical composition may be administered topically to the surface of a cornea.

The therapeutic agents utilized in the methods disclosed herein may be administered in conventional dosage forms prepared by combining the active ingredient with standard pharmaceutical carriers or diluents according to conventional procedures well known in the art. These procedures may involve mixing or dissolving the ingredients as appropriate to the desired preparation.

Pharmaceutical compositions comprising the therapeutic agents may be adapted for administration by any appropriate route, for example topically to the surface of the cornea. Such formulations may be prepared by any method known in the art of pharmacy, for example by bringing into association the active ingredient with the carrier(s) or excipient(s).

Exemplary Advantages and Applications

Advantages of the disclosed technology may include but are not limited to: (i) Currently, there is no existing technology to selectively target the upper motor neurons. Even though there is virus mediated gene delivery none of them are specific to upper motor neurons; (ii) Targeting specific neuron population that is clinically relevant offers great advantages; (iii) Higher levels of upper motor neuron transduction; (iv) Improvement of gene therapy approaches to upper motor neurons without affecting other cells or neurons in the CNS. The specificity of transduction offers great advantages; (v) We found the importance of UCHL1 protein in sustaining and improving the health of diseased upper motor neurons. Therefore, targeted delivery of UCHL1 to the diseased upper motor neurons offer a therapeutic strategy to improve the health and connectivity of diseased and injured upper motor neurons in patients.

Applications of the disclosed technology may include but are not limited to: (i) Directed gene delivery and cellular therapy for the upper motor neurons in the cerebral cortex. Since upper motor neurons die in hereditary spastic paraplegia, primary lateral sclerosis and amyotrophic lateral sclerosis patients, gene delivery to these clinically important neuron population has significant health implications, and because long-term paralysis develops in spinal cord injury patients because of upper motor neuron degeneration, directed gene delivery to upper motor neurons within the context of spinal cord injury is also clinically relevant; (ii) Modulation of upper motor neuron activity; (iii) Improvement of upper motor neuron health; (iv) Improvement of upper motor neuron connectivity; and (v) Gene delivery and modulation of gene expression in upper motor neurons and related neuron. In particular, methods and compositions disclosed here can be used to provide UCHL1 gene therapy directed specifically to upper motor neurons in the treatment of disease and injuries that affect upper motor neurons.

Importance of upper motor neurons: Upper motor neurons are the cortical component of the motor neuron circuitry, which initiates and modulates voluntary movement. For every movement that we make, our cortex is responsible for the order of movement that goes from brain to the spinal cord. Even though the spinal motor neurons have direct connection with the muscle, the initial order to move the muscle comes from the brain. Therefore, when the brain spinal cord connection is lost, patients cannot perform voluntary movement.

Upper motor neurons are very few in numbers. Even in the motor column of the motor cortex, their presence is less than one percent of the total neurons and cells. They are located in layer V of the motor cortex with other excitatory neurons, such as callosal projection neurons, and they receive a wide variety of input from a broad spectrum of neurons, such as long distance projection neurons and local circuitry neurons. They extent along apical dendrites to the top layers of the cortex, and the apical dendrite is very important for their proper modulation by other cortical neurons. They receive most of their input from layer 2-3 of the motor cortex and they have a unique ability to collect, integrate, translate the input they receive from different regions of the brain and different neurons of the cerebral cortex and to turn this information into a signal that is propagated to distinct targets within the spinal cord. They are the only neurons in humans that have the ability to do this, therefore, their degeneration leads to paralysis in humans and their degeneration is detrimental in patients.

There are many diseases, which are characterized by the degeneration of upper motor neurons, such as hereditary spastic paraplegia (HSP), primary lateral sclerosis (PLS), and amyotrophic lateral sclerosis (ALS). In ALS the spinal motor neurons also degenerate, but the upper motor neuron loss is the characteristic of ALS; without upper motor neuron degeneration patients are not diagnosed with ALS. In addition, the upper motor neuron degeneration is one of the causes of long-term paralysis in spinal cord injury patients. During injury, the corticospinal tract is severed, and the connection from the brain to the spinal cord is hampered, followed by axonal degeneration and cell death. When the upper motor neurons die, the paralysis becomes stable in patients. Therefore, it is important to improve the health of upper motor neurons both within the context of neurodegeneration and injury.

UCHL1: Ubiquitin carboxyl hydrolase ligase 1 (UCHL1) is a special deubiquiating enzyme, which has the ability to add and remove an ubiquitin. By doing so, it not only determines which proteins to be recycled, but it also has an impact on the translocation of proteins to different compartments and their transport within the cell. UCHL1 also makes sure that there is free ubiquitin in the cell, which is very important for protein degradation and turnover, and UPS system to work properly. UCHL1 is mainly expressed in neurons. It is highly present in the cortex and the upper motor neurons express very high levels of UCHL1 throughout life.

Current therapies: Currently there are no effective therapies for any of the motor neuron diseases and spinal cord injury. There is no gene delivery therapy for the upper motor neurons.

Generated knowledge prior to this application:
1) We found that UCHL1 (ubiquitin carboxyl hydrolase ligase 1) is important for the health and stability of upper motor neurons. The upper motor neurons are called corticospinal motor neuron (CSMN) in mice and Betz cells in patients, and they express high levels of UCHL1.
2) The UCHL1 expression in CSMN is so profound that when a reporter line which express eGFP under the control of UCHL1 promoter is generated, only the CSMN become genetically labeled with eGFP expression in the motor cortex (Yasvoina et al).
3) When transgenic mice are generated in which there is no UCHL1 function, the mice develop neurodegeneration, with profound upper motor neuron loss and motor function defects. Even though the spinal motor neurons are relatively intact, the upper motor neurons begin to degenerate very early in the motor cortex. Even though in these mice UCHL1 is ablated from the whole body, the most profound impact is on the upper motor neurons, further suggesting a special importance of UCHL1 for the upper motor neurons.
4) When transgenic mice are generated in which UCHL1 function is depleted only from layer V of the motor cortex, or from the spinal cord, the upper motor neurons display degeneration regardless of the UCHL1 levels in the spinal cord, further revealing that the upper motor neurons require the function of UCHL1, and their degeneration is not secondary to spinal motor neuron loss.
5) When the UCHL1 gene is given back and expressed in the motor cortex of the UCHL1 null mice with a gene replacement strategy, the upper motor neurons regain structural integrity and do not degenerate. Therefore, giving back UCHL1 is sufficient to reverse the degeneration phenotype in the UCHL1 null mice.
6) More interestingly, when the UCHL1 gene is introduced to the hSOD1G93A mice, a well-respected ALS mouse model, which displays upper motor neuron loss, the diseased upper motor neurons regain cytoarchitectural integrity, stability and health. This is very important, as UCHL1 treatment can have a broad impact for improving the health of diseased upper motor neurons in general.

UCHL1 gene delivery approaches: Since we realized the importance of UCHL1 as a potential treatment strategy for upper motor neurons, and because upper motor neurons are very few in numbers we developed targeted therapies that impact only the upper motor neurons, without affecting other cells or neurons in the cortex. Previous approaches included non-specifically transducing many different cell types and neurons, but we think for building effective and long-term treatment strategies, we need to deliver solutions directly to the neuron in need without perturbing the balance or the homeostatis in other cells/neurons/circuitries/systems. To this end:
1) We tested different AAV serotypes (i.e. AAV 2, 3, 4, 5, 6, 7, 8, 9) and their ability to transduce cortical neuron in the motor cortex, and found that AAV2 is more effective in transducing cortical neurons in the motor cortex. So we first identified the best serotype to use. (Jara, Nature Gene Therapy).
2) We tested different promoters, such as CMV, CBA, and most recently human UCHL1. We published that CMV and CBA promoters are effective for selective transduction of CSMN, and as disclosed herein, we found that the UCHL1 promoter is less effective but more specific to CSMN (see Examples, below). Thus, the UCHL1 promoter can be used with any expression vector for targeted gene therapy, e.g., as a means to direct therapeutic RNAs and/or proteins specifically to CSMN. Given that both GFP and mCherry, two proteins that are not naturally occurring in either mouse or Macaque, are specifically and effectively expressed in CSMN in both species provides strong support for the promoter's ability to direct expression of a wide range of expression products (e.g., therapeutic polypeptides in addition to UCHL1 or therapeutic nucleic acids) in the CSMN.
3) We generated expression vectors to introduce UCHL1 gene expression in the upper motor neurons.
4) These expression vectors are coated with AAV2-2 for in vivo gene delivery experiments, and are injected into the motor cortex of UCHL1 null mice and hSOD1^{G93A} mice. Since the expression vectors contain mCherry gene the neurons that are transduced can be visualized with red fluorescence and can be traced for detailed cellular analyses. Neurons that are transduced are alive, do not show signs of cellular toxicity and most importantly they improve overall health in disease models.
5) We also tried experiments with Macaque monkeys to investigate whether human UCHL1 promoter driven mCherry expression is mostly seen in the Betz cells of the Macaque monkey brain. Our preliminary results are very encouraging. Transduction was very specific, other cortical neurons and cells were not transduced. We have seen the apical dendrites of the Betz cells that are selectively transduced and to visualize the soma of the Betz cells we are now using clearance technology.

Therefore, because UCHL1 protein is important for the health and function of upper motor neurons, and because in the absence of UCHL1 it is mainly the upper motor neurons that die, and, as disclosed herein, because the introduction of UCHL1 to a UCHL1 null mice and more importantly to the SOD1 disease model improves the health of this clinically important neuron population, we propose that targeted gene delivery approaches that affect only the upper motor neurons with UCHL1 will promote their neuronal integrity and overall health. Because upper motor neuron health and function is of utmost importance for the initiation and modulation of movement, improvement of upper motor neuron health will have a direct impact on the motor neuron circuitry that degenerates in patients.

### Illustrative Embodiments

The following embodiments are illustrative and should not be interpreted to limit the scope of the claimed subject matter.

Embodiment 1. A method for treating a neurological disease, disorder, or injury associated with upper motor neuron activity in a subject in need thereof, the method comprising administering to the subject a therapeutic agent that results in an increase in the concentration of ubiquitin carboxy-terminal hydrolase ligase 1 (UCHL1) in upper motor neurons of the subject relative to the concentration of HCHL1 in the upper motor neurons of the subject prior to administering the therapeutic agent.

Embodiment 2. The method of embodiment 1, wherein the subject has amyotrophic lateral sclerosis (ALS).

Embodiment 3. The method of embodiment 1, wherein the subject has hereditary spastic paraplegia (HSP).

Embodiment 4. The method of embodiment 1, wherein the subject has primary lateral sclerosis (PLS).

Embodiment 5. The method of embodiment 1, wherein the subject has a spinal cord injury.

Embodiment 6. The method of any of the foregoing embodiments, wherein the therapeutic agent is administered to the motor neurons of the subject.

Embodiment 7. The method of any of the foregoing embodiments, wherein the therapeutic agent is a vector that expresses UCHL1 or a variant thereof comprising an amino acid sequence having at least about 80% sequence identity to any of SEQ ID NOs:1-6.

Embodiment 8. The method of any of the foregoing embodiments, wherein the therapeutic agent is a viral vector that expresses UCHL1 or a variant thereof comprising an amino acid sequence having at least about 80% sequence identity to any of SEQ ID NOs:1-6.

Embodiment 9. The method of any of the foregoing embodiments, wherein the therapeutic agent is an adenovirus-associated viral (AAV) vector that expresses UCHL1 or a variant thereof comprising an amino acid sequence having at least about 80% sequence identity to any of SEQ ID NOs:1-6.

Embodiment 10. The method of any of the foregoing embodiments, wherein the therapeutic agent is an adenovirus-associated viral (AAV) vector serotype 1, 2, 3, 4, 5, 6, 7, 8, or 9 that expresses UCHL1 or a variant thereof comprising an amino acid sequence having at least about 80% sequence identity to any of SEQ ID NOs:1-6

Embodiment 11. The method of any of the foregoing embodiments, wherein the therapeutic agent is an AAV2 vector that expresses UCHL1 or a variant thereof comprising an amino acid sequence having at least about 80% sequence identity to any of SEQ ID NOs:1-6.

Embodiment 12. The method of any of the foregoing embodiments, wherein the therapeutic agent is a vector that expresses UCHL1 or a variant thereof comprising an amino acid sequence having at least about 80% sequence identity to any of SEQ ID NOs:1-6 utilizing a promoter selected from the group consisting of CMV promoter (i.e., strong mammalian expression promoter from the human cytomegalovirus), CBA promoter (i.e., chicken β-actin promoter), UCHL1 promoter (i.e., promoter for ubiquitin carboxy-terminal hydrolase ligase 1 gene), EF1α promoter (i.e., strong mammalian expression from human elongation factor 1 α), SV40 promoter (i.e., mammalian expression promoter from the simian vacuolating virus 40), PGK1 promoter (i.e., mammalian promoter from phosphoglycerate kinase gene), Ubc promoter (i.e., mammalian promoter from the human ubiquitin C gene), and human beta actin promoter (i.e., mammalian promoter from beta actin gene).

Embodiment 13. The method of any of the foregoing embodiments, wherein the therapeutic agent is a vector that expresses UCHL1 or a variant thereof comprising an amino acid sequence having at least about 80% sequence identity to any of SEQ ID NOs:1-6 utilizing the promoter for the human HCHL1 gene.

Embodiment 14. A vector that is capable of expressing UCHL1 in upper motor neurons of a subject in need thereof.

Embodiment 15. The vector of embodiment 14, wherein the vector is capable of expressing UCHL1 or a variant thereof comprising an amino acid sequence having at least about 80% sequence identity to any of SEQ ID NOs:1-6 in the upper motor neurons of the subject in need thereof.

Embodiment 16. The vector of embodiment 14 or 15, wherein the vector is a viral vector.

Embodiment 17. The vector of any of embodiments 14-16, wherein the vector is an adenovirus-associated viral (AAV) vector.

Embodiment 18. The vector of any of embodiments 14-17, wherein the vector is an adenovirus-associated viral (AAV) vector serotype 1, 2, 3, 4, 5, 6, 7, 8, or 9.

Embodiment 19. The vector of any of embodiments 14-18, wherein the vector is adenovirus-associated viral (AAV) vector serotype 2.

Embodiment 20. The vector of any of embodiments 14-19, wherein the vector expresses UCHL1 or a variant thereof comprising an amino acid sequence having at least about 80% sequence identity to any of SEQ ID NOs:1-6 utilizing a promoter selected from the group consisting of CMV promoter (i.e., strong mammalian expression promoter from the human cytomegalovirus), CBA promoter (i.e., chicken β-actin promoter), UCHL1 promoter (i.e., promoter for ubiquitin carboxy-terminal hydrolase ligase 1 gene), EF1α promoter (i.e., strong mammalian expression from human elongation factor 1 α), SV40 promoter (i.e., mammalian expression promoter from the simian vacuolating virus 40), PGK1 promoter (i.e., mammalian promoter from phosphoglycerate kinase gene), Ubc promoter (i.e., mammalian promoter from the human ubiquitin C gene), and human beta actin promoter (i.e., mammalian promoter from beta actin gene).

Embodiment 21. The vector of any of embodiments 14-20, wherein the vector expresses UCHL1 or a variant thereof comprising an amino acid sequence having at least about 80% sequence identity to any of SEQ ID NOs:1-6 utilizing the promoter for the human UCHL1 gene.

Embodiment 22. The vector of any of the embodiments 14-21, wherein the vector comprises the UCHL1 promoter, operably linked to a nucleic acid sequence encoding a therapeutic molecule (e.g., a polypeptide or nucleic acid such as an RNA).

Embodiment 23. A pharmaceutical composition comprising: (i) the vector of any of embodiments 14-22; and (ii) a carrier, excipient, or diluent.

### EXAMPLES

The following Examples are illustrative and should not be interpreted to limit the scope of the claimed subject matter.

### Example 1—Compositions and Methods for Treating Neurological Diseases, Disorders, and Injuries Associated with the Activity of Upper Motor Neurons

Overview

Neurodegenerative diseases are characterized by progressive degeneration of certain neuron populations, while others remain relatively healthy. This is called “selective vulnerability”. Upper motor neurons display selective vulnerability and undergo progressive degeneration in diseases that are characterized by the loss of voluntary movement and motor neuron dysfunction. These diseases are deadly, and there are currently no affective treatment strategies. Upper motor neuron death is also one of the major reasons for long-term paralysis in spinal cord injury patients. Therefore, improving the health and the connectivity of upper motor neurons in patients have important clinical implications. These neurons degenerate in hereditary spastic paraplegia, primary lateral sclerosis, amyotrophic lateral sclerosis patients as well as in patients of diseases in which voluntary movement and motor neuron circuitry is affected, such as Parkinson's and multisystem motor degeneration.

Even though a large percentage of neurodegenerative diseases develop due to “unknown” causes and are considered to be “sporadic”, the genetic basis of neurodegenerative diseases are beginning to emerge at a very fast pace. Therefore, it is important to develop therapies that are geared towards repairing the genetic defect that leads to upper motor neuron degeneration in patients.

Technical Description. We have developed a gene delivery approach to transduce upper motor neurons (UMN) specifically using the AAV2 skeleton and human UCHL1 promoter. As proof of concept, we developed and sequenced AAV2-hUCHL1-mCherry to assess upper motor neuron transduction. Our preliminary data suggest higher levels of UMN a.k.a. corticospinal motor neurons (CSMN) transduction upon direct injection of the AAV into the cerebral cortex.

The injection requires surgery into the motor cortex. The motor cortex area that harbors upper motor neurons that project to the cervical and lumbar regions need to be targeted. The size and the location of the area differs among species and for humans it lies mainly within the Brodmann area 4 of the cerebral cortex. The virus needs to be delivered (the titer and the speed may need to optimized based on the patient, but more broadly 3×10E-9-5×10E-13 viral particles per microliter, and 20 nanoliter per minute speed and a total volume of 5-10 microliters for 4-5 independent injection sites that span the motor cortex and aims at the layer V of the motor cortex, at a depth of about 1.56-1.86 mm in humans). These surgeries will be performed by neurosurgeons, and the location as well as the taxonomy of the motor cortex is well established in humans. These surgeries are rather non-invasive and the patient may not even need to stay overnight at the hospital. Compared to deep-brain stimulations of Parkinson's patients, in which the probe has to cross the whole depth of the cerebral cortex to reach substantia nigra pars compacta, these motor cortex injections are rather safe and low-risk injections. Also it is important to note that these are one-time injections. Patients do not need to be prepped for a second injection. One time injection should induce low but steady levels of gene expression in the neurons of interest.

A. Directed Gene Delivery to Upper Motor Neurons in Macaque Monkeys

Utilization of viruses for gene delivery is not new. However, currently, gene delivery approaches are very broad, transducing a very broad spectrum of cells and neurons in the central nervous system. This would induce problems because not all cells and neurons require genetic correction to the same level and extent. Because there is selective vulnerability, there also needs to be a selective transduction only to the neurons in need, without affecting other neurons and cells in the brain and in the body. Therefore, we propose that utilization of correct promoters to target the neurons of interest is very important for directed gene delivery and for building effective and safe treatment strategies in the near future. If neuronal vulnerability is selective, the treatment strategy should also be selective.

We initially developed approaches to transduce upper motor neurons using AAV. We initially used retrograde transduction by injecting AAV to the corticospinal tract that allows viruses to move retrogradely towards the brain, and transduce the upper motor neurons selectively. We found that AAV2 serotype was one of the best serotypes for retrograde transduction. We also performed the same surgeries in ALS mouse models and obtained similar levels of transduction, suggesting that AAV would transduce diseased neurons as well, opening doors for future therapeutic interventions, (see e.g., Jara et al., AAV2 mediated retrograde transduction of corticospinal motor neurons reveal initial and selective apical dendrite degeneration in ALS, Neurobiol Dis. 2012 August; 47(2); 174-183, incorporated herein by reference in its entirety).

Retrograde transduction is not an approach favored in human patients because the corticospinal tract is not located in the dorsal funiculus in humans, rather the corticospinal tract is close to the spinal motor neurons in the ventral horn of the spinal cord. Direct injection into the corticospinal tract in human patients would be very challenging and a limiting factor. Therefore, a better approach would be to develop direct cortex injections; the cortex is more accessible, and the procedure is less invasive and more easily tolerated. This approach is very straightforward and it is much easier than some of the FDA approved cortical surgeries performed on Parkinson patients that undergo deep brain stimulation. Therefore, for more translational efforts direct motor cortex injection strategies need to be developed. The limiting factor in these surgeries is the complexity of the cerebral cortex with thousands of different neuron and non-neuronal cells located in close proximity to each other and viral transductions that are non-specific with respect to a given cell or neuron population, and therefore affecting many untargeted cells and neurons. We think this is a major problem especially in the long-run because as we hope to improve the health of distinct neuron populations, there will inevitably be many unwanted, off-target effects; many other cells and neurons could be badly affected. Therefore, a strategy that is neuron specific and selective is needed.

We thus developed strategies to target upper motor neurons by one-time direct motor cortex injection. The success of this experiment depends on the serotype of the virus to be used and the choice of promoter. We tried AAV serotypes 1, 2, 3, 4, 5, 6, 7, 8, 9 and found that AAV2 transduces mostly neurons. We then tried different promoters, such as CMV and CBA and found that upon the use of CMV promoter to drive the gene of interest, the transduction efficiency of CSMN (corticospinal motor neurons, the upper motor neurons in mice) reaches up to 70%, which means among all cells and neurons that are transduced, 70% are CSMN. If one considers that CSMN make up less than 1% of the motor cortex, and if upon one single transduction 70% of the cells/neurons are CSMN, which means transduction favors CSMN 70 times, which is remarkable. We also found that 20% of the other neurons that are transduced are callosal projection neurons and this is also important because callosal projection neurons are secondary neurons that degenerate in diseases, such as ALS. Especially during end-stages of the diseases there is callosal neuron degeneration and patients begin to display cognitive decline as well. Therefore, targeting a small population of callosal projection is also advantageous. Since upper motor neurons become diseased, one potential problem is that they will lose their ability to be transduced via viruses. Therefore, surgeries are performed at different stages of the disease in ALS mouse model to investigate whether diseased upper motor neurons can be transduced and whether there is a critical time window.

We thus tried two different times of transduction and our results revealed that diseased neurons were transduced even when disease symptoms were initiated. This was rather important as it opened a window of opportunity for neurons to be transduced during and after symptom onset. This is an important finding because it reveals that directed gene delivery to diseased upper motor neurons can also be performed during symptomatic stages of the disease, a time when patients seek medical attention. This finding allows our strategies to be adopted by sporadic ALS patients, which comprise about 90% of all ALS patients.

Even though 70% CSMN transduction is very high, we sought to improve our specificity towards upper motor neurons, and therefore tested different promoters. We discovered that with the use of UCHL1 promoter, the transduction efficiency is very low, but the neurons that are transduced are mainly the upper motor neurons. These studies are still ongoing both in vitro and in vivo, but our current results suggest about 97-99% specificity towards upper motor neurons. We used the human UCHL1 promoter to drive mCherry so that we can visualize the neurons that are transduced with a red fluorescence.

The other consideration with gene delivery via viruses is whether it is possible to induce novel gene expression in distinct neuron populations. Accordingly, we investigated this by expressing UCHL1 in the UCHL1 null mice, which do not have endogeneous UCHL1. Thus if viral transduction works and gene expression is complete then a full protein should be detected by UCHL1 immunoassay. These experiments were also successful and we were able to deliver the UCHL1 gene directly into the upper motor neurons and only the upper motor neurons in the UCHL1 null mice, and the transduced neurons began to express UCHL1 protein. Accordingly, gene delivery via virus to distinct neuron population was a success.

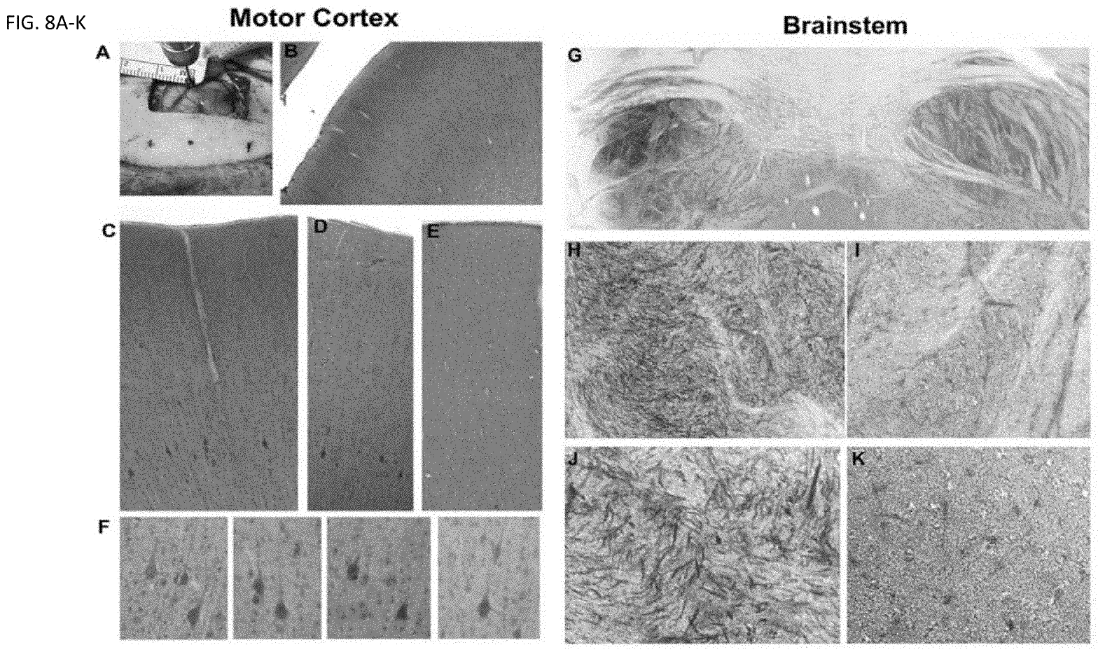

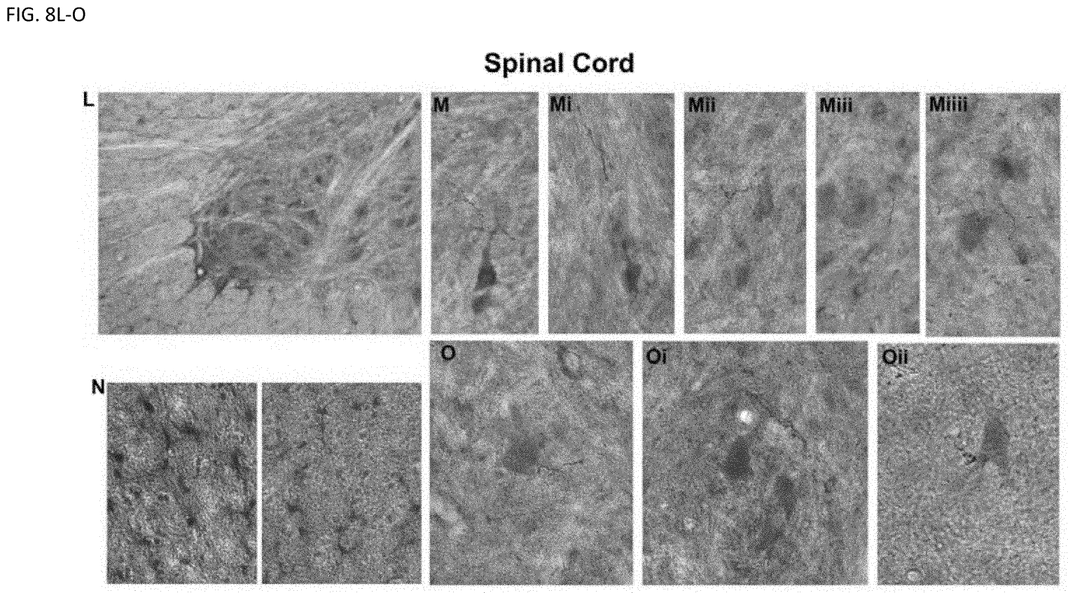

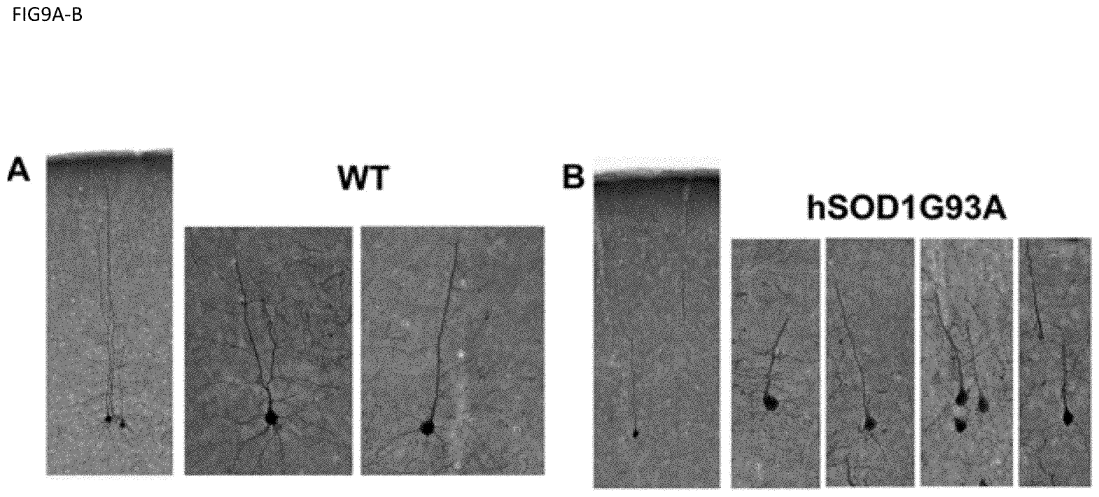

More importantly, we began to perform surgeries on the motor cortex of Macaque monkeys, whose motor cortex highly resembles human motor cortex and they are one of the most closely linked species to human when it comes to motor neuron circuitry. These surgeries targeted the Betz cells in the monkey (n=2). Betz cells have monosynaptic connections with the spinal motor neurons and are located in brain regions that reciprocate to the Brodmann area 4 in humans. 69 nl litters of AAV2-hUCHL1-Ires-mCherry was injected into 2.2 mm and 2 mm from the surface of the cerebral cortex per site and 4 different sites were targeted with injection. FIG. 8A-D. These surgeries revealed the presence of transduced Betz cells, as evidenced by their prominent apical dendrites and their soma. These cellular structures were visualized with tissue clearance as it is not possible to have the soma and the full extent of the apical dendrite in the same 50 micrometer section plane. FIG. 8E-K. The spinal cord was sectioned and further investigated to visualize corticospinal tract axons and their connections with the spinal motor neurons. (FIG. 8L) The ventral horn of the spinal cord is where the spinal motor neurons reside and numerous axon fibers entering the ventral horn were detected. (FIG. 8M) Numerous examples of upper motor neuron axon fibers located in close proximity to spinal motor neurons were noted. (FIG. 8N) The cross section of the anterior corticospinal tract within the spinal cord display dense axon fibers crossing into the spinal cord. The contralateral side is does not have axon fibers. (FIG. 8O) Detailed investigation of axon fibers also reveal direct connections of the upper motor neuron axons with spinal motor neurons. In some cases, the growth cones and the site of connectivity is visualized, further confirming the identity of neurons that are transduced to be upper motor neurons. Surgeries were well tolerated and monkeys did not have problems recovering. Therefore, we believe that directed gene delivery to patients' Betz cells with one-time virus injection will be well tolerated and will result in life-long benefits for improving and maintaining the good health of upper motor neurons.

### DISCUSSION

We previously worked in the mouse models to find the promoters that are more selective to upper motor neurons.

We now worked with Macaque monkeys and injected AAV into the motor cortex. The AAV had the expression vector which used human UCHL1 promoter to drive the expression of mCherry, We found that not all cortical neurons/cells are transduced. Rather, the transduction is very specific to the upper motor neurons that are located in layer V of the motor cortex. We further proved that the transduced neurons are indeed upper motor neurons by tracing the axonal fibers in the brainstem and in the spinal cord. We found very selective and high level transduction of upper motor neurons as their axonal projections were detected in the medullary pyramid within then brainstem, and in the lateral corticospinal tract within the spinal cord. In addition, we detect axon fibers in close proximity to the spinal motor neurons within the ventral horn, some even making direct connections. Therefore, we are confident that the neurons transduced are indeed upper motor neurons. The transduction is also very specific, no other cortical neurons are transduced and we do not detect any astrocyte or micgroglia transduction. This is very specific to upper motor neurons. We have performed two independent experiments with two Macaque monkeys and both gave the exact same results.

Giving UCHL1 to diseased neurons will improve their heath so introduction of UCHL1 expression is a treatment strategy for upper motor neurons.

Using the UCHL1 promoter, one can target upper motor neurons with specificity. This is important for future gene therapy approaches, where a gene of interest can be given to the upper motor neurons of patients who suffer diseases that result due to progressive degeneration of upper motor neurons, such as hereditary spastic paraplegia, primary lateral sclerosis, amyotrophic lateral sclerosis and even the spinal cord injury patients, who develop long-term paralysis due to degeneration of upper motor neurons after injury.

This is an approach that can be tailored towards the needs of each patient. For example, if a patient is known to have a known mutation, the wild type and functional form of the gene can be cloned under the control of UCHL1 promoter and applied to the motor cortex of the patient. Alternatively, if a cellular function is determined to be affected, the gene that codes for enzymes or modulators of that cellular function can be cloned into the expression system. In some cases, some patients may have over expression of a given gene and this may be one of the causes of neuronal vulnerability, in these cases the shRNA of the gene product can be cloned into the expression system such that the levels of the protein product will be lowered specifically in the neurons of interest, without affecting other cells and/or neurons. Overall transduction efficiency with UCHL1 promoter is rather low (about 24% of all cell/neurons in the vicinity are transduced) and this is rather important for not having off target affects. However, among all cells and neurons transduced, the upper motor neurons make up about 95-97%, which is remarkably specific.

Therefore, especially for diseases that affect upper motor neurons, the compositions and methods of directed gene delivery disclosed herein will offer an effective solution, and the use of hUCHL1 promoter will help gene delivery efforts to target the neurons in need, without affecting other cells and neurons in the cerebral cortex.

B. Importance of UCHL1 for Upper Motor Neurons and UCHL1 as a Target for Neuronal Improvement: Therapeutic Effects in ALS Mouse Models.

We initially found that UCHL1 expression is stable in the upper motor neurons and that upper motor neurons begin to express UCHL1 very early in their development and continue to express the gene throughout life. The stable gene expression of UCHL1 within upper motor neurons, inspired us to generate the UCHL1-eGFP mice in which the upper motor neurons are genetically labeled with eGFP expression that is stable and long-lasting. We generated and characterized UCHL1-eGFP mice and showed that eGFP expression is restricted to the upper motor neurons in the motor cortex by numerous levels of analysis: 1) retrograde labeling; 2) molecular marker expression; 3) electrophysiology; 4) connectivity studies; 5) generation of disease models in which upper motor neurons degenerate and confirming that these eGFP neurons are indeed the ones that selectively degenerate (see e.g., Yasvoina et al., eGFP Expression under UCHL1 Promoter Generically Labels Corticospinal Motor Neurons and Subpopulation of Degeneration-Resistant Spinal Motor Neurons in an ALS Mouse Model, Journal of Neuroscience, 2013; 33(18) 7890-7904).

We then reasoned that if UCHL1 expression is present in upper motor neurons all through life, then its absence would affect the health of upper motor neurons. We studied upper motor neuron health in mouse models that lack UCHL1 function and found that upper motor neurons undergo profound degeneration that starts very early. There is immense apical dendrite disintegration in upper motor neurons as they display progressive neuronal degeneration. This further proved the importance of UCHL1 for the upper motor neurons.

We also found that the same cellular pathology observed in upper motor neurons of mouse models are also present in the Betz cells of ALS patients with a broad spectrum (sALS, fALS, and ALS/FTLD). Betz cells in patients also display exactly the same cellular degeneration with apical dendrite degeneration and spine loss. Even though patients develop the disease due to very many different underlying causes, the cellular pathology was exactly the same as the one observed in the upper motor neurons of mouse model that lacked UCHL1 function, further suggesting a significant role UCHL1 plays in the health and integrity of upper motor neurons. We published the extent of apical dendrite degeneration in ALS patients (see e.g., Genç, el al. Apical dendrite degeneration, a novel cellular pathology for Betz cells in ALS. Sci Rep., 2017; 7, 41765; incorporated herein by reference in its entirety). These findings are important as they reveal that the pathology at a cellular level is translational and if we promote the health of the upper motor neurons at a cellular level, patients will benefit from this improvement.

Because lack of UCHL1 function resulted in profound upper motor neuron degeneration without apparent spinal motor neuron loss, which we reported at Annals of Clinical and Translational Neurology 2016, we reasoned that UCHL1 function is particularly important for upper motor neuron health and addition of UCHL1 to the upper motor neurons that degenerate in the UCHL1 null mice would improve their health. Since all neurons and cells lacked UCHL1 function in the UCHL1 null mice, it was important to determine whether upper motor neuron degeneration was due to intrinsic or extrinsic factors. If upper motor neuron degeneration was due to extrinsic factors, than introduction of UCHL1 to the upper motor neurons would not be sufficient to improve their health, but if upper motor neuron death was due to intrinsic factors, specific replacement of UCHL1 in the upper motor neurons would be sufficient to improve their health. Therefore, we needed to develop ways to introduce UCHL1 in the upper motor neurons. We used AAV-mediated gene delivery approaches and injected the AAV2 virus, which encodes UCHL1 gene in the motor cortex of UCHL1 null mice. We found that introduction of UCHL1 into the upper motor neurons of UCHL1 null mice, was sufficient to improve the cellular integrity of upper motor neurons and their neuronal health. This is an important finding as it further proves that UCHL1 function is intrinsically important for the upper motor neurons and their progressive degeneration in the UCHL1 null mice is not a secondary event or a function of neuron loss in other regions of the CNS. Therefore, introduction of UCHL1 in diseased upper motor neurons could have therapeutic implications for neurodegenerative diseases in which upper motor neurons are affected.

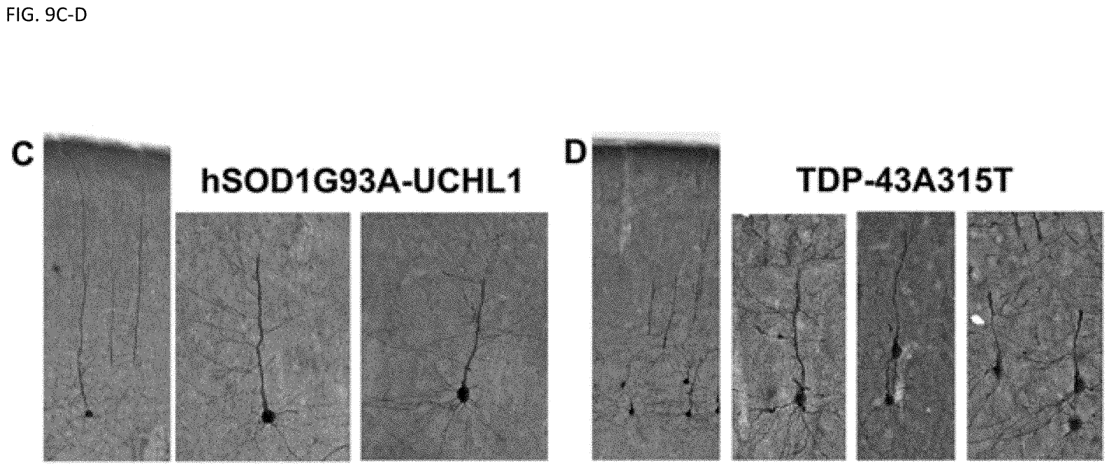

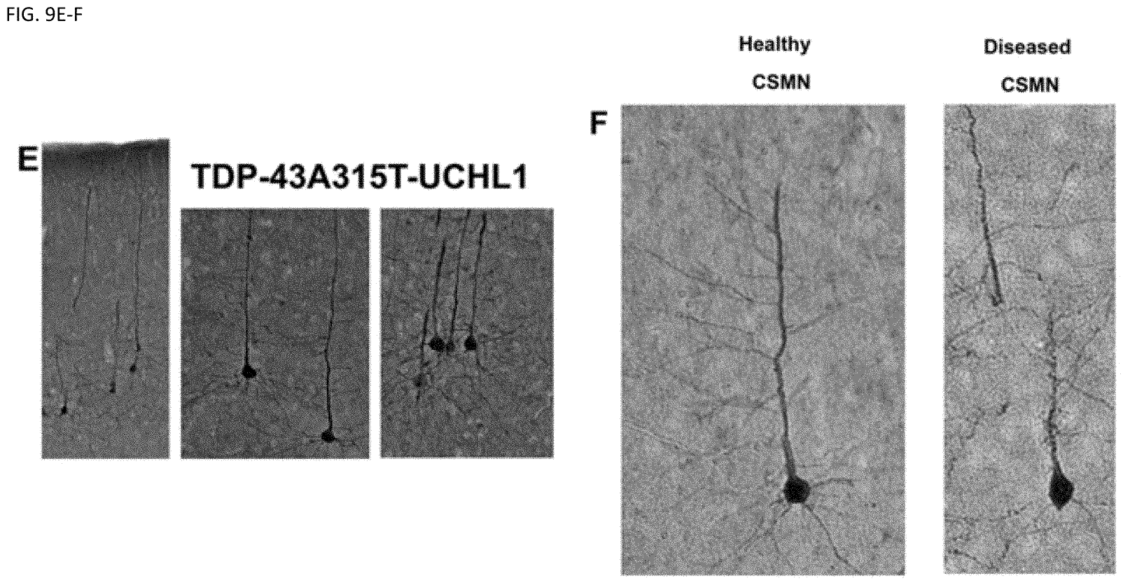

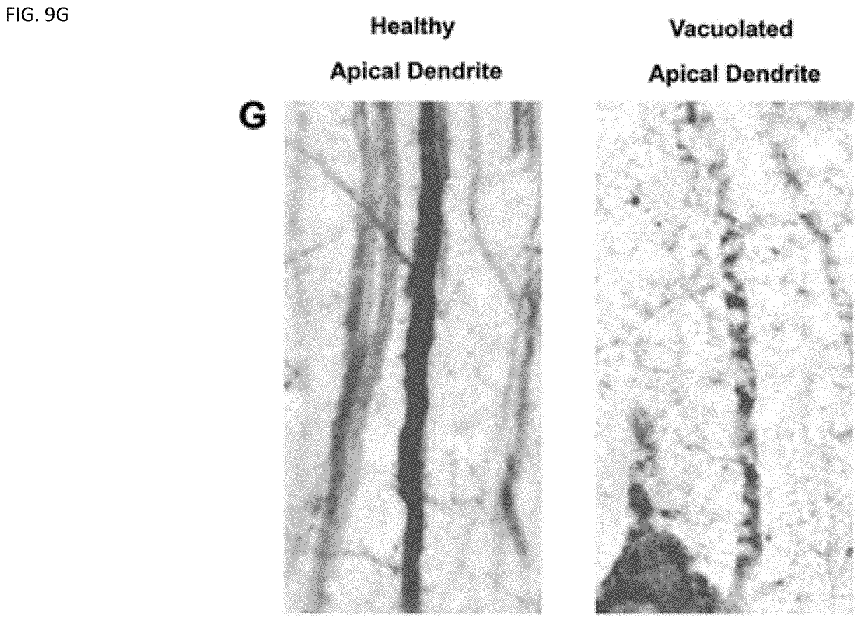

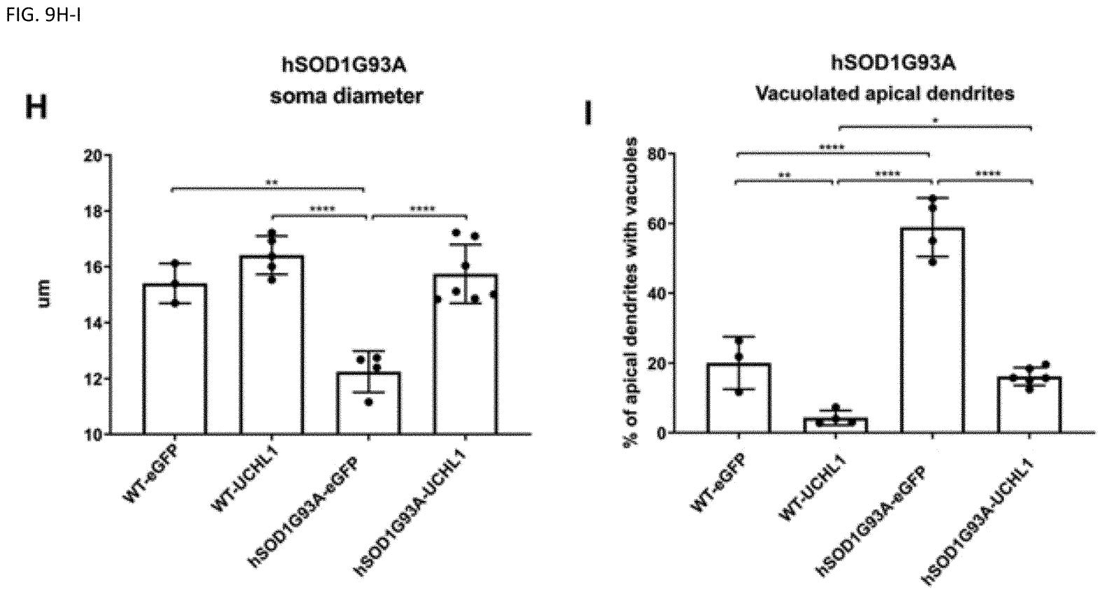

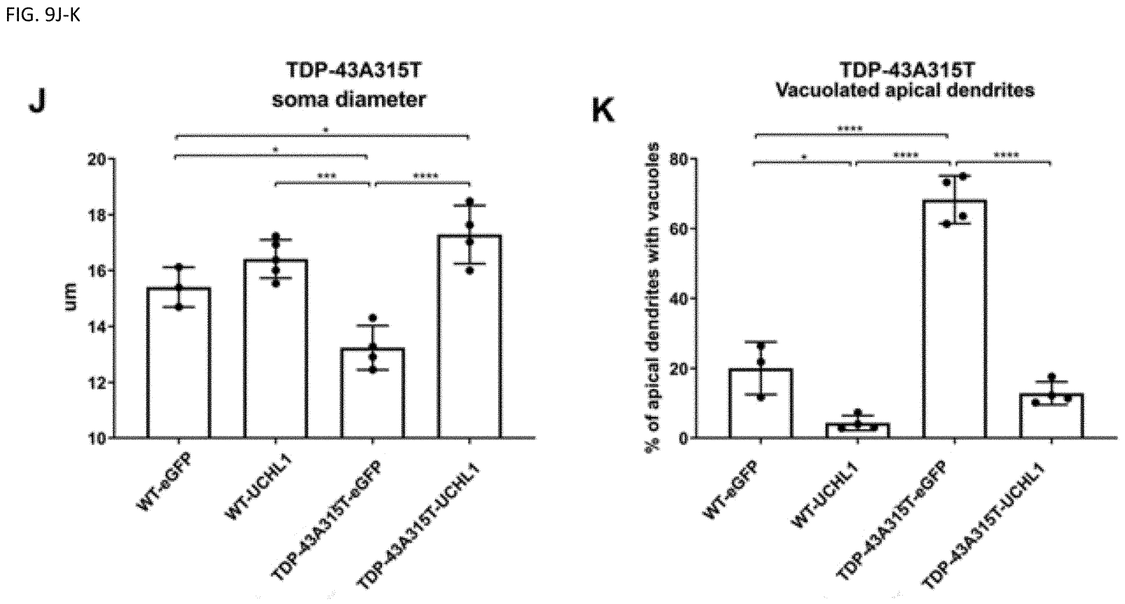

To further test the hypothesis that introduction of UCHL1 to diseased neurons will improve their cytoarchitectural integrity and health, we introduced UCHL1 gene in to the motor cortex of hSOD1^{G93A} mice, a well-studied mouse model of ALS and in a TDP-43 mouse model, which has the A315T mutation, a mutation detected in ALS patients. The upper motor neurons degenerate in the SOD1 mouse model is due to mSOD1 toxicity and not due to lack of UCHL1 function. Therefore, it was important to test whether, introduction of UCHL1 to an upper motor neuron that degenerates due to a different cause would also lead to improved neuron health. Our experiments revealed that introduction of UCHL1 to the CSMN of hSOD1^{G93A} mice and TDP-43 mice improved the cytoarchitectural integrity, stability and health of CSMN. See FIG. 9A-K.

TDP-43 model: TDP-43 pathology is one of the most common proteinopathies (Neumann, Sanpathu et al. 2006, Bigio 2013, Cykowski, Powell et al. 2017, Heyburn and Moussa 2017) and the TDP-43 models generated to recapitulate human pathology have motor function detects and motor neuron degeneration (Wegorzewska, Bell et al. 2009, Wegorzewska and Baloh 2011). Some of the major mechanisms that contribute to degeneration are nucleus to cytoplasm transport defects, reduced mitochondrial health, ER stress, neuroimmune modulation and protein accumulation (Wang, Li et al. 2013, Wang, Wang et al. 2016, Chou, Zhang et al. 2018, Davis, Itaman et al. 2018, Gautam, Jara et al. 2019, Gautam, Xie et al. 2019).

SOD1 model: Since the SOD1 mutation was one of the first mutations identified and the SOD mouse model is extensively studied (Gurney, Pu et al. 1994, Julien and Kriz 2006, Philips and Rothstein 2015), we know more about the cellular events that are perturbed with respect to SOD1. For example, oxidative stress, ER stress, protein aggregation defects, problems with mitochondria, axon transport defects, astrogliosis microgliosis, neuroimmune modulations are the most significant mechanisms that contribute to motor neuron degeneration (Pasinelli, Belford et al. 2004, Fischer and Glass 2010, Li, Vande Velde et al 2010, Pickles, Destroismaisons et al 2013, Pollari, Goldsteins et al. 2014, Tan, Pasinelli et al. 2014, Pickles, Semmler et al. 2016, Kim and Taylor 2017, Bakavayev, Chetiit et al 20191 However in patients with SOD1 mutations, TDP-43 accumulations are not detected (Mackenzie, Bigio et al. 2007, Robertson, Sanelli et al. 2007).

We performed retrograde transduction to ensure all neurons that are transduced are upper motor neurons in the motor cortex.

We used hSOD1G93A mice, which represents the model that mimics disease pathology, which occurs due to misfolded SOD1 toxicity and this mouse model is well-characterized for ALS. We previously showed progressive CSMN loss in these mice and therefore, we expressed UCHL1 in upper motor neurons of these mice to investigate whether UCHL1 would improve upper motor neuron health.

We also used a TDP-43 mouse model, which has the A315T mutation, a mutation detected in ALS patients. This mouse was developed to mimic TDP-43 pathology observed in ALS patients and ALS/FTLD patients. Importantly, we worked with this mouse model in depth and found that that the upper motor neurons undergo progressive degeneration, and the underlying cellular causes for their upper motor neuron degeneration is exactly same with then underlying causes of degeneration that occur in the tipper motor neurons of patients with TDP-43 pathology. There is immediate translation between upper motor neurons at a cellular level. Therefore, being able to improve the health of upper motor neurons in the TDP-43 mouse model would have direct translation to the upper motor neurons of patients who have TDP-43 pathology. This is important because about 75-80% of ALS patients have TDP-43 pathology and almost all ALS/FTLD patients have TDP-43 pathology.

When we express UCHL1 in the upper motor neurons of TDP-43Aβ I5T mice, we observe highly significant improvement of their health, such that their soma size becomes comparable to that of WT healthy upper motor neuron, and the vacuolization of apical dendrite is significantly reduced to the levels of healthy controls.

So we now have proof (see e.g., FIG. 9A-K) with two independent mouse models that expression of UCHL1 is highly beneficial and it reverses neuronal degeneration. UCHL1 is indeed a good target to improve the health of diseased upper motor neurons.

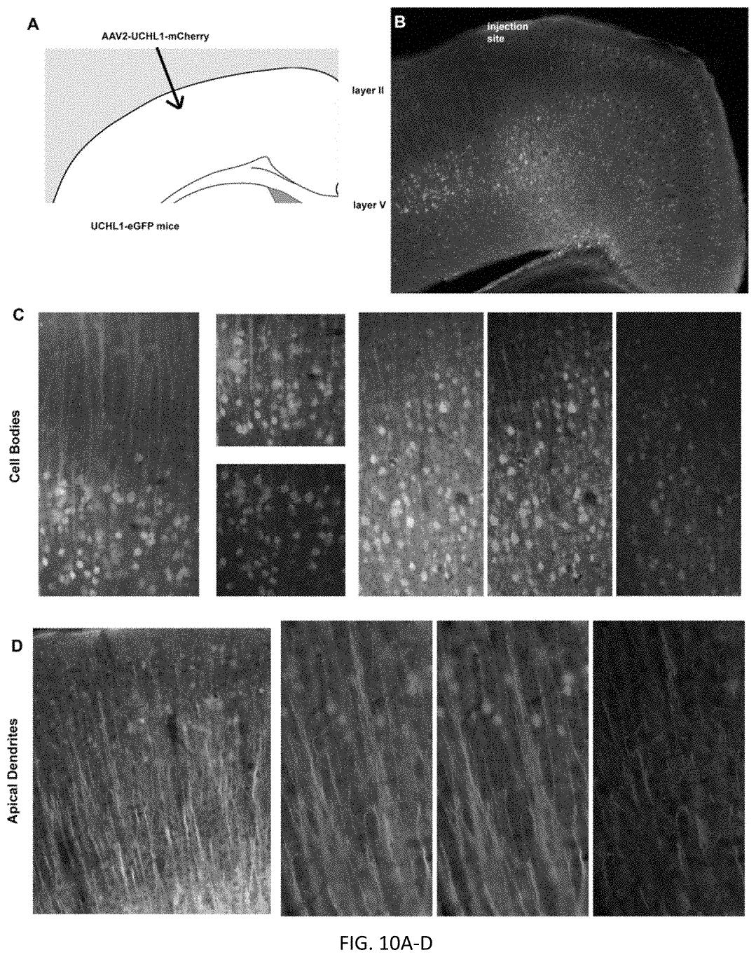

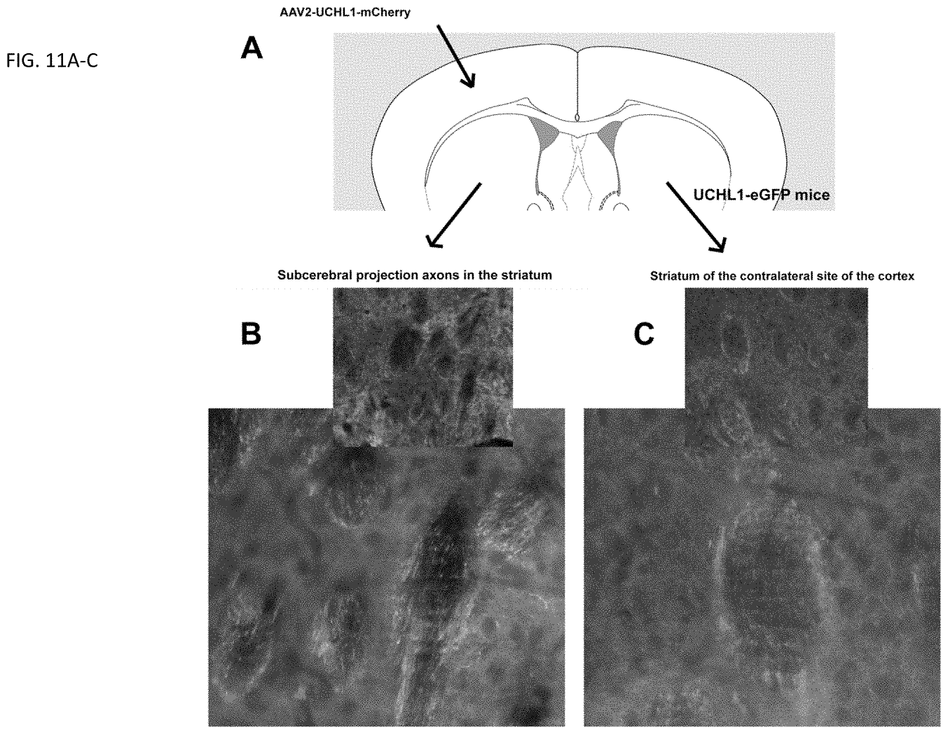

Finally FIG. 10A-D and FIG. 11A-C show additional experiments in the UCHL-EGFP mice, illustrating the specificity of the UCHL1 promoter to direct expression to upper motor neurons. The UCHL-EGFP mice were given direct motor cortex injection of AAV2-UCHL1-mCherry. As show in the figures, the co-localized EGFP and mCherry indicates specific targeting. Additionally, mCherry labeled axon terminals in the striatum of the injected side further demonstrate the specificity of the UCHL1 promoter.

The use of UCHL1 promoter in AAV-mediated transduction targets mainly the large pyramidal subcerebral projection neurons, including corticospinal motor neurons, the upper motor neurons, that degenerate in upper motor neuron diseases, such as ALS (amyotrophic lateral sclerosis), HSP (hereditary spastic paraplegia), PLS (primary lateral sclerosis).

Upon direct motor cortex injection of AAV2-UCHL1-mCherry, astrocytes and microglia are not transduced (0%, n=300; 300 transduced neurons counted and none of them were astrocyte or microglia), and transduction is very restricted to upper motor neurons located in layer V of the motor cortex. Transduction efficiency is much better than CMV and CBA promoters and is more specific to upper motor neurons (98.5% for UCHL1 promoter (n=250 neurons counted in n=2 mice).

Discussion

We think these findings are very important as they suggest that introduction of UCHL1 to diseased upper motor neurons would have therapeutic implications in a broad spectrum of patients. Moreover, the results surprisingly and unexpectedly show the specificity of the UCHL1 promoter for targeted gene expression in the upper motor neurons.

Direct cortex injection surgeries were performed in both mice and in macaque monkeys. Despite species differences, the use of UCHL1 promoter to drive mCherry expression led to the transduction of upper motor neurons located in layer V of the motor cortex. The specificity was more obvious in macaque monkey, as transduction very specifically labeled large upper motor neurons in layer V of the motor cortex. The upper motor neuron identity of these transduced neurons are further confirmed by the presence of their axons in the brainstem and in the spinal cord, making connections with the spinal motor neurons.

Therefore, we propose that the use of the UCHL1 promoter in expression vectors will help target upper motor neurons in both in vitro and in vivo experiments. And importantly, the direct gene delivery approaches will benefit patients who suffer from diseases that affect the health of the upper motor neurons, and injuries that impair the health of their upper motor neurons. Any patient who display defects in their voluntary movement would benefit from direct gene delivery to their Betz cells.

We also propose that UCHL1 protein is important for the health and the function of upper motor neurons and introduction of UCHL1 to diseased upper motor neurons improve their cytoarchitectural integrity, and health. Therefore, we propose to use UCHL1 in gene delivery approaches to upper motor neurons that are diseased and that are injured.

Most preclinical in vivo studies utilize mouse models that are generated by the mutation or pathology detected in patients, and that closely recapitulate many of the human pathology (Janus and Welzl 2010, Thomsen, Gowing et al. 2014, Haston and Finkbeiner 2016, Dawson, Golde et al. 2018, Hawrot, Imhof et al. 2020). So far, the extension of life span in mouse models is considered the “gold standard” for assessing the success of screened compounds, but unfortunately this assumption fails to translate to improved survival in patients (2013, Perrin 2014, Ransohoff 2018). Even though motor neuron diseases develop because motor neurons degenerate, there has never been a study that investigates the health and betterment of diseased UMNs. One of the major limitations has been the lack of proper tools and drug discovery platforms that would utilize UMN response as the readout. In their absence, preclinical assays rely heavily on extension of the life span of mouse models as the outcome measure (Gurney 1997, Janus and Welzl 2010, Ahmed, Irish et al. 2017, Lutz 2018, De Giorgio, Maduro et al. 2019, Fisher and Bannerman 2019). However, the lack of translation from mouse models to humans has resulted in numerous failed clinical trials and compounded frustrations. The need to develop better preclinical assays that provide information about the survival needs of vulnerable and degenerating neurons has become evident (Genc and Ozdinler 2014, Ozdinler and Silverman 2014, Dervishi and Ozdinler 2018). Focusing our attention to the diseased neuron at a cellular level generates information that is translational (Mackenzie, Bigio et al. 2007, Robertson, Sanelli et al. 2007, Dervishi and Ozdinler 2018, Gautam, Jara et al. 2019, Genc, Gozutok et al. 2019). Comparing mice to human may not be feasible, and extension of life-span in mice may not translate to extension of survival in ALS patients, but the motor neurons in mice and the motor neurons in humans are almost identical at a cellular level and they share the same aspects of neuronal pathology. For example, motor neurons in patients with TDP-43 pathology become vulnerable and undergo degeneration due to nucleocytoplasmic, mitochondrial and ER defects. The same pathology is present in the motor neurons of TDP43 mouse model (Gautam, Jara et al. 2019). Therefore, in an effort to reveal translational information we must obtain data exactly from neurons that display selective vulnerability to diseases.

Because misfolded SOD1 toxicity and TDP-43 pathology represent two distinct, and mostly non-overlapping causes of ALS, being able to identify a compound that improves the health and stability of UMNs that become diseased due to these two causes would have implications in a broad spectrum, including ALS, HSP, PLS, ALS/FTLD, and FTLD patients. UMN degeneration is a prominent feature of motor neuron diseases in which voluntary movement is impaired. Mouse models recapitulating many aspects of motor neuron diseases are generated, such as the hSOD1^{G93A} and the prpTDP-43^{A315T} mice. The timing and the extent of UMN loss have been reported in these mouse models (Ozdinler, Benn et al. 2011, Yasvoina, Genc et al. 2013, Gautam, Jara et al. 2019), which were developed to recapitulate and understand SOD1 toxicity and TDP-43 pathology mediated motor neuron degeneration, respectively (Gurney, Pu et al. 1994, Wegorzewska, Bell et al. 2009). Mounting experimental data now reveal that the cellular pathology of UMNs becomes evident much earlier than symptom onset (Jara, Villa et al. 2012, Yasvoina, Genc et al. 2013, Jara, Genc et al. 2015, Jara, Gautam et al. 2019). Spine loss and apical dendrite degeneration occurs prior to neuronal loss (Fogarty, Noakes et al. 2015, Fogarty, Klenowski et al. 2016, Fogarty, Mu et al. 2016, Handley, Pitman et al. 2017), and cortical hyperexcitation is used as an early detection marker of ALS. (Vucic, Ziemann et al. 2013, Menon, Geevasinga et al. 2015, Geevasinga, Menon et al. 2016, Huynh, Simon et al. 2016). The apical dendrite degeneration observed in UMNs that become diseased as a result of mSOD1 toxicity (Jara, Villa et al. 2012, Yasvoina, Genc et al. 2013), lack of Alsin function (Gautam, Jara et al. 2016), Profilin mutations (Fil, DeLoach et al. 2017) and TDP-43 pathology (Jara, Gautam et al. 2019) was also recapitulated in the Betz cells of fALS, sALS, and ALS with FTLD patients (Genc, Jara et al. 2017). Therefore, being able to reverse the ongoing apical dendrite degeneration would have significant outcomes for neuronal health and connectivity.

Mutations in the SOD1 gene were identified in ALS patients (Rosen, Siddique et al. 1993), and the disease mouse models that evolved were based on the mutations detected in patients (Gurney, Pu et al. 1994); these models mimicked many aspects of human pathology, including progressive UMN loss (Ozdinler and Macklis 2006, Yasvoina, Genc et al. 2013). TDP-43 pathology, on the other hand, develops regardless of a mutation in the TARDP gene (Coan and Mitchell 2015, Cykowski, Powell et al. 2017), and it is observed in the brains of about 95% of ALS patients (Ling, Polymenidou et al. 2013). Most patients with SOD1 mutations do not display TDP-43 pathology in their brains (Mackenzie, Bigio et al. 2007, Robertson, Sanelli et al. 2007); therefore, mSOD1 and TDP-43 seem to represent two different and non-overlapping pathologies. Therefore, identification of a compound that improves the health of UMNs that become diseased by these two prominent and distinct causes, is rather significant.

AAV2-GFP mediated retrograde labeling of CSMN (Jara, Villa et al. 2012, Jara, Genc et al. 2014) allows detailed visualization and assessment of CSMN health and integrity, especially at the site of the apical dendrite. In order to test the ability of UCHL1 expression to improve the health of CSMN that become diseased due to mSOD1 toxicity and TDP-43 pathology, two independent and overarching causes of neurodegeneration, we applied AAV2-mediated delivery of the UCHL1 targeted only to CSMN via retrograde surgery.

Materials and Methods

pGEM-T vector plasmid containing the mouse UCHL1 cDNA ORF clone was purchased from Sino Biological (cat: MG50690-G, Wayne, Pa.), and the UCHL1 CDS was subcloned into a AAV plasmid with CBA promoter (Zhu, Xu et al. 2014) to generate pAAV.CBA.UCHL1-IRES-eGFP.WPRE plasmid that was packaged into AAV2 virus particles by the University of Pennsylvania Vector Core facility.

Direct Motor Cortex Injection Surgeries:

Mice:

Surgeries were performed on mice that were deeply anesthetized with isoflurane and placed into a stereotaxic platform. Micro-injections were performed using pulled-beveled glass micro-pipettes attached to a nanojector (Drummond Scientific, Broomall, Pa., USA).

To inject the different serotypes into the motor cortex, a small unilateral craniotomy of ˜5 mm² (coordinates=+0.5 mm anterior-posterior; 1.5 mm mediolateral) was performed into the left hemisphere using a microdrill (Fine Science Tools, Foster City, Calif., USA). Ten injections of 64 nl were performed to target the primary and secondary motor cortex for a total of 640 nl containing 1.2×10⁹ transducing units per mouse.

Monkey:

The injection of AAV is performed under anesthesia in a surgical room with a fume hood specifically designed for viral injections. The injections site was in the arm/hand area of primary motor cortex either on the precentral gyrus or anterior bank of the central sulcus so as to target corticospinal motor neurons. Endpoints: approximately 8-12 weeks after AAV injection the animal are euthanized, and the tissue is harvested.

Pre-operative preparation: On the day of surgery the animal is food restricted for 12-14 hours before the procedure. The animals are squeezed in their cages and sedated with Ketamine or Ketamine/Dexmedetomidine. The animal is then intubated and anesthesia induced and maintained using inhalant isoflurane. The hair over the surgical sites is clipped.

Surgical procedure: The injection of AAV is performed under anesthesia in a surgical room with a fume hood specifically designed for viral injections. The injections site will be in the arm/hand area of primary motor cortex either on the precentral gyrus or anterior bank of the central sulcus so as to target cortimotoneuronal neurons. The macaques are placed in a Kopf sterotaxic apparatus and the surgical site is sterilized. An incision is made over the area of injection. A bur is used to make a small craniotomy in the skull to see relative landmarks in the cortex. Previously tittered viral vector concentrate is stereotaxically injected into the cortex using a 25-27G hamilton syringe attached to an electric infusion syringe pump (Steolting). Once the injection is completed, bone flap is replaced and secured with a titanium strap. Muscle and skin are sutured with nylon, vycryl suture over the area of injection. Sutures are removed prior to euthanasia.

Retrograde Transduction.

Surgeries were performed on a stereotaxic platform. Micro-injections were performed using pulled-beveled glass micro-pipettes attached to a nanojector (Drummond Scientific, Broomall, Pa.). CSMN were retrogradely labeled either by pAAV.CBA.UCHL1-IRES-eGFP.WPRE or by pAAV.eGFP empty control vector (621 nl containing 1.16×10⁹ viral particles), injected into the CST at P60 and CSMN were retrogradely transduced as described (Jara, Villa et al. 2012, Jara, Genc et al. 2015). At P120, mice were deeply anesthetized with ketamine (90 mg/kg) and xylazine (10 mg/kg) and perfused with 4% PFA in PBS.

The brain was removed intact from each mouse, post-fixed by 4% PFA overnight and kept in PBS-sodium azide (0.01%) at 4° C. Brains were sectioned (coronal; 50 m) using a vibrating microtome (VT1000SLeica Instruments, Nussloch, Germany). Anti-GFP immunohistochemistry was performed on brain sections using Vector ABC kit with ImmPact DAB substrate (Vector Laboratories, Burlingame, Calif.) based on suppliers instructions. Samples were imaged using a Nikon Eclipse TE2000-E fluorescence microscope equipped with a Ds-Fi1 camera (Nikon Inc., Melville, N.Y.). Average CSMN diameter was measured using Elements Software (Nikon Inc., Melville, N.Y.). Numbers of healthy CSMN apical dendrites and apical dendrites containing large vacuoles were quantified and presented as percentage of healthy (or is it vacuolated) dendrites. Statistical analyses were based on the average numbers for each mouse, and not based on total individual number of counts. All statistical analyses were performed using Prism software (Graphpad Software Inc., La Jolla, Calif.). Statistically significant differences were determined after either one-way ANOVA with post hoc Tukey's multiple comparison tests or t-test. Statistically significant differences were considered at p<0.05, and values were expressed as the mean±SEM.

### Example 2—UCHL1 is Necessary and Sufficient for Maintaining Cytoarchitectural Integrity of Upper Motor Neurons

Abstract

UCHL1 (ubiquitin C-terminal hydrolase-L1) is crucial for maintaining free ubiquitin levels especially in neurons. In Uchl1^{nm3419} (UCHL1^{-/-}) mice, which lack all UCHL1 function, the corticospinal motor neurons (CSMN, the upper motor neurons in mice) show early, selective, progressive and profound degeneration, which become evident by massive spine loss and apical dendrite disintegration. Upper motor neuron (UMN) degeneration is the defining characteristics of motor neuron disorders that affect voluntary movement, such as amyotrophic lateral sclerosis, primary lateral sclerosis, and hereditary spastic paraplegia. Thus, it is important to determine whether UCHL1 function would be sufficient to improve UMN health and integrity. Eliminating UCHL1 only from layer 5 of subcerebral projection neurons was sufficient to generate neuronal defects similar to that observed in the UCHL1^{-/-} mice, and when UCHL1 activity was ablated from spinal motor neurons (SMN), the corticospinal motor neuron (CSMN) remained intact. In addition, using AAV mediated gene delivery, we demonstrated that restoring UCHL1 expression specifically in CSMN of UCHL1^{-/-} mice, successfully improved the integrity of spines and apical dendrites. Our results bring a novel mechanistic insight on the importance of UCHL1 for the health and integrity of upper motor neurons, which are critically important for the initiation and modulation of voluntary movement in patients.

Introduction

Motor neuron circuitry is one of the most complex circuitries in our body; it has important cellular and neuronal components both in the motor cortex and in the spinal cord and is responsible for the initiation and modulation of voluntary movement.¹⁻³ The proper function of the motor neuron circuitry is critical for the reflection of our cognition to our actions. Thus, our ability to perform specific tasks with very high precision distinguishes us from other mammals and help define us as “humans”.

In motor neuron diseases, this complex circuitry degenerates leading to paralysis of the patient and eventual death. There has been a long debate in the field about defining the important components of the circuitry so that targeted therapies can be developed. Many argued that the corticospinal motor neuron (CSMN) or the upper motor neuron (UMN) death is mediated by a “die back” mechanism, which speculates that the UMN degeneration is a byproduct or a consequence of ongoing degeneration that moves towards the cortex, rendering UMN death as a byproduct of the ongoing degeneration.⁴ This hypothesis unfortunately eliminates their importance and relevance and diminishes the enthusiasm of targeting UMNs as a cellular target for improved motor function in patients.⁵

Recent building evidence, however, began to reveal that the cortical component of the motor neuron circuitry degenerates early in diseases, it is not dependent on SMN death, and the cortex is indeed a proper target for therapeutic interventions.^{1, 6-11} Furthermore, suppression of the mutant SOD1 only in the motor cortex was enough to delay disease onset in SOD1^{G93A} rat model of ALS and extend their survival.¹² Recently, transplantation of neural progenitor cells expressing glial-cell-line-derived neurotrophic factor (GDNF) into the motor cortex in SOD1^{G93A} rat model of ALS provided neuroprotection in both motor cortex and spinal cord, as well as delaying the disease pathology and extending the life span of the animals.¹³ These findings now reveal the importance of CSMN and UMN to disease pathology, and suggest their cellular modulation as a treatment strategy.^{14,15}

CSMN reside in layer 5 of the motor cortex and their apical dendrite extend towards upper layers, branching and arborizing especially within layer 2/3.² Their axons pass through the striatum, pons and entering spinal cord via primary decussation, and innervating appropriate targets. We consider them the “spokesperson” of the cerebral cortex for the initiation and modulation of voluntary movement and their ability to receive, integrate, translate and transmit cerebral cortex's input towards spinal cord targets. CSMN vulnerability and degeneration is well documented in motor neuron diseases (MND)¹⁶ such as hereditary spastic paraplegia (HSP), primary lateral sclerosis (PLS), and their death has been mostly considered to be secondary to sensory motor neuron (SMN) death in amyotrophic lateral sclerosis (ALS).^{4, 5, 17}

Cortical hyperexcitability has been shown to be an early event and an important mechanism in MND.^{8, 9, 18} Dendritic spine loss and vacuolation in CSMN preceded their degeneration in hSOD1^{G93A} ALS mouse model.¹⁹ Moreover, spine loss in motor cortex layer 5 pyramidal neurons was shown to be a very early event in pre-symptomatic hSOD1^{G93A} mice.²⁰⁻²²

UCHL1 is an important component of the ubiquitin-proteasome system (UPS) and can either add to or remove ubiquitin from polyubiquitin chains.²³⁻²⁵ Mutations in UCHL1 gene cause autosomal recessive spastic paraplegia-79 (SPG79) (MIM Number: #615491).²⁶⁻²⁹ Patients with UCHL1 mutations developed spasticity, with involvement of the upper motor neurons.²⁶ We have recently shown that in the absence of UCHL1 function, CSMN undergo massive cellular degeneration.³⁰ The UCHL1^{-/-} mice, which lack all UCHL1 function,³¹ display motor function defects and progressive CSMN loss that accompanied by vacuolated apical dendrites, spine loss, and increased ER stress.³⁰ Interestingly, the SMN in mice are also affected, which results in disintegration of neuromuscular junctions (NMJ) and loss of function without an avert cell loss.³² Therefore, the question of relative contribution of CSMN and SMN to MND phenotype cannot be answered since both cortical and spinal components are affected in the absence of UCHL1 function.

In an effort to bring a mechanistic insight for the UMN degeneration in the absence of UCHL1 function, and to investigate whether introduction of UCHL1 only in the CSMN would be sufficient to improve their integrity, we knocked out UCHL1 protein selectively in the large SCPN in layer 5 or in SMN in the spinal cord by mating floxed UCHL1 (UCHL1^{f/f}) mice with Rbp4 (Rbp4^{cre} UCHL1^{f/f}) or HB9 (HB9^{cre} UCHL1^{f/f}) cre mice. AAV2-GFP mediated retrograde labeling of CSMN^{2, 19} allowed detailed visualization and assessment of CSMN health and integrity, especially at the site of the apical dendrite. Conditional knockout of UCHL1 only in SCPN was sufficient to replicate the CSMN pathology observed in full mutant mice. Targeted AAV2-mediated delivery of the UCHL1 only to CSMN was sufficient to improve their cytoarchitectural integrity. Our findings reveal the importance of UCHL1 function for improving CSMN health in a cell-autonomous manner, and direct delivery of UCHL1 is sufficient to improve spine loss and apical dendrite stability, two important cytoarchitectural defects observed in a broad spectrum of ALS patients.

Results

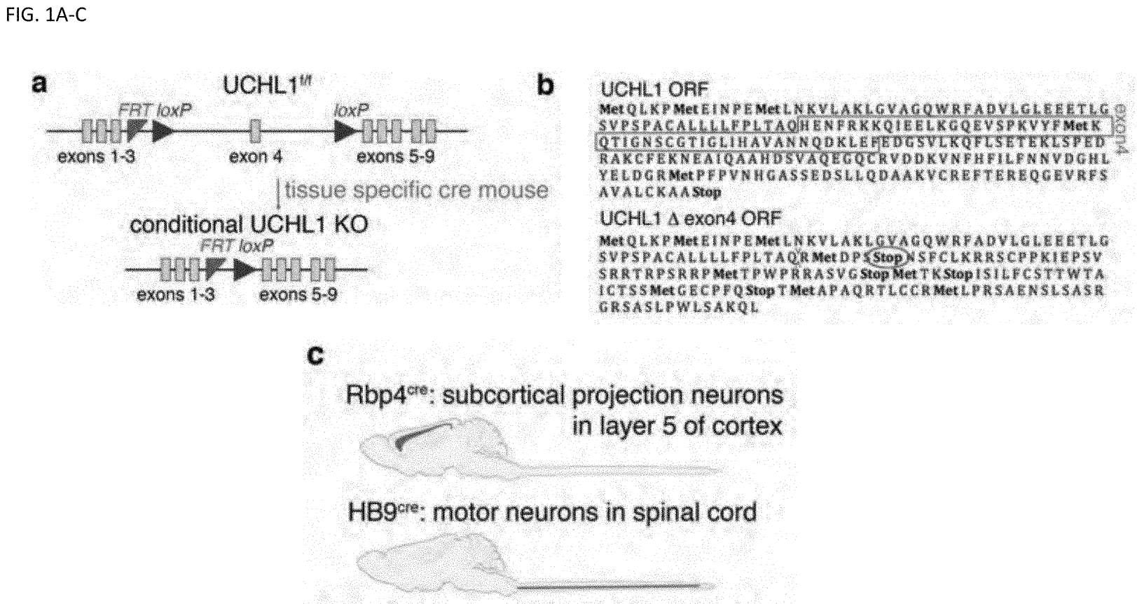

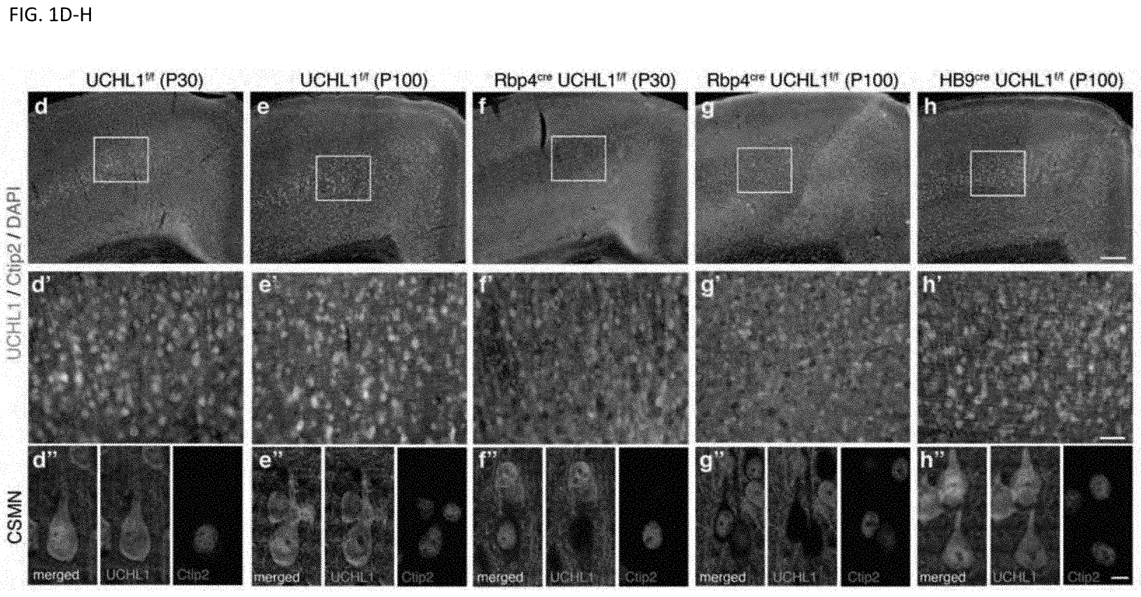

Site-specific in vivo deletion of UCHL1. We previously reported that in the absence of UCHL1 function, unlike other cortical neurons, CSMN undergo profound degeneration with spine loss and disintegration of apical dendrite.³⁰ To investigate the potential impact of the SMN dysfunction on the observed cellular defects on CSMN, and to assess the cell-type specific importance of UCHL1 function for the upper motor neurons, we generated conditional mutant mice that lacked UCHL1 function specifically in the brain and the spinal cord. First the floxed UCHL1 mice (UCHL1^{f/f}), in which the exon 4 of the UCHL1 gene is flanked by loxP sites, were generated (FIG. 1A). In the presence of cre recombinase, two loxP sites in introns 3 and 4 are recombined leading to deletion of UCHL1 exon 4. This deletion introduces a de novo stop codon shortly after exon 3 in the UCHL1 open reading frame (FIG. 1B), eliminating the production of a functional UCHL1 protein. Therefore, when UCHL1^{f/f} mice were crossed with Rbp4^{cre} mice, in which cre recombinase is expressed under the control of the Rbp4 promoter targeting SCPN in layer 5, including the CSMN in the primary motor cortex,³³⁻³⁶ the UCHL1 is deleted from the CSMN of the Rbp4^{cre} UCHL1^{f/f} conditional mutant mice (FIG. 1C). “Die-back” hypothesis^{4, 5} suggests that the pathology observed in CSMN of the UCHL1^{-/-} mice³⁰ could be attributed to defects in SMN.³² Therefore UCHL1^{f/f} mice were crossed with HB9^{cre} mice³⁷⁻⁴² to generate the HB9^{cre} UCHL1^{f/f} mice, which lack UCHL1 protein specifically in SMN (FIG. 1C), and to determine whether depletion of UCHL1 function in the SMN would have an impact on the health of CSMN.

As expected, Ctip2⁺ CSMN that normally have high levels of UCHL1 protein were not affected in the UCHL1^{f/f} mice at P30 (earliest time point investigated in this study; FIGS. 1D and D′) and P100 (FIGS. 1E and E′), as CSMN continued to express endogenous UCHL1. However, even though UCHL1 expression was detected in other neurons within the motor column, CSMN completely lacked UCHL1 protein in the Rbp4^{cre} UCHL1^{f/f} mice both at P30 (FIGS. 1F and F′) and P100 (FIGS. 1G and G′). CSMN soma was devoid of any UCHL1 expression in these transgenic mice at these ages, revealing a robust and prolonged depletion of UCHL1 protein selectively in the CSMN of these mice in vivo (FIG. 1D″-G″). In striking contrast, CSMN of HB9^{cre} UCHL1^{f/f} mice continued to express endogenous levels of UCHL1 and their levels were comparable to that of UCHL1^{f/f} mice at P100 (FIG. 1H-H″).

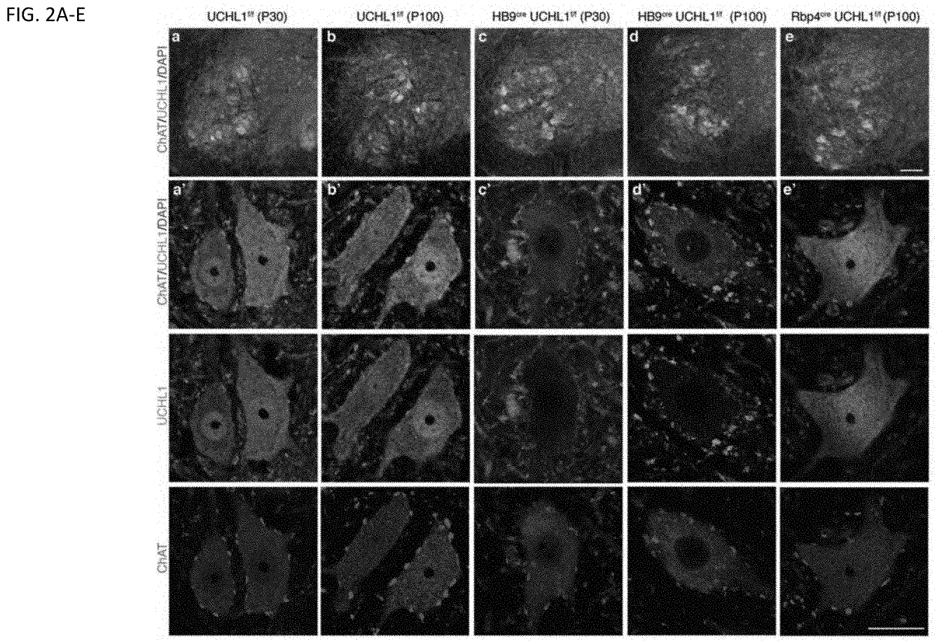

The expression of UCHL1 in the spinal cord, mainly in the SMN revealed that their expression profile was not affected in the UCHL1^{f/f} mice at P30 (FIGS. 2A and A′) or P100 (FIGS. 2B and B′). In contrast, SMN of HB9^{cre} UCHL1^{f/f} mice lacked UCHL1 expression at both P30 (FIGS. 2C and C′) and P100 (FIGS. 2D and D′). Especially the large alpha motor neurons were devoid of UCHL1 expression in the ventral horn of the spinal cord (FIG. 2C′-D′), revealing a long-term and stable depletion of UCHL1 in the neurons that are primarily affected in MND. However, the SMN of the Rbp4^{cre} UCHL1^{f/f} mice continued to express endogenous levels UCHL1 even at P100 (FIGS. 2E and E′), further confirming selective and robust deletion of UCHL1 in CSMN of the Rbp4^{cre} UCHL1^{f/f} mice and in the SMN of HB9^{cre} UCHL1^{f/f} mice. It is also important to note that the absence of UCHL1 function did not affect the birth and specification of neither CSMN nor the SMN, as Ctip2⁺ CSMN and ChAT⁺ SMN were detected in both of these conditional mutant lines.

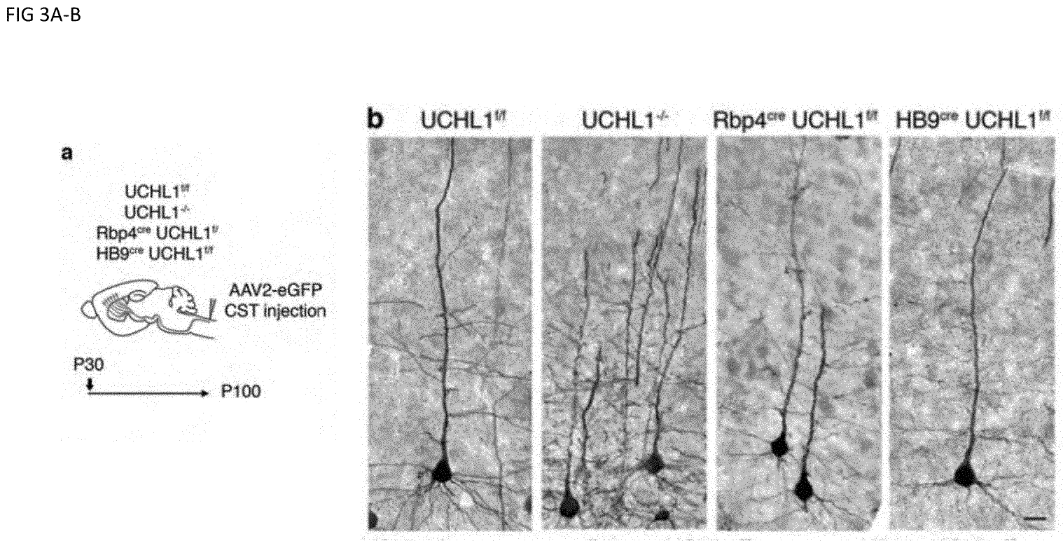

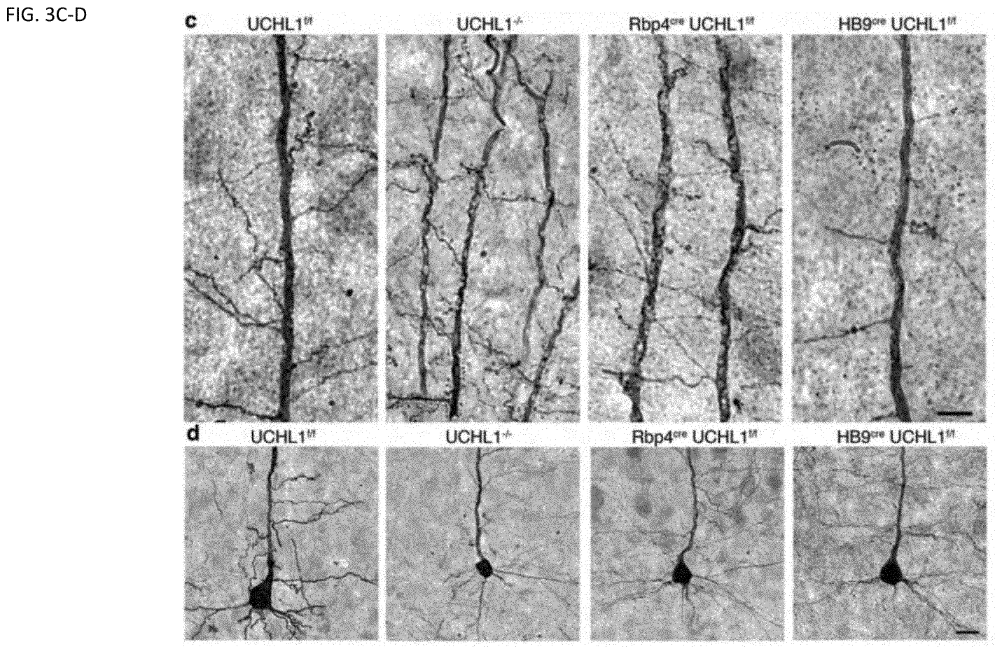

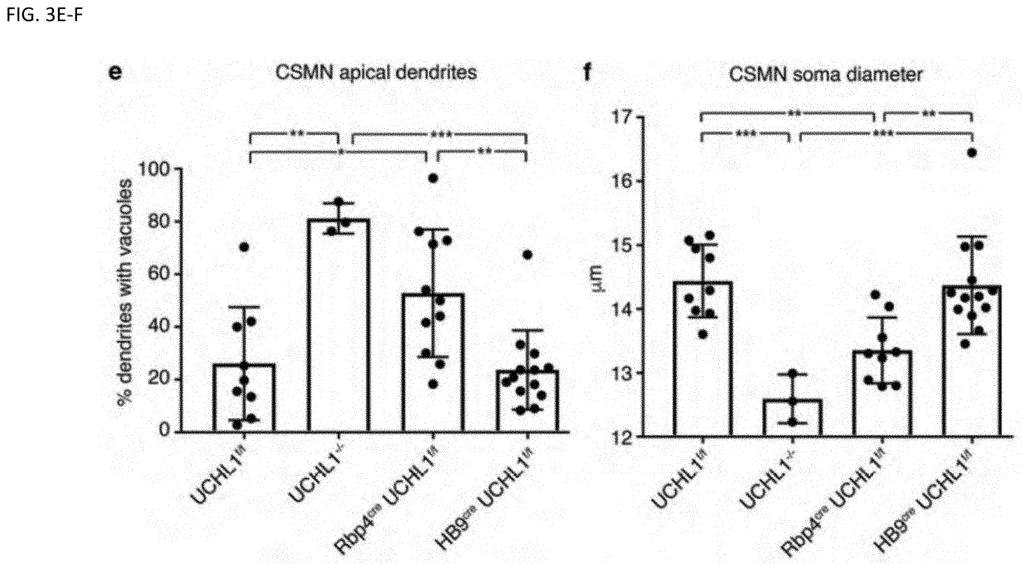

CSMN defects in the absence of UCHL1 function are cell autonomous. To investigate how site-directed deletion of UCHL1 function specifically from upper and lower motor neurons affects the cytoarchitectural integrity of CSMN, they were retrogradely transduced by AAV-2 eGFP, upon direct injection into the corticospinal tract at P30^{2, 19, 30} and GFP⁺ CSMN were analyzed at P100 (FIG. 3A). CSMN of UCHL1^{f/f} control mice demonstrated healthy cellular morphology, with large pyramidal cell bodies, prominent apical dendrites, and spines throughout the dendrites, mainly in layers 2/3 (FIG. 3B-D). The soma size of CSMN (FIG. 3F) in UCHL1^{-/-} (12.59±0.22 μm, n=219 CSMN; n=9 mice) and Rbp4^{cre} UCHL1^{f/f} mice (13.35±0.17 μm, n=469 CSMN; n=9 mice) were both significantly reduced when compared to the CSMN of UCHL1^{f/f} mice (14.44±0.19 μm, n=457 CSMN; n=9 mice; UCHL1^{f/f} vs. UCHL1^{-/-} p=0.0007; UCHL1^{f/f} vs. Rbp4^{cre} UCHL1^{f/f} p=0.0052), but they were comparable to each other (p=0.2941). Interestingly, the soma size of CSMN in HB9^{cre} UCHL1^{f/f} mice (14.37±0.21 μm, n=440 CSMN; n=13 mice) was comparable to that of CSMN in UCHL1^{f/f} (p=0.9944), suggesting that deletion of UCHL1 in the SMN did not have profound impact on the overall health of CSMN.

Since one of the most striking cellular pathology observed in diseased CSMN and CSMN that lack UCHL1 function was vacuolization and profound disintegration of apical dendrites,³⁰ we next investigated whether the integrity of apical dendrites was equally compromised in Rbp4^{cre} UCHL11^{f/f} and HB9^{cre} UCHL1^{f/f} mice. In line with previous reports, UCHL1^{-/-} mice (81.16±3.31%, n=357 apical dendrites, n=3 mice) exhibited significantly higher percentage of vacuolated CSMN apical dendrites (FIG. 3E) than CSMN of UCHL1^{f/f} mice (26.08±7.16%, n=654 apical dendrites, n=9 mice, p=0.0011). Different from UCHL1^{f/f} mice, majority of CSMN in Rbp4^{cre} UCHL1^{f/f} mice also had vacuolated apical dendrites (52.82±7.29%, n=701 apical dendrites, n=11 mice; p=0.0235). Interestingly, CSMN of UCHL1^{-/-} and Rbp4^{cre} UCHL1^{f/f} mice were comparable (p=0.1401). In contrast, CSMN of HB9^{cre} UCHL1^{ff} mice, which lacked UCHL1 only in the spinal cord, leaving UCHL1 intact in the brain, displayed healthier morphology and percent apical dendrites with vacuoles were comparable to the CSMN of UCHL1^{f/f} mice (23.68±8.68%, n=842 apical dendrites, n=13 mice; p=0.992).

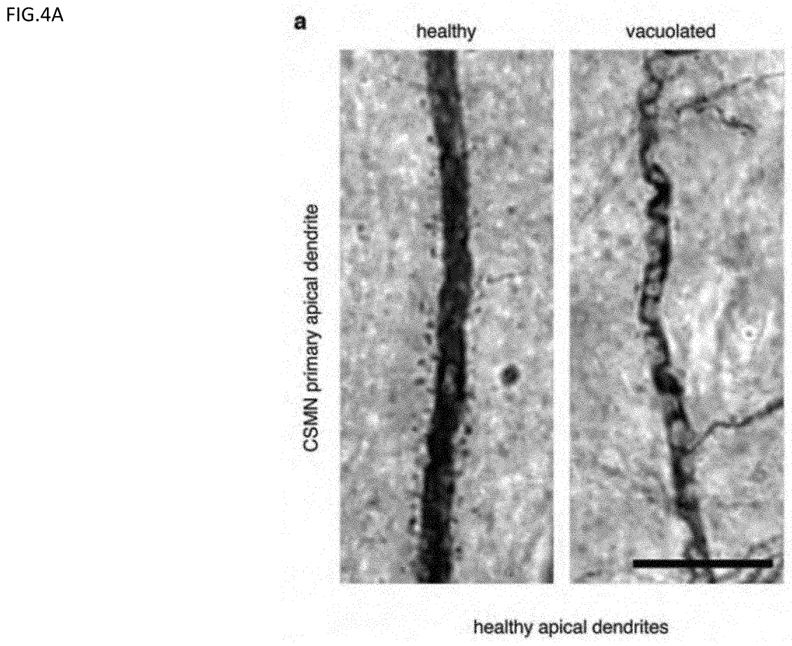

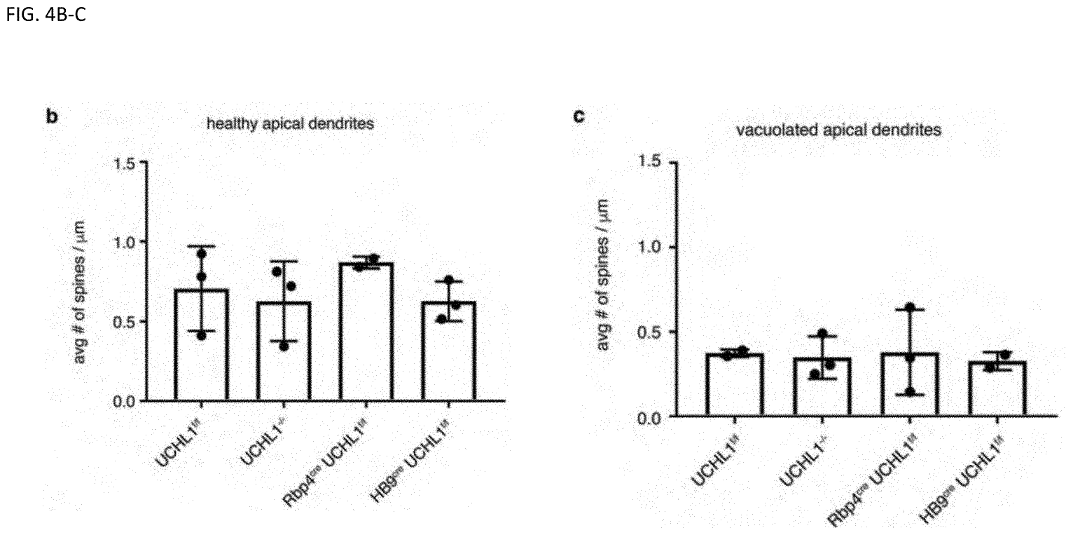

Since we previously detected spine loss in Betz cells of a broad spectrum of ALS patients,⁴³ and in the CSMN of UCHL1^{-/-} mice,³⁰ we next investigated a potential correlation between apical dendrite disintegration and spine loss (FIG. 4). Even though higher percentages of CSMN displayed vacuolization and disintegration in the UCHL1^{-/-} and HB9^{cre} UCHL1^{f/f} mice, similar correlation was observed with respect to vacuolization and spine loss regardless of the genotype. Average number of spines per μm of healthy primary apical dendrite of the CSMN in UCHL1^{f/f} (0.70±0.15 spines/μm, n=3 mice), UCHL1^{-/-} (0.63±0.14 spines/μm, n=3 mice), Rbp4^{cre} UCHL1^{f/f} (0.87±0.02 spines/μm, n=3 mice), and HB9^{cre} UCHL1^{f/f} mice (0.63±0.07 spines/μm, n=3 mice) were comparable at P100 (FIG. 4B). Similarly, the average number of spines per μm of the vacuolated and disintegrating primary apical dendrite of the CSMN in UCHL1^{f/f} (0.37±0.01 spines/μm, n=3 mice), UCHL1^{-/-} (0.35±0.07 spines/μm, n=3 mice), Rbp4^{cre} UCHL1^{f/f} (0.38±0.15 spines/μm, n=3 mice) and HB9^{cre} UCHL1^{f/f} mice (0.33±0.03 spines/μm, n=3 mice) were similar (FIG. 4C). However, diseased apical dendrites always had reduced number of spines than healthy apical dendrites in each genotype, further supporting a strong correlation between presence of spines and the integrity of the apical dendrite.

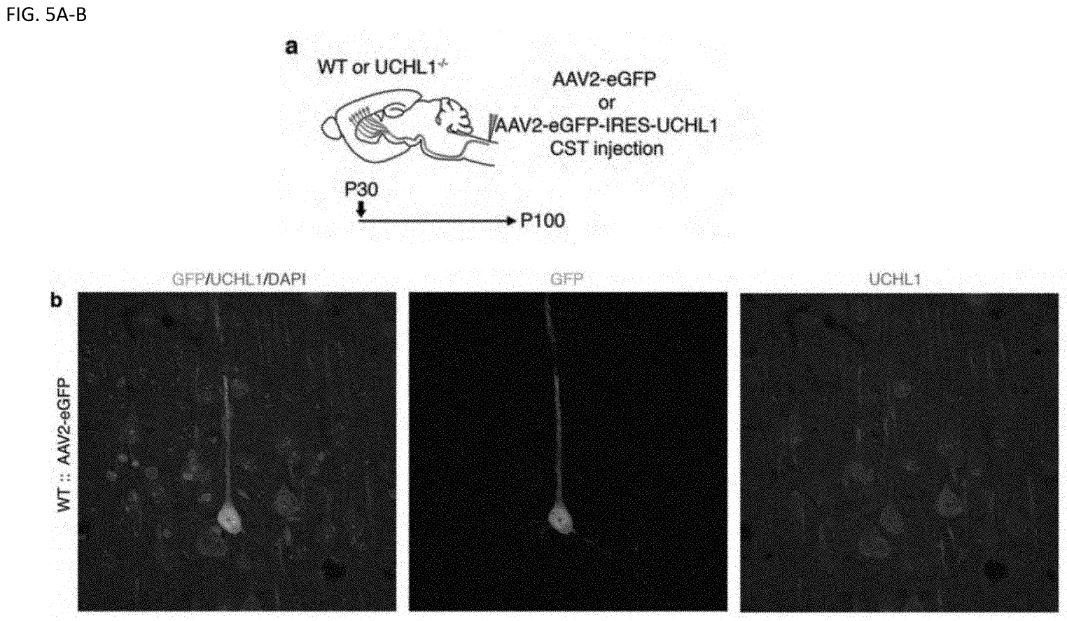

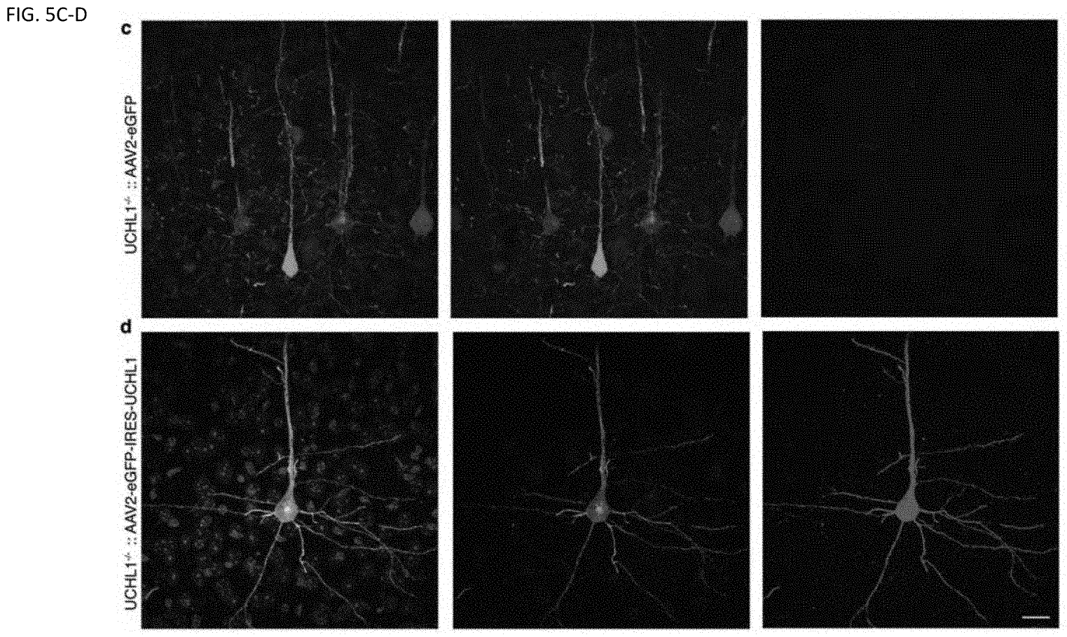

Directed UCHL1 delivery to CSMN is sufficient to improve CSMN integrity. In a separate set of experiments, we took advantage of AAV2 mediated gene delivery to restore UCHL1 function only in the CSMN of the UCHL1^{-/-} mice. AAV2 containing an eGFP-IRES-UCHL1 bicistronic expression vector was injected into the corticospinal tract (CST) that lies within the dorsal funiculus of the UCHL1^{-/-} mice at P30 and CSMN were analyzed at P100 (FIG. 5A). Wild type (WT) and UCHL1^{-/-} mice that underwent same retrograde labeling surgery using the AAV2-GFP vector alone that does not contain the UCHL1 coding sequence were used as positive and negative controls, respectively. WT CSMN that is retrogradely labeled by AAV2-eGFP express endogenous UCHL1 in the WT mice (FIG. 5B). As expected, UCHL1 protein is not detected in UCHL1^{-/-} mice, and CSMN that are retrogradely labeled with AAV2-eGFP also lacks UCHL1 expression (FIG. 5C). In contrast, when CSMN are retrogradely transduced with AAV2-eGFP-IRES-UCHL1, which leads to the expression of both GFP and UCHL1 proteins (not a fusion protein due to the bicistronic expression vector containing the IRES between two coding sequences), high levels of UCHL1 expression is detected only in GFP⁺ transduced CSMN (FIG. 5D). These experiments confirm effective transduction of CSMN and directed gene delivery only to CSMN in the motor cortex.

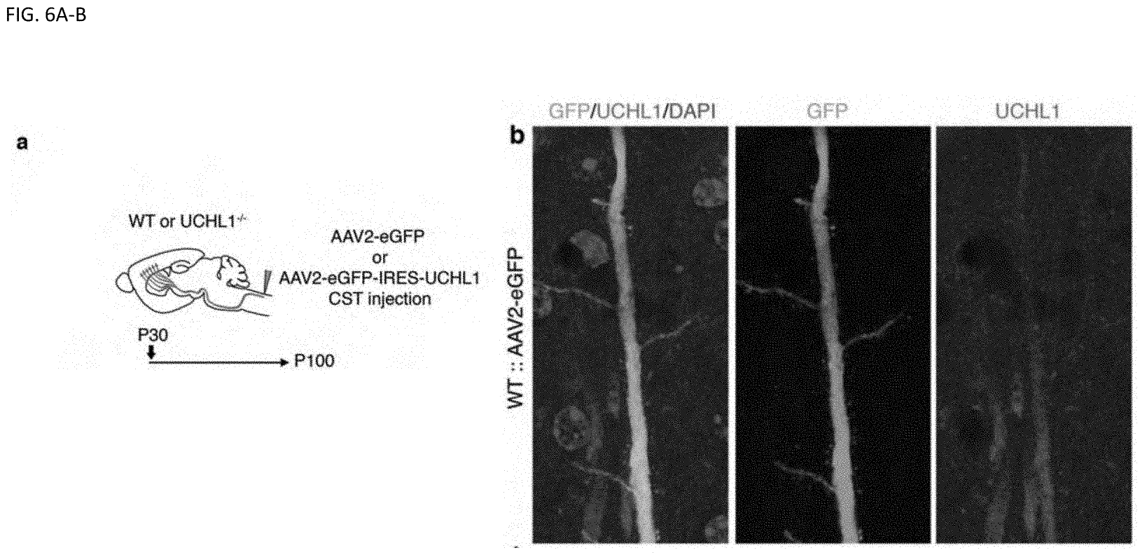

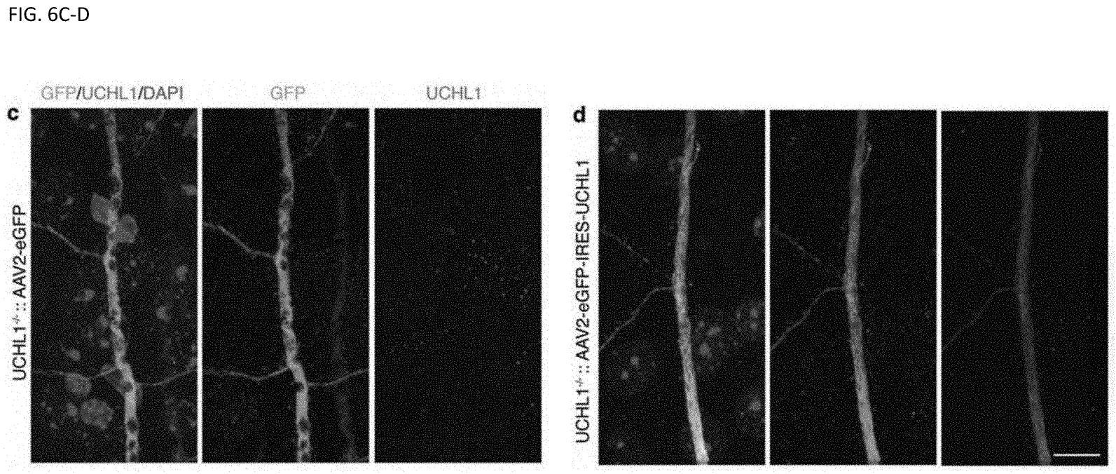

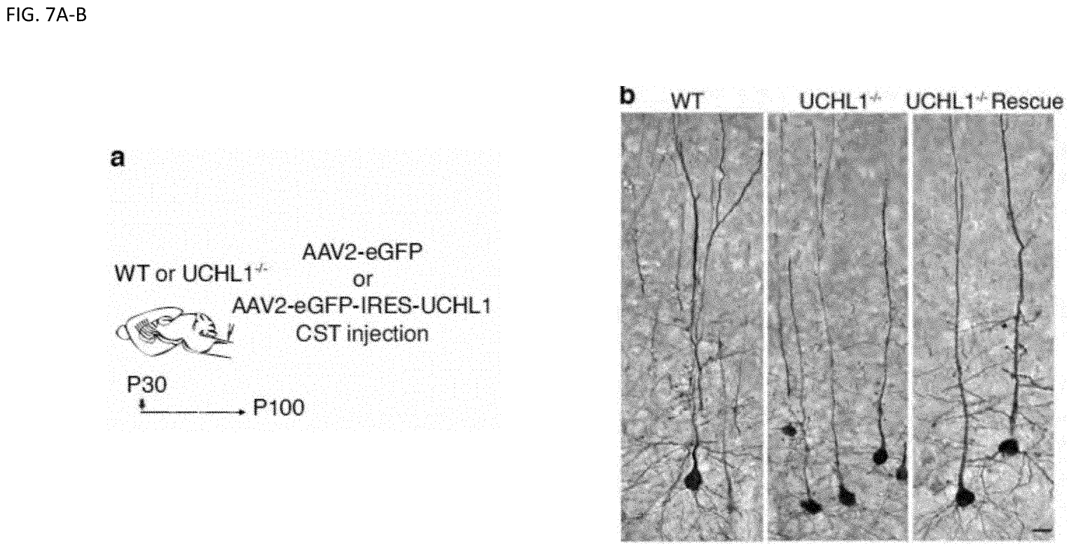

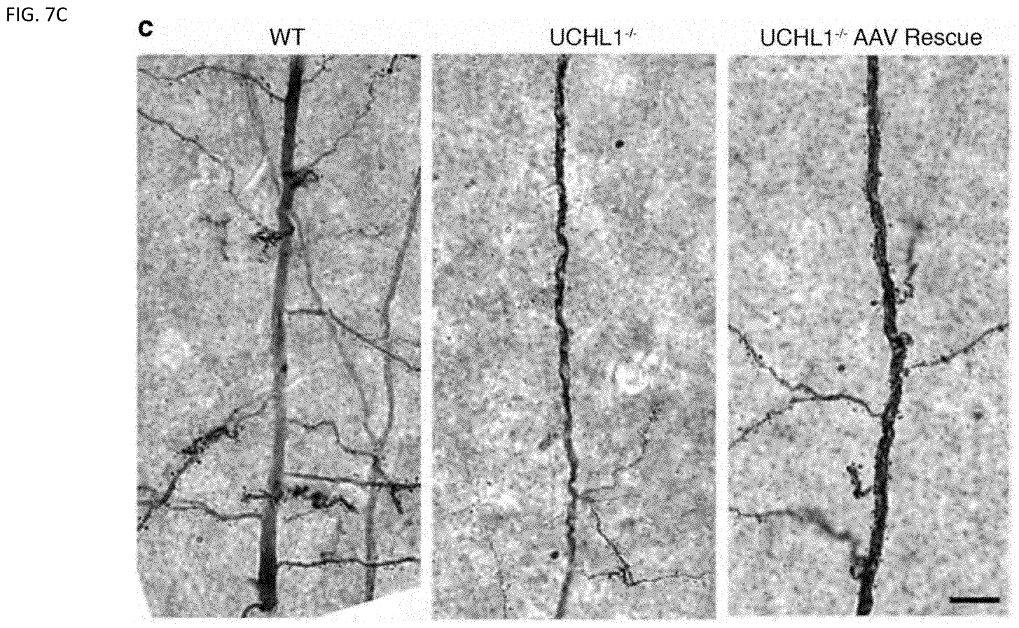

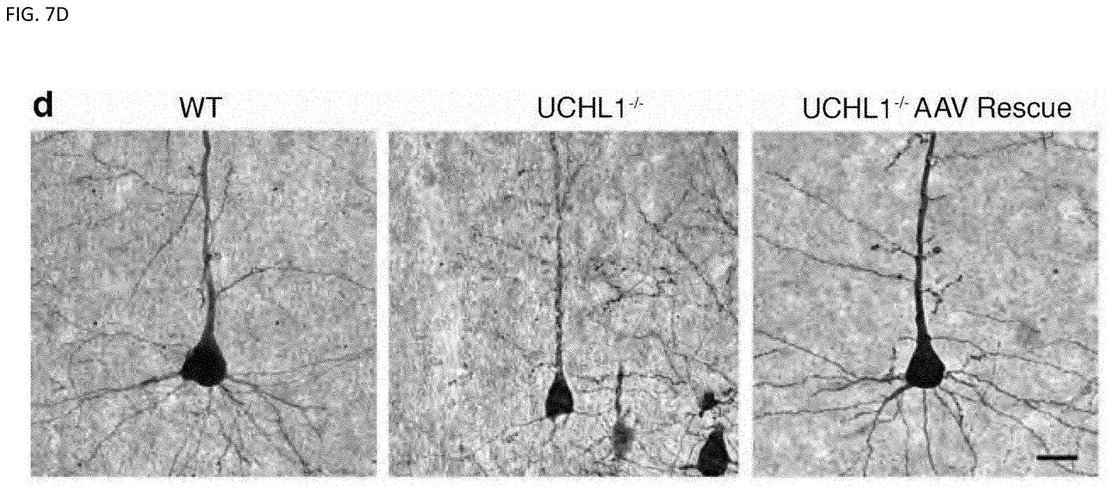

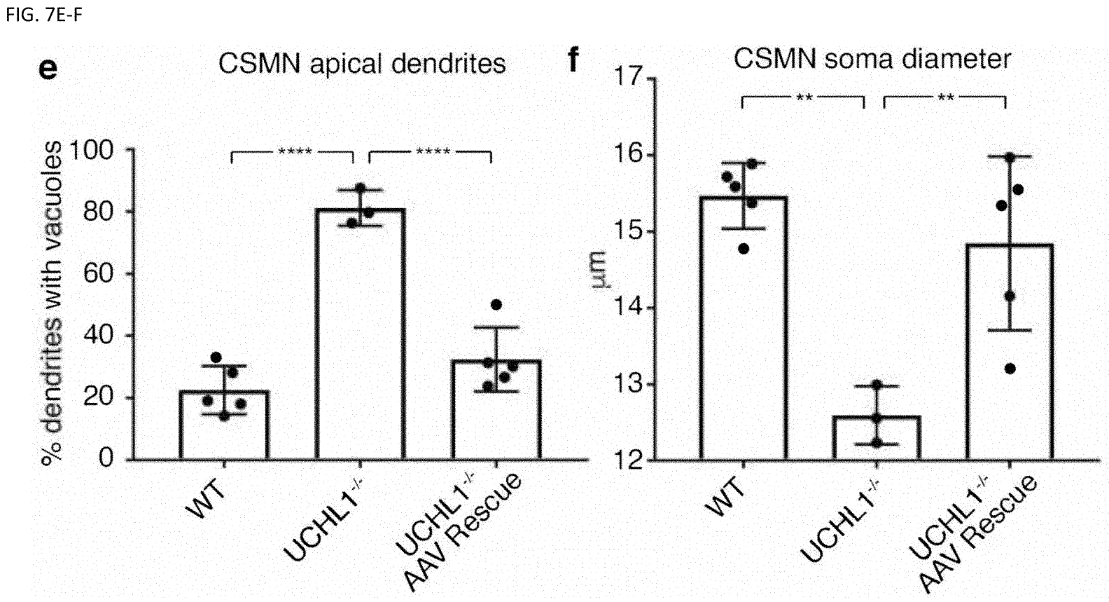

AAV2-eGFP-IRES-UCHL1 induces both UCHL1 and eGFP expression simultaneously, and because eGFP expression can be detected throughout the neuron, the integrity of the apical dendrites can also be assessed with precision (FIG. 6). In order to investigate whether UCHL1 delivery improves the integrity of CSMN, we performed eGFP immunohistochemistry to better visualize CSMN. Apical dendrites of WT mice were intact (FIG. 6B), but the apical dendrites of UCHL1^{-/-} mice were mostly filled with vacuoles that are in different shapes and sizes (FIG. 6C). Most interestingly, upon direct delivery of UCHL1, specifically to the CSMN of UCHL1^{-/-} mice, the integrity of the apical dendrites was dramatically improved; vacuolization of the apical dendrites was reduced and at times completely eliminated (FIG. 6D). We then performed quantitative assessment to investigate the extent of UCHL1-mediated rescue (FIG. 7).

CSMN of WT mice displayed healthy robust apical dendrites (22.57±3.48%, n=735 apical dendrites, n=5 mice), whereas CSMN of UCHL1^{-/-} mice contained significantly higher percentages of vacuolated dendrites (81.16±3.31%, n=357 apical dendrites, n=3 mice; p<0.0001; FIGS. 7C and E). When CSMN in UCHL1^{-/-} mice were rescued by AAV2-eGFP-IRES-UCHL1 transduction, the integrity of apical dendrites was improved and the percent apical dendrites with vacuoles were dramatically reduced (32.41±4.59%, n=195 apical dendrites, n=5 mice). They became similar and comparable to that of WT mice (p=0.2125) and significantly lower than that of UCHL1^{-/-} that are transfected by AAV2-eGFP alone (p<0.0001).

In addition, while the soma diameter of CSMN in UCHL1^{-/-} mice were significantly smaller (12.59±0.22 μm, n=457 cell bodies, n=9 mice) than that of WT mice (15.47±0.19 μm, n=986 cell bodies, n=5 mice; p=0.0014; FIGS. 7 D and F), when UCHL1 expression was introduced to the UCHL1^{-/-} mice, soma diameter was also restored (14.85±0.50 μm, n=137 cell bodies, n=5 mice), approaching to the size of CSMN in WT mice (p=0.452). These results, further confirm that introduction of UCHL1 only to CSMN is sufficient to improve its health and cytoarchitectural integrity, even when SMN lack UCHL1 activity.

Discussion

Our results show proof for the cell-autonomous mechanisms responsible for UMN degeneration. Their demise is not directly correlated to or a byproduct of SMN health. When UCHL1 function is selectively deleted only from SMN, this does not translate to neuronal degeneration or vulnerability of CSMN. Likewise, CSMN continue to undergo degeneration when UCHL1 function is selectively ablated from CSMN, but left intact in SMN. In addition, directed gene delivery of UCHL1 only to CSMN in the UCHL1^{-/-} mice is sufficient to improve neuronal cytoarchitectural integrity of CSMN, even when the entire nervous system including SMN continue to lack all UCHL1 function. The neuronal degeneration observed in the cortical and the spinal components of the circuitry do not appear to be consequential.

The UCHL1^{-/-} mice and the transgenic mice generated to deplete UCHL1 function selectively from layer 5 pyramidal neurons in the cortex and the SMN of the spinal cord, offered a unique tool to investigate the viability of the “die-back” hypothesis, which unfortunately diminished the importance of upper motor neuron degeneration as one of the causes of neuropathology and eliminated their chance to be considered as a potential cellular target for therapeutic interventions. Here, we show that even when SMN lack UCHL1 function, direct delivery of UCHL1 only to CSMN in UCHL1^{-/-} mice, results in almost complete rescue of their neuronal stability, integrity of apical dendrites and spine. Therefore, CSMN health or degeneration is not a function of SMN. Direct cellular therapies to upper motor neurons would thus improve their health, stability and connectivity, regardless of SMN health.

UCHL1 is a unique protein. It has the ability to add and remove ubiquitin from proteins, playing an important role for their function, relocation within the cell, and ultimately determination of their fate for recycling.²³⁻²⁵ UCHL1 function is required to ensure free ubiquitin levels are stable in cells, especially in neurons. The proper modulation of the UPS requires functional UCHL1. Even though all neurons ubiquitously express UCHL1, the upper motor neurons have high levels of UCHL1 throughout life.

Interestingly, mutations in the UCHL1 gene, which is located on chromosome 4p13, resulted in numerous forms of neurodegeneration that affects movement. For example, autosomal recessive spastic paraplegia-79 (SPG79) is caused by homozygous or compound heterozygous mutation in the UCHL1 gene (MIM Number: #615491). UCHL1^{GLU7ALA} missense mutation identified in a Turkish family lies within the ubiquitin-binding domain of UCHL1 protein and leads to near complete loss of hydrolase function.²⁶ All three siblings homozygous for the mutation have spasticity with upper motor neuron dysfunction, accompanied by early onset blindness, cerebellar ataxia, nystagmus, and dorsal column dysfunction. Two other missense mutations in the UCHL1 gene were identified in a Norwegian family.²⁸ Three siblings with compound heterozygous mutations UCHL1^{ARG178GLN} and UCHL1^{ALA216ASP} developed spasticity and ataxia following child onset blindness. The UCHL1^{ALA216ASP} mutation was reported to be insoluble and nonfunctional, whereas the UCHL1^{ARG178GLN} mutation leads to a 4-fold increase in the hydrolytic activity of UCHL1 protein. Recently, a third family from India was reported with two siblings carrying a deleterious homozygous splice-site variant predicted to cause splicing aberrations.²⁷ Both siblings have spasticity. Clinical features of all 8 patients with mutations in their UCHL1 gene and from three unrelated families share early neurodegeneration with spasticity. These clinical findings suggest the importance of UCHL1 and that its mutations lead to neurodegeneration with indications of UMN involvement.

Similar to patients with mutations in their UCHL1 gene, mouse models of UCHL1, especially the Uchl1^{nm3419} (UCHL1^{-/-}) mice used in this study lack all UCHL1 function and display early onset neurodegeneration, accompanied with spasticity, muscular atrophy and profound upper motor neuron degeneration with disintegrating apical dendrites and spine loss.^{32, 44, 45} Therefore, we think UCHL1^{-/-} mice offers a unique opportunity to investigate upper motor neuron degeneration especially with respect to UPS dysfunction and spine loss.

Synaptic plasticity is closely associated with morphological changes in spines.⁴⁶ In hippocampal neurons, NMDA receptor activation leads to UCHL1 activity and an increase in levels of free monomeric ubiquitin.⁴⁷ On the other hand, inhibition of UCHL1 activity reduces levels of free ubiquitin, increases spine size, decreases spine density,⁴⁷ and reduces LTP and basal synaptic transmission.⁴⁸ In gad mice with a spontaneous deletion of UCHL1 exons 7 & 8, there is a reduction in hippocampal CA1 LTP, along with reduced memory in passive avoidance learning and exploratory behavior.⁴⁹ Exogenous UCHL1 can rescue β-amyloid-induced reduction in LTP.⁴⁸ Ubiquitin homeostasis is critical for synaptic development and function, and ubiquitin deficiency may contribute to synaptic dysfunction in diseases.⁵⁰ Our data also show requirement of UCHL1 for the maintenance of apical dendrites and spines of CSMN, as its presence correlates with improved cytoarchitectural stability.

The apical dendrite is a critical site for proper modulation of upper motor neurons, as most of their long-distance and local connections are mediated by the spines located in layer 2-3 of the motor cortex. Upper motor neurons require their apical dendrites and spines to collect and integrate input from different neuron populations that modulate their function. Therefore, the integrity and the stability of the apical dendrite is paramount for upper motor neuron modulation and its function.^{2, 51-53} Since vacuolated apical dendrites have reduced spine density, the cortical connectivity of CSMN with disintegrating apical dendrites would not be possible.

We recently discovered that diseased Betz cells in a wide spectrum of ALS patients, including sporadic ALS, familial ALS and ALS-Frontotemporal Dementia display massive apical dendrite degeneration.^{1, 43, 54} This cellular pathology observed in CSMN of UCHL1^{-/-} mice³⁰ as well as numerous mouse models of ALS, such as hSOD1^{G93A, 19, 55} TDP-43^{A315T, 56-59} hPFN1^{G118V, 60} and Alsin^{-/-} mice.⁶¹ Therefore, upper motor neurons in different species and that become diseased due to many unrelated causes converge onto the same cellular pathology: massive apical dendrite degeneration with vacuolation and spine loss. It is thus important to develop treatment strategies that improve the health and the integrity of apical dendrites and spines of the upper motor neurons. Our studies reveal the importance of UCHL1 for the health of CSMN.

UCHL1 protein has been previously considered as a therapeutic target for post-traumatic brain injury and hypoxic injury. UCHL1 fused to the protein transduction domain of HIV-transactivating transduction protein (TAT-UCHL1) can transduce neurons after intraperitoneal (i.p.) injection into mice.⁶² In a controlled cortical impact (CCI) injury model of post-traumatic brain injury (TBI), TAT-UCHL1 treatment improved function of the ubiquitin-proteasome pathway, decreased activation of autophagy after CCI, attenuated axonal injury and increased hippocampal neuronal survival after CCI.⁶² During hypoxic injury, whereas pharmacologic inhibition of UCHL1 function exacerbates neuronal death induced by hypoxia, TAT-UCHL1 treatment provides neuroprotection.⁶³ In a mouse model of AD, TAT-UCHL1 restored synaptic function in hippocampal slices after oligomeric Aβ treatment, and improved retention of contextual learning in APP/PS1 mice upon i.p. injection.⁴⁸ Moreover, Aβ induced impairment of neurotrophin-mediated retrograde signaling can be rescued by increasing cellular UCHL1 levels upon TAT-UCHL1 treatment.⁶⁴ These, together with UCHL1 rescue with AAV2-GFP-UCHL1 suggest that UCHL1 could indeed be a therapeutic agent against acute injury, hypoxia as well as neurodegeneration.

Recent studies reveal that defects occur early in the motor cortex of ALS patients^{18, 65-69} and in CSMN of ALS disease models.^{19-22, 53, 55, 57-59, 61, 70} Most importantly, studies with rat models revealed that improving the health of CSMN not only improves the health of SMN but also the neuromuscular junction, further proving that CSMN are indeed viable targets for maintaining the health, integrity and connectivity of the motor neuron circuitry.^{12, 13} Therefore, investigation of CSMN's requirements for survival and improved health is of great importance, is clinically relevant, and has the potential to exponentially improve the success rates of future clinical trials of motor neuron diseases in which upper motor neurons and the motor neuron circuitry are affected.

Taken together, our results reveal that UMN degeneration is not a function of SMN health, and that they respond to direct gene delivery approaches. UMNs, which play a pivotal role for the initiation and modulation of movement, and their degeneration is one of the early hallmarks of neurodegenerative diseases in which motor neuron circuitry is impaired, are indeed cellular targets for direct gene modulation. UCHL1 emerges as a potential candidate. Its expression only in CSMN is sufficient to improve stability and integrity of apical dendrites, an important site for upper motor neuron modulation and disintegration is a common pathology observed in the Betz cells of a broad spectrum of ALS patients.

Materials and Methods

Mice. All animal procedures were approved by Northwestern University Animal Care and Use Committee and conformed to the standards of the National Institutes of Health. Uchl1^{nm3419} (UCHL1^{-/-}) mice carry a spontaneous 795 base-pair intragenic deletion that results in the removal of 24 base-pairs of exon 6 and 771 base-pairs of intron 6.³⁰⁻³² Heterozygous mice (UCHL1^{+/-}) were viable, fertile, and bred to generate UCHL1 deficient (UCHL1^{-/-}) mice. Survival times and motor function defects were comparable between males and females with 100% penetrance. Uchl1^{tm1a(EUcoMM)Hmgu} knockout first allele targeted embryonic stem cells were purchased from The European Conditional Mouse Mutagenesis (EUCOMM) Program. Floxed UCHL1 mice, in which exon 4 of the UCHL1 gene is flanked by LoxP sites, were generated with the assistance of Northwestern University Transgenic and Targeted Mutagenesis Laboratory. The lacZ/neo cassette flanked by FRT sites in intron 3 was deleted by crossing germline transgenic mice with Ella FLPeR mice (provided by Northwestern University Transgenic and Targeted Mutagenesis Laboratory), to generate the UCHL1^{f/f} mice. Rbp4^{cre} mice on a mixed background [Tg(Rbp4-cre)KL100Gsat/Mmucd; MMRRC stock #031125-UCD] were purchased from Mutant Mouse Regional Resource Center (MMRRC) at UC Davis Mouse Biology Program.^{33-36, 71, 72} HB9^{cre} mice [B6.129S1-Mnx1^{tm4(cre)Tmj}/J; JAX stock #006600] were purchased from Jackson labs.^{37, 38} Both floxed UCHL1 and Rbp4^{cre} mouse lines were backcrossed to C57BL/6J background for at least 8 generations. Conditional mutant mice were generated by crossing floxed UCHL1 mice with Rbp4^{cre} or HB9^{cre} mice. Primers used to determine genotype of Uch11^{nm349} mice are UCHL1 forward: tggacggctgtgtgtgctaatg (SEQ ID NO:9), WT reverse: ctaagggaagggtcttgctcatc (SEQ ID NO:10), mutant (Mt) reverse: gtcatctacctgaagagagccaag (SEQ ID NO:11), yielding 668 bp WT and 334 bp Mt PCR products. Primers used to determine genotype of floxed UCHL1 mice are forward: tagtccaatccttgtaccagttgg (SEQ ID NO:12) and reverse: ccatggttctagatgctgttgaatgc (SEQ ID NO:13), yielding 428 bp WT and 540 bp floxed UCHL1 products. Primers used to determine genotype of cre mice are forward: gcattaccggtcgatgcaacgagtgat (SEQ ID NO:14) and reverse: gagtgaacgaacctggtcgaaatcagt (SEQ ID NO:15), yielding a 408 bp product.

Generation of adeno-associated virus (AAV). AAV vectors were generated by the University of Pennsylvania Vector Core facility by triple transfection of subconfluent HEK293 cells using three plasmids: an AAV trans-plasmid encoding AAV2 capsid, an adenovirus helper plasmid pΔF6, and an AAV cis shuttle plasmid expressing eGFP driven by a CMV promoter (pENN.AAV.CMV.PI.eGFP.WPRE.bGH). The culture medium was collected, concentrated by tangential flow filtration and purified by iodixanol gradient ultracentrifugation as previously described.⁷³ pGEM-T vector plasmid containing the mouse UCHL1 cDNA ORF clone was purchased from Sino Biological (cat: MG50690-G, Wayne, Pa.), and the UCHL1 CDS was subcloned into a AAV plasmid with CBA promoter,⁷⁴ to generate pAAV.CBA.UCHL1-IRES-eGFP.WPRE plasmid that was packaged into AAV2 virus particles by the University of Pennsylvania Vector Core facility as described above.

Retrograde labeling and transduction surgeries. Surgeries were performed on a stereotaxic platform. Micro-injections were performed using pulled-beveled glass micro-pipettes attached to a nanojector (Drummond Scientific, Broomall, Pa.). CSMN were retrogradely labeled by AAV encoding eGFP (AAV2-eGFP; 621 nl containing 1.16×109 viral particles), injected into the CST at P30 and CSMN were retrogradely transduced as described.^{19, 30}

Tissue collection and histology. Mice were deeply anesthetized with ketamine (90 mg/kg) and xylazine (10 mg/kg) and perfused with 4% PFA in PBS. The brain was removed intact from each mouse, post-fixed by 4% PFA overnight and kept in PBS-sodium azide (0.01%) at 4° C. Brains were sectioned (coronal; 50 m) using a vibrating microtome (VT1000SLeica Instruments, Nussloch, Germany).

Immunocytochemistry. Immunocytochemistry was performed on every 6^{th} coronal section of mouse brains. Antigen retrieval was performed for Ctip2 immunocytochemistry; sections were treated with 0.01 M sodium citrate, pH 9.0, at 80° C. water bath for 2 hr prior to incubation with primary antibody. Primary antibodies were: anti-Ctip2 (1:500; Abcam, Cambridge, Mass.); anti-GFP (1:1000; Invitrogen, Grand Island, N.Y.), anti-ChAT (1:200; Millipore, Burlington, Mass.), and anti-UCHL1 (1:1000; ProteinTech, Rosemont, Ill.). After PBS washes, either fluorescent conjugated (AlexaFluor, Invitrogen, Grand Island, N.Y.) or biotinylated (Vector Laboratories, Burlingame, Calif.) secondary antibodies were used for detection.

Imaging, quantification, and statistical analysis. Nikon SMZ1500 and Nikon Eclipse TE2000-E fluorescence microscopes equipped with Intensilight C-HGFI (Nikon Inc., Melville, N.Y.) were used. Epifluorescence images were acquired using a Digital Sight DS-Qi1MC CCD camera (Nikon Inc., Melville, N.Y.) and light images were acquired using a Ds-Fi1 camera (Nikon Inc., Melville, N.Y.). Confocal images were collected using a Zeiss 510 Meta confocal microscope (Carl Zeiss Inc., Thornwood, N.Y.).

Numbers and diameters of CSMN were determined at P100. Average CSMN diameter (at least 100 neurons/mouse; n=3) was measured using Elements Software (Nikon Inc., Melville, N.Y.) OR Image J Software (NIH, Bethesda, Md.).

For CSMN spine density measurements, a measured segment of apical dendrites (in layer 2/3, in the primary apical dendrite) were selected and the total numbers of spines were counted in each segment (10 segments/mouse; n=3). The numbers were averaged and results were presented as average number of spines per μm, per genotype. Even though all statistical analyses were performed per genotype, the information of the total counts are included in the results section to be informative on the extent of the quantitative assessment.

Statistical analyses were based on the average numbers for each mouse, and not based on total individual number of counts. All statistical analyses were performed using Prism software (version 5.0a; Graphpad Software Inc., La Jolla, Calif.). Statistically significant differences were determined after either one-way ANOVA with post hoc Tukey's multiple comparison tests or t-test. Statistically significant differences were considered at p<0.05, and values were expressed as the mean±SEM.

### REFERENCES

1. Geevasinga, N, Menon, P, Ozdinler, P H, Kiernan, M C, and Vucic, S (2016). Pathophysiological and diagnostic implications of cortical dysfunction in ALS. Nature reviews Neurology 12: 651-661.
2. Jara, J H, Genc, B, Klessner, J L, and Ozdinler, P H (2014). Retrograde labeling, transduction, and genetic targeting allow cellular analysis of corticospinal motor neurons: implications in health and disease. Front Neuroanat 8: 16.
3. Lemon, R N (2008). Descending pathways in motor control. Annual review of neuroscience 31: 195-218.
4. Chou, S M, and Norris, F H (1993). Amyotrophic lateral sclerosis: lower motor neuron disease spreading to upper motor neurons. Muscle & nerve 16: 864-869.
5. Dadon-Nachum, M, Melamed, E, and Offen, D (2011). The “dying-back” phenomenon of motor neurons in ALS. J Mol Neurosci 43: 470-477.
6. Eisen, A, Kim, S, and Pant, B (1992). Amyotrophic lateral sclerosis (ALS): a phylogenetic disease of the corticomotoneuron? Muscle & nerve 15: 219-224.
7. Eisen, A, and Weber, M (2001). The motor cortex and amyotrophic lateral sclerosis. Muscle & nerve 24: 564-573.
8. Vucic, S, Cheah, B C, and Kiernan, M C (2009). Defining the mechanisms that underlie cortical hyperexcitability in amyotrophic lateral sclerosis. Experimental neurology 220: 177-182.
9. Vucic, S, Cheah, B C, Yiannikas, C, and Kiernan, M C (2011). Cortical excitability distinguishes ALS from mimic disorders. Clin Neurophysiol 122: 1860-1866.
10. Geevasinga, N, Howells, J, Menon, P, van den Bos, M, Shibuya, K, Matamala, J M, et al. (2019). Amyotrophic lateral sclerosis diagnostic index: Toward a personalized diagnosis of ALS. Neurology 92: e536-e547.
11. Lacomis, D, and Gooch, C (2019). Upper motor neuron assessment and early diagnosis in ALS: Getting it right the first time. Neurology 92: 255-256.
12. Thomsen, G M, Gowing, G, Latter, J, Chen, M, Vit, J P, Staggenborg, K, et al. (2014). Delayed disease onset and extended survival in the SOD1G93A rat model of amyotrophic lateral sclerosis after suppression of mutant SOD1 in the motor cortex. The Journal of neuroscience: the official journal of the Society for Neuroscience 34: 15587-15600.
13. Thomsen, G M, Avalos, P, Ma, A A, Alkaslasi, M, Cho, N, Wyss, L, et al. (2018). Transplantation of Neural Progenitor Cells Expressing Glial Cell Line-Derived Neurotrophic Factor into the Motor Cortex as a Strategy to Treat Amyotrophic Lateral Sclerosis. Stem Cells 36: 1122-1131.
14. Baker, M R (2014). ALS—dying forward, backward or outward?Nature reviews Neurology 10: 660.
15. Piotrkiewicz, M, and Hausmanowa-Petrusewicz, I (2013). Amyotrophic lateral sclerosis: a dying motor unit? Frontiers in aging neuroscience 5: 7.
16. Novarino, G, Fenstermaker, A G, Zaki, M S, Hofree, M, Silhavy, J L, Heiberg, A D, et al. (2014). Exome sequencing links corticospinal motor neuron disease to common neurodegenerative disorders. Science 343: 506-511.
17. Fischer, L R, Culver, D G, Tennant, P, Davis, A A, Wang, M, Castellano-Sanchez, A, et al. (2004). Amyotrophic lateral sclerosis is a distal axonopathy: evidence in mice and man. Experimental neurology 185: 232-240.
18. Geevasinga, N, Menon, P, Sue, C M, Kumar, K R, Ng, K, Yiannikas, C, et al. (2015). Cortical excitability changes distinguish the motor neuron disease phenotypes from hereditary spastic paraplegia. Eur J Neurol 22: 826-831, e857-828.
19. Jara, J H, Villa, S R, Khan, N A, Bohn, M C, and Ozdinler, P H (2012). AAV2 mediated retrograde transduction of corticospinal motor neurons reveals initial and selective apical dendrite degeneration in ALS. Neurobiology of disease 47: 174-183.
20. Fogarty, M J, Noakes, P G, and Bellingham, M C (2015). Motor cortex layer V pyramidal neurons exhibit dendritic regression, spine loss, and increased synaptic excitation in the presymptomatic hSOD1(G93A) mouse model of amyotrophic lateral sclerosis. The Journal of neuroscience: the official journal of the Society for Neuroscience 35: 643-647.
21. Fogarty, M J, Mu, E W, Noakes, P G, Lavidis, N A, and Bellingham, M C (2016). Marked changes in dendritic structure and spine density precede significant neuronal death in vulnerable cortical pyramidal neuron populations in the SOD1(G93A) mouse model of amyotrophic lateral sclerosis. Acta Neuropathol Commun 4: 77.
22. Fogarty, M J (2019). Amyotrophic lateral sclerosis as a synaptopathy. Neural Regen Res 14: 189-192.
23. Day, I N, and Thompson, R J (2010). UCHL1 (PGP 9.5): neuronal biomarker and ubiquitin system protein. Progress in neurobiology 90: 327-362.
24. Bishop, P, Rocca, D, and Henley, J M (2016). Ubiquitin C-terminal hydrolase L1 (UCH-L1): structure, distribution and roles in brain function and dysfunction. Biochem J 473: 2453-2462.
25. Liu, Y, Fallon, L, Lashuel, H A, Liu, Z, and Lansbury, P T, Jr. (2002). The UCH-L1 gene encodes two opposing enzymatic activities that affect alpha-synuclein degradation and Parkinson's disease susceptibility. Cell 111: 209-218.
26. Bilguvar, K, Tyagi, N K, Ozkara, C, Tuysuz, B, Bakircioglu, M, Choi, M, et al. (2013). Recessive loss of function of the neuronal ubiquitin hydrolase UCHL1 leads to early-onset progressive neurodegeneration. Proceedings of the National Academy of Sciences of the United States of America 110: 3489-3494.
27. Das Bhowmik, A, Patil, S J, Deshpande, D V, Bhat, V, and Dalal, A (2018). Novel splice-site variant of UCHL1 in an Indian family with autosomal recessive spastic paraplegia-79. J Hum Genet 63: 927-933.
28. Rydning, S L, Backe, P H, Sousa, M M L, Iqbal, Z, Oye, A M, Sheng, Y, et al. (2017). Novel UCHL1 mutations reveal new insights into ubiquitin processing. Human molecular genetics 26: 1217-1218.
29. Blackstone, C (2018). Converging cellular themes for the hereditary spastic paraplegias. Current opinion in neurobiology 51: 139-146.
30. Jara, J H, Genc, B, Cox, G A, Bohn, M C, Roos, R P, Macklis, J D, et al. (2015). Corticospinal Motor Neurons Are Susceptible to Increased E R Stress and Display Profound Degeneration in the Absence of UCHL1 Function. Cereb Cortex 25: 4259-4272.
31. Walters, B J, Campbell, S L, Chen, P C, Taylor, A P, Schroeder, D G, Dobrunz, L E, et al. (2008). Differential effects of Usp14 and Uch-L1 on the ubiquitin proteasome system and synaptic activity. Molecular and cellular neurosciences 39: 539-548.
32. Genc, B, Jara, J H, Schultz, M C, Manuel, M, Stanford, M J, Gautam, M, et al. (2016). Absence of UCHL 1 function leads to selective motor neuropathy. Ann Clin Transl Neurol 3: 331-345.
33. Gerfen, C R, Paletzki, R, and Heintz, N (2013). GENSAT BAC cre-recombinase driver lines to study the functional organization of cerebral cortical and basal ganglia circuits. Neuron 80: 1368-1383.
34. Kim, J, Hughes, E G, Shetty, A S, Arlotta, P, Goff, L A, Bergles, D E, et al. (2017). Changes in the Excitability of Neocortical Neurons in a Mouse Model of Amyotrophic Lateral Sclerosis Are Not Specific to Corticospinal Neurons and Are Modulated by Advancing Disease. The Journal of neuroscience: the official journal of the Society for Neuroscience 37: 9037-9053.
35. Leone, D P, Heavner, W E, Ferenczi, E A, Dobreva, G, Huguenard, J R, Grosschedl, R, et al. (2015). Satb2 Regulates the Differentiation of Both Callosal and Subcerebral Projection Neurons in the Developing Cerebral Cortex. Cereb Cortex 25: 3406-3419.
36. Woodworth, M B, Greig, L C, Liu, K X, Ippolito, G C, Tucker, H O, and Macklis, J D (2016). Ctip1 Regulates the Balance between Specification of Distinct Projection Neuron Subtypes in Deep Cortical Layers. Cell Rep 15: 999-1012.
37. Arber, S, Han, B, Mendelsohn, M, Smith, M, Jessell, T M, and Sockanathan, S (1999). Requirement for the homeobox gene Hb9 in the consolidation of motor neuron identity. Neuron 23: 659-674.
38. Yang, X, Arber, S, William, C, Li, L, Tanabe, Y, Jessell, T M, et al. (2001). Patterning of muscle acetylcholine receptor gene expression in the absence of motor innervation. Neuron 30: 399-410.
39. Wichterle, H, Lieberam, I, Porter, J A, and Jessell, T M (2002). Directed differentiation of embryonic stem cells into motor neurons. Cell 110: 385-397.
40. Wilson, J M, Hartley, R, Maxwell, D J, Todd, A J, Lieberam, I, Kaltschmidt, J A, et al. (2005). Conditional rhythmicity of ventral spinal interneurons defined by expression of the Hb9 homeodomain protein. The Journal of neuroscience: the official journal of the Society for Neuroscience 25: 5710-5719.
41. Kramer, E R, Knott, L, Su, F, Dessaud, E, Krull, C E, Helmbacher, F, et al. (2006). Cooperation between GDNF/Ret and ephrinA/EphA4 signals for motor-axon pathway selection in the limb. Neuron 50: 35-47.
42. Gogliotti, R G, Quinlan, K A, Barlow, C B, Heier, C R, Heckman, C J, and Didonato, C J (2012). Motor neuron rescue in spinal muscular atrophy mice demonstrates that sensory-motor defects are a consequence, not a cause, of motor neuron dysfunction. The Journal of neuroscience: the official journal of the Society for Neuroscience 32: 3818-3829.
43. Genc, B, Jara, J H, Lagrimas, A K, Pytel, P, Roos, R P, Mesulam, M M, et al. (2017). Apical dendrite degeneration, a novel cellular pathology for Betz cells in ALS. Sci Rep 7: 41765.
44. Chen, F, Sugiura, Y, Myers, K G, Liu, Y, and Lin, W (2010). Ubiquitin carboxyl-terminal hydrolase L1 is required for maintaining the structure and function of the neuromuscular junction. Proceedings of the National Academy of Sciences of the United States of America 107: 1636-1641.
45. Miura, H, Oda, K, Endo, C, Yamazaki, K, Shibasaki, H, and Kikuchi, T (1993). Progressive degeneration of motor nerve terminals in GAD mutant mouse with hereditary sensory axonopathy. Neuropathology and applied neurobiology 19: 41-51.
46. Desmond, N L, and Levy, W B (1986). Changes in the numerical density of synaptic contacts with long-term potentiation in the hippocampal dentate gyrus. The Journal of comparative neurology 253: 466-475.
47. Cartier, A E, Djakovic, S N, Salehi, A, Wilson, S M, Masliah, E, and Patrick, G N (2009). Regulation of synaptic structure by ubiquitin C-terminal hydrolase L1. The Journal of neuroscience: the official journal of the Society for Neuroscience 29: 7857-7868.
48. Gong, B, Cao, Z, Zheng, P, Vitolo, O V, Liu, S, Staniszewski, A, et al. (2006). Ubiquitin hydrolase Uch-L1 rescues beta-amyloid-induced decreases in synaptic function and contextual memory. Cell 126: 775-788.
49. Sakurai, M, Sekiguchi, M, Zushida, K, Yamada, K, Nagamine, S, Kabuta, T, et al. (2008). Reduction in memory in passive avoidance learning, exploratory behaviour and synaptic plasticity in mice with a spontaneous deletion in the ubiquitin C-terminal hydrolase L1 gene. The European journal of neuroscience 27: 691-701.
50. Chen, P C, Bhattacharyya, B J, Hanna, J, Minkel, H, Wilson, J A, Finley, D, et al. (2011). Ubiquitin homeostasis is critical for synaptic development and function. The Journal of neuroscience: the official journal of the Society for Neuroscience 31: 17505-17513.
51. Anderson, C T, Sheets, P L, Kiritani, T, and Shepherd, G M (2010). Sublayer-specific microcircuits of corticospinal and corticostriatal neurons in motor cortex. Nature neuroscience 13: 739-744.
52. Shepherd, G M (2013). Corticostriatal connectivity and its role in disease. Nature reviews Neuroscience 14: 278-291.
53. Yasvoina, M V, Genc, B, Jara, J H, Sheets, P L, Quinlan, K A, Milosevic, A, et al. (2013). eGFP expression under UCHL1 promoter genetically labels corticospinal motor neurons and a subpopulation of degeneration-resistant spinal motor neurons in an ALS mouse model. The Journal of neuroscience: the official journal of the Society for Neuroscience 33: 7890-7904.
54. Jara, J H, Genc, B, Stanford, M J, Pytel, P, Roos, R P, Weintraub, S, et al. (2017). Evidence for an early innate immune response in the motor cortex of ALS. J Neuroinflammation 14: 129.
55. Ozdinler, P H, Benn, S, Yamamoto, T H, Guzel, M, Brown, R H, Jr., and Macklis, J D (2011). Corticospinal motor neurons and related subcerebral projection neurons undergo early and specific neurodegeneration in hSOD1G(9)(3)A transgenic ALS mice. The Journal of neuroscience: the official journal of the Society for Neuroscience 31: 4166-4177.
56. Gautam, M, Jara, J H, Kocak, N, Rylaarsdam, L E, Kim, K D, Bigio, E H, et al. (2019). Mitochondria, E R, and nuclear membrane defects reveal early mechanisms for upper motor neuron vulnerability with respect to TDP-43 pathology. Acta neuropathologica 137: 47-69.
57. Wegorzewska, I, Bell, S, Cairns, N J, Miller, T M, and Baloh, R H (2009). TDP-43 mutant transgenic mice develop features of ALS and frontotemporal lobar degeneration. Proceedings of the National Academy of Sciences of the United States of America 106: 18809-18814.
58. Fogarty, M J, Klenowski, P M, Lee, J D, Drieberg-Thompson, J R, Bartlett, S E, Ngo, S T, et al. (2016). Cortical synaptic and dendritic spine abnormalities in a presymptomatic TDP-43 model of amyotrophic lateral sclerosis. Sci Rep 6: 37968.
59. Handley, E E, Pitman, K A, Dawkins, E, Young, K M, Clark, R M, Jiang, T C, et al. (2017). Synapse Dysfunction of Layer V Pyramidal Neurons Precedes Neurodegeneration in a Mouse Model of TDP-43 Proteinopathies. Cereb Cortex 27: 3630-3647.
60. Fil, D, DeLoach, A, Yadav, S, Alkam, D, MacNicol, M, Singh, A, et al. (2017). Mutant Profilin1 transgenic mice recapitulate cardinal features of motor neuron disease. Human molecular genetics 26: 686-701.
61. Gautam, M, Jara, J H, Sekerkova, G, Yasvoina, M V, Martina, M, and Ozdinler, P H (2016). Absence of alsin function leads to corticospinal motor neuron vulnerability via novel disease mechanisms. Human molecular genetics 25: 1074-1087.
62. Liu, H, Rose, M E, Ma, X, Culver, S, Dixon, C E, and Graham, S H (2017). In vivo transduction of neurons with TAT-UCH-L1 protects brain against controlled cortical impact injury. PloS one 12: e0178049.
63. Liu, H, Li, W, Ahmad, M, Miller, T M, Rose, M E, Poloyac, S M, et al. (2011). Modification of ubiquitin-C-terminal hydrolase-L1 by cyclopentenone prostaglandins exacerbates hypoxic injury. Neurobiology of disease 41: 318-328.
64. Poon, W W, Carlos, A J, Aguilar, B L, Berchtold, N C, Kawano, C K, Zograbyan, V, et al. (2013). beta-Amyloid (Abeta) oligomers impair brain-derived neurotrophic factor retrograde trafficking by down-regulating ubiquitin C-terminal hydrolase, UCH-L1. The Journal of biological chemistry 288: 16937-16948.
65. Vucic, S, and Kiernan, M C (2006). Novel threshold tracking techniques suggest that cortical hyperexcitability is an early feature of motor neuron disease. Brain: a journal of neurology 129: 2436-2446.
66. Menon, P, Kiernan, M C, and Vucic, S (2014). Biomarkers and future targets for development in amyotrophic lateral sclerosis. Curr Med Chem 21: 3535-3550.
67. Menon, P, Kiernan, M C, and Vucic, S (2014). Cortical excitability differences in hand muscles follow a split-hand pattern in healthy controls. Muscle & nerve 49: 836-844.
68. Vucic, S, Rothstein, J D, and Kiernan, M C (2014). Advances in treating amyotrophic lateral sclerosis: insights from pathophysiological studies. Trends in neurosciences 37: 433-442.
69. Geevasinga, N, Menon, P, Nicholson, G A, Ng, K, Howells, J, Kril, J J, et al. (2015). Cortical Function in Asymptomatic Carriers and Patients With C9orf72 Amyotrophic Lateral Sclerosis. JAMA Neurol 72: 1268-1274.
70. Joyce, P I, McGoldrick, P, Saccon, R A, Weber, W, Fratta, P, West, S J, et al. (2015). A novel SOD1-ALS mutation separates central and peripheral effects of mutant SOD1 toxicity. Human molecular genetics 24: 1883-1897.
71. Gong, S, Doughty, M, Harbaugh, C R, Cummins, A, Hatten, M E, Heintz, N, et al. (2007). Targeting Cre recombinase to specific neuron populations with bacterial artificial chromosome constructs. The Journal of neuroscience: the official journal of the Society for Neuroscience 27: 9817-9823.
72. Gong, S, Zheng, C, Doughty, M L, Losos, K, Didkovsky, N, Schambra, U B, et al. (2003). A gene expression atlas of the central nervous system based on bacterial artificial chromosomes. Nature 425: 917-925.
73. Lock, M, Alvira, M, Vandenberghe, L H, Samanta, A, Toelen, J, Debyser, Z, et al. (2010). Rapid, simple, and versatile manufacturing of recombinant adeno-associated viral vectors at scale. Hum Gene Ther 21: 1259-1271
74. Zhu, Y, Xu, J, Hauswirth, W W, and DeVries, S H (2014). Genetically targeted binary labeling of retinal neurons. The Journal of neuroscience: the official journal of the Society for Neuroscience 34: 7845-7861.
(2013). “Of men, not mice.” Nature Medicine 19(4): 379-379.
Ahmed, R. M., M. Irish, J. van Eersel, A. Ittner, Y. D. Ke, A. Volkerling, J. van der Hoven, K. Tanaka, T. Karl, M. Kassiou, J. J. Kril, O. Piguet, J. Gotz, M. C. Kiernan, G. M. Halliday, J. R. Hodges and L. M. Ittner (2017). “Mouse models of frontotemporal dementia: A comparison of phenotypes with clinical symptomatology.” Neurosci Biobehav Rev 74(Pt A): 126-138.
Bakavayev, S., N. Chetrit, T. Zvagelsky, R. Mansour, M. Vyazmensky, Z. Barak, A. Israelson and S. Engel (2019). “Cu/Zn-superoxide dismutase and wild-type like fALS SOD1 mutants produce cytotoxic quantities of H₂O₂ via cysteine-dependent redox short-circuit.” Scientific Reports 9(1): 10826.
Bigio, E. H. (2013). “Making the Diagnosis of Frontotemporal Lobar Degeneration.” Archives of Pathology & Laboratory Medicine 137(3): 314-325.
Chou, C. C., Y. Zhang, M. E. Umoh, S. W. Vaughan, I. Lorenzini, F. Liu, M. Sayegh, P. G. Donlin-Asp, Y. H. Chen, D. M. Duong, N. T. Seyfried, M. A. Powers, T. Kukar, C. M. Hales, M. Gearing, N. J. Cairns, K. B. Boylan, D. W. Dickson, R. Rademakers, Y. J. Zhang, L. Petrucelli, R. Sattler, D. C. Zarnescu, J. D. Glass and W. Rossoll (2018). “TDP-43 pathology disrupts nuclear pore complexes and nucleocytoplasmic transport in ALS/FTD.” Nature Neuroscience 21(2): 228-+.
Coan, G. and C. S. Mitchell (2015). “An Assessment of Possible Neuropathology and Clinical Relationships in 46 Sporadic Amyotrophic Lateral Sclerosis Patient Autopsies.” Neurodegener Dis 15(5): 301-312.
Cykowski, M. D., S. Z. Powell, L. E. Peterson, J. W. Appel, A. L. Rivera, H. Takei, E. Chang and S. H. Appel (2017). “Clinical Significance of TDP-43 Neuropathology in Amyotrophic Lateral Sclerosis.” Journal of Neuropathology and Experimental Neurology 76(5): 402-413.
Davis, S. A., S. Itaman, C. M. Khalid-Janney, J. A. Sherard, J. A. Dowell, N. J. Cairns and M. A. Gitcho (2018). “TDP-43 interacts with mitochondrial proteins critical for mitophagy and mitochondrial dynamics.” Neurosci Lett 678: 8-15.
Dawson, T. M., T. E. Golde and C. Lagier-Tourenne (2018). “Animal models of neurodegenerative diseases.” Nature Neuroscience 21(10): 1370-1379.
De Giorgio, F., C. Maduro, E. M. C. Fisher and A. Acevedo-Arozena (2019). “Transgenic and physiological mouse models give insights into different aspects of amyotrophic lateral sclerosis.” Disease Models & Mechanisms 12(1).
Dervishi, I. and P. H. Ozdinler (2018). “Incorporating upper motor neuron health in ALS drug discovery.” Drug Discovery Today 23(3): 696-703.
Fil, D., A. DeLoach, S. Yadav, D. Alkam, M. MacNicol, A. Singh, C. M. Compadre, J. J. Goellner, C. A. O'Brien, T. Fahmi, A. G. Basnakian, N. Y. Calingasan, J. L. Klessner, F. M. Beal, O. M. Peters, J. Metterville, R. H. Brown, Jr., K. K. Y. Ling, F. Rigo, P. H. Ozdinler and M. Kiaei (2017). “Mutant Profilin1 transgenic mice recapitulate cardinal features of motor neuron disease.” Hum Mol Genet 26(4): 686-701.
Fischer, L. R. and J. D. Glass (2010). “Oxidative stress induced by loss of Cu,Zn-superoxide dismutase (SOD1) or superoxide-generating herbicides causes axonal degeneration in mouse DRG cultures.” Acta Neuropathologica 119(2): 249-259.
Fisher, E. M. C. and D. M. Bannerman (2019). “Mouse models of neurodegeneration: Know your question, know your mouse.” Sci Transl Med 11(493).
Fogarty, M. J., P. M. Klenowski, J. D. Lee, J. R. Drieberg-Thompson, S. E. Bartlett, S. T. Ngo, M. A. Hilliard, M. C. Bellingham and P. G. Noakes (2016). “Cortical synaptic and dendritic spine abnormalities in a presymptomatic TDP-43 model of amyotrophic lateral sclerosis.” Scientific Reports 6: 37968.
Fogarty, M. J., E. W. H. Mu, P. G. Noakes, N. A. Lavidis and M. C. Bellingham (2016). “Marked changes in dendritic structure and spine density precede significant neuronal death in vulnerable cortical pyramidal neuron populations in the SOD1(G93A) mouse model of amyotrophic lateral sclerosis.” Acta Neuropathologica Communications 4(1): 77.
Fogarty, M. J., P. G. Noakes and M. C. Bellingham (2015). “Motor Cortex Layer V Pyramidal Neurons Exhibit Dendritic Regression, Spine Loss, and Increased Synaptic Excitation in the Presymptomatic hSOD1(G93A) Mouse Model of Amyotrophic Lateral Sclerosis.” Journal of Neuroscience 35(2): 643-647.
Gautam, M., J. H. Jara, N. Kocak, L. E. Rylaarsdam, K. D. Kim, E. H. Bigio and P. H. Ozdinler (2019). “Mitochondria, ER, and nuclear membrane defects reveal early mechanisms for upper motor neuron vulnerability with respect to TDP-43 pathology.” Acta Neuropathologica 137(1): 47-69.
Gautam, M., J. H. Jara, G. Sekerkova, M. V. Yasvoina, M. Martina and P. H. Ozdinler (2016). “Absence of alsin function leads to corticospinal motor neuron vulnerability via novel disease mechanisms.” Human Molecular Genetics 25(6): 1074-1087.
Gautam, M., E. F. Xie, N. Kocak and P. H. Ozdinler (2019). “Mitoautophagy: A Unique Self-Destructive Path Mitochondria of Upper Motor Neurons With TDP-43 Pathology Take, Very Early in ALS.” Frontiers in Cellular Neuroscience 13: 489.
Geevasinga, N., P. Menon, P. H. Ozdinler, M. C. Kiernan and S. Vucic (2016). “Pathophysiological and diagnostic implications of cortical dysfunction in ALS.” Nat Rev Neurol 12(11): 651-661.
Genc, B., O. Gozutok and P. H. Ozdinler (2019). “Complexity of Generating Mouse Models to Study the Upper Motor Neurons: Let Us Shift Focus from Mice to Neurons.” International Journal of Molecular Sciences 20(16).
Genc, B., J. H. Jara, A. K. B. Lagrimas, P. Pytel, R. P. Roos, M. M. Mesulam, C. Geula, E. H. Bigio and P. H. Ozdinler (2017). “Apical dendrite degeneration, a novel cellular pathology for Betz cells in ALS.” Scientific Reports 7: 41765.
Genc, B. and P. H. Ozdinler (2014). “Moving forward in clinical trials for ALS: motor neurons lead the way please.” Drug Discovery Today 19(4): 441-449.
Gurney, M. E. (1997). “The use of transgenic mouse models of amyotrophic lateral sclerosis in preclinical drug studies.” Journal of the Neurological Sciences 152: S67-S73.
Gurney, M. E., H. F. Pu, A. Y. Chiu, M. C. Dalcanto, C. Y. Polchow, D. D. Alexander, J. Caliendo, A. Hentati, Y. W. Kwon, H. X. Deng, W. J. Chen, P. Zhai, R. L. Sufit and T. Siddique (1994). “Motor-Neuron Degeneration in Mice That Express a Human Cu,Zn Superoxide-Dismutase Mutation.” Science 264(5166): 1772-1775.
Handley, E. E., K. A. Pitman, E. Dawkins, K. M. Young, R. M. Clark, T. C. C. Jiang, B. J. Turner, T. C. Dickson and C. A. Blizzard (2017). “Synapse Dysfunction of Layer V Pyramidal Neurons Precedes Neurodegeneration in a Mouse Model of TDP-43 Proteinopathies.” Cerebral Cortex 27(7): 3630-3647.
Haston, K. M. and S. Finkbeiner (2016). “Clinical Trials in a Dish: The Potential of Pluripotent Stem Cells to Develop Therapies for Neurodegenerative Diseases.” Annu Rev Pharmacol Toxicol 56: 489-510.
Hawrot, J., S. Imhof and B. J. Wainger (2020). “Modeling cell-autonomous motor neuron phenotypes in ALS using iPSCs.” Neurobiology of Disease 134: 104680.
Heyburn, L. and C. E. Moussa (2017). “TDP-43 in the spectrum of MND-FTLD pathologies.” Mol Cell Neurosci 83: 46-54.
Huynh, W., N. G. Simon, J. Grosskreutz, M. R. Turner, S. Vucic and M. C. Kiernan (2016). “Assessment of the upper motor neuron in amyotrophic lateral sclerosis.” Clinical Neurophysiology 127(7): 2643-2660.
Janus, C. and H. Welzl (2010). “Mouse Models of Neurodegenerative Diseases: Criteria and General Methodology.” Mouse Models for Drug Discovery: Methods and Protocols 602: 323-345.
Jara, J. H., M. Gautam, N. Kocak, E. F. Xie, Q. Mao, E. H. Bigio and P. H. Ozdinler (2019). “MCP1-CCR2 and neuroinflammation in the ALS motor cortex with TDP-43 pathology.” J Neuroinflammation 16(1): 196.
Jara, J. H., B. Gene, G. A. Cox, M. C. Bohn, R. P. Roos, J. D. Macklis, E. Ulupinar and P. H. Ozdinler (2015). “Corticospinal Motor Neurons Are Susceptible to Increased ER Stress and Display Profound Degeneration in the Absence of UCHL1 Function.” Cereb Cortex 25(11): 4259-4272.
Jara, J. H., B. Genc, J. L. Klessner and P. H. Ozdinler (2014). “Retrograde labeling, transduction, and genetic targeting allow cellular analysis of corticospinal motor neurons: implications in health and disease.” Front Neuroanat 8: 16.
Jara, J. H., S. R. Villa, N. A. Khan, M. C. Bohn and P. H. Ozdinler (2012). “AAV2 mediated retrograde transduction of corticospinal motor neurons reveals initial and selective apical dendrite degeneration in ALS.” Neurobiol Dis 47(2): 174-183.
Julien, J. P. and J. Kriz (2006). “Transgenic mouse models of amyotrophic lateral sclerosis.” Biochim Biophys Acta 1762(11-12): 1013-1024.
Kim, H. J. and J. P. Taylor (2017). “Lost in Transportation: Nucleocytoplasmic Transport Defects in ALS and Other Neurodegenerative Diseases.” Neuron 96(2): 285-297.
Li, Q., C. Vande Velde, A. Israelson, J. Xie, A. O. Bailey, M. Q. Dong, S. J. Chun, T. Roy, L. Winer, J. R. Yates, R. A. Capaldi, D. W. Cleveland and T. M. Miller (2010). “ALS-linked mutant superoxide dismutase 1 (SOD1) alters mitochondrial protein composition and decreases protein import.” Proc Natl Acad Sci USA 107(49): 21146-21151.
Ling, S. C., M. Polymenidou and D. W. Cleveland (2013). “Converging mechanisms in ALS and FTD: disrupted RNA and protein homeostasis.” Neuron 79(3): 416-438.
Lutz, C. (2018). “Mouse models of ALS: Past, present and future.” Brain Research 1693(Pt A): 1-10.
Mackenzie, I. R. A., E. H. Bigio, P. G. Ince, F. Geser, M. Neumann, N. J. Cairns, L. K. Kwong, M. S. Forman, J. Ravits, H. Stewart, A. Eisen, L. Mcclusky, H. A. Kretzschmar, C. M. Monoranu, J. R. Highley, J. Kirby, T. Siddique, P. J. Shaw, V. M. Y. Lee and J. Q. Trojanowski (2007). “Pathological TDP-43 distinguishes sporadic amyotrophic lateral sclerosis from amyotrophic lateral sclerosis with SOD1 mutations.” Annals of Neurology 61(5): 427-434.
Menon, P., N. Geevasinga, C. Yiannikas, J. Howells, M. C. Kiernan and S. Vucic (2015). “Sensitivity and specificity of threshold tracking transcranial magnetic stimulation for diagnosis of amyotrophic lateral sclerosis: a prospective study.” The Lancet Neurology 14(5): 478-484.
Neumann, M., D. M. Sampathu, L. K. Kwong, A. C. Truax, M. C. Micsenyi, T. T. Chou, J. Bruce, T. Schuck, M. Grossman, C. M. Clark, L. F. McCluskey, B. L. Miller, E. Masliah, I. R. Mackenzie, H. Feldman, W. Feiden, H. A. Kretzschmar, J.
Q. Trojanowski and V. M. Lee (2006). “Ubiquitinated TDP-43 in frontotemporal lobar degeneration and amyotrophic lateral sclerosis.” Science 314(5796): 130-133.
Ozdinler, P. H., S. Benn, T. H. Yamamoto, M. Guzel, R. H. Brown and J. D. Macklis (2011). “Corticospinal Motor Neurons and Related Subcerebral Projection Neurons Undergo Early and Specific Neurodegeneration in hSOD1G93A Transgenic ALS Mice.” Journal of Neuroscience 31(11): 4166-4177.
Ozdinler, P. H. and J. D. Macklis (2006). “IGF-I specifically enhances axon outgrowth of corticospinal motor neurons.” Nature Neuroscience 9(11): 1371-1381.
Ozdinler, P. H. and R. B. Silverman (2014). “Treatment of amyotrophic lateral sclerosis: lessons learned from many failures.” ACS Med Chem Lett 5(11): 1179-1181.
Pasinelli, P., M. E. Belford, N. Lennon, B. J. Bacskai, B. T. Hyman, D. Trotti and R. H. Brown, Jr. (2004). “Amyotrophic lateral sclerosis-associated SOD1 mutant proteins bind and aggregate with Bcl-2 in spinal cord mitochondria.” Neuron 43(1): 19-30.
Perrin, S. (2014). “Preclinical research: Make mouse studies work.” Nature 507(7493): 423-425.
Philips, T. and J. D. Rothstein (2015). “Rodent Models of Amyotrophic Lateral Sclerosis.” Curr Protoc Pharmacol 69: 5 67 61-65 67 21.
Pickles, S., L. Destroismaisons, S. L. Peyrard, S. Cadot, G. A. Rouleau, R. H. Brown, J. P. Julien, N. Arbour and C. Vande Velde (2013). “Mitochondrial damage revealed by immunoselection for ALS-linked misfolded SOD1.” Human Molecular Genetics 22(19): 3947-3959.
Pickles, S., S. Semmler, H. R. Broom, L. Destroismaisons, L. Legroux, N. Arbour, E. Meiering, N. R. Cashman and C. Vande Velde (2016). “ALS-linked misfolded SOD1 species have divergent impacts on mitochondria.” Acta Neuropathol Commun 4(1): 43.
Pollari, E., G. Goldsteins, G. Bart, J. Koistinaho and R. Giniatullin (2014). “The role of oxidative stress in degeneration of the neuromuscular junction in amyotrophic lateral sclerosis.” Frontiers in Cellular Neuroscience 8: 131.
Ransohoff, R. M. (2018). “All (animal) models (of neurodegeneration) are wrong. Are they also useful?” Journal of Experimental Medicine 215(12): 2955-2958.
Robertson, J., T. Sanelli, S. X. Xiao, W. C. Yang, P. Horne, R. Hammond, E. P. Pioro and M. J. S. Trong (2007). “Lack of TDP-43 abnormalities in mutant SOD1 transgenic mice shows disparity with ALS.” Neuroscience Letters 420(2): 128-132.
Rosen, D. R., T. Siddique, D. Patterson, D. A. Figlewicz, P. Sapp, A. Hentati, D. Donaldson, J. Goto, J. P. O'Regan, H.-X. Deng, Z. Rahmani, A. Krizus, D. McKenna-Yasek, A. Cayabyab, S. M. Gaston, R. Berger, R. E. Tanzi, J. J. Halperin, B. Herzfeldt, R. Van den Bergh, W.-Y. Hung, T. Bird, G. Deng, D. W. Mulder, C. Smyth, N. G. Laing, E. Soriano, M. A. Pericak-Vance, J. Haines, G. A. Rouleau, J. S. Gusella, H. R. Horvitz and R. H. Brown (1993). “Mutations in Cu/Zn superoxide dismutase gene are associated with familial amyotrophic lateral sclerosis.” Nature 362(6415): 59-62.
Tan, W., P. Pasinelli and D. Trotti (2014). “Role of mitochondria in mutant SOD1 linked amyotrophic lateral sclerosis.” Biochim Biophys Acta 1842(8): 1295-1301.
Thomsen, G. M., G. Gowing, S. Svendsen and C. N. Svendsen (2014). “The past, present and future of stem cell clinical trials for ALS.” Experimental Neurology 262: 127-137.
Vucic, S., U. Ziemann, A. Eisen, M. Hallett and M. C. Kiernan (2013). “Transcranial magnetic stimulation and amyotrophic lateral sclerosis: pathophysiological insights.” J Neurol Neurosurg Psychiatry 84(10): 1161-1170.
Wang, W., L. Wang, J. Lu, S. L. Siedlak, H. Fujioka, J. Liang, S. Jiang, X. Ma, Z. Jiang, E. L. da Rocha, M. Sheng, H. Choi, P. H. Lerou, H. Li and X. Wang (2016). “The inhibition of TDP-43 mitochondrial localization blocks its neuronal toxicity.” Nat Med 22(8): 869-878.
Wang, W. Z., L. Li, W. L. Lin, D. W. Dickson, L. Petrucelli, T. Zhang and X. L. Wang (2013). “The ALS disease-associated mutant TDP-43 impairs mitochondrial dynamics and function in motor neurons.” Human Molecular Genetics 22(23): 4706-4719.
Wegorzewska, I. and R. H. Baloh (2011). “TDP-43-Based Animal Models of Neurodegeneration: New Insights into ALS Pathology and Pathophysiology.” Neurodegenerative Diseases 8(4): 262-274.
Wegorzewska, I., S. Bell, N. J. Cairns, T. M. Miller and R. H. Baloh (2009). “TDP-43 mutant transgenic mice develop features of ALS and frontotemporal lobar degeneration.” Proc Natl Acad Sci USA 106(44): 18809-18814.
Yasvoina, M. V., B. Genc, J. H. Jara, P. L. Sheets, K. A. Quinlan, A. Milosevic, G. M. G. Shepherd, C. J. Heckman and P. H. Ozdinler (2013). “eGFP Expression under UCHL1 Promoter Genetically Labels Corticospinal Motor Neurons and a Subpopulation of Degeneration-Resistant Spinal Motor Neurons in an ALS Mouse Model.” Journal of Neuroscience 33(18): 7890-7904.
Zhu, Y., J. Xu, W. W. Hauswirth and S. H. DeVries (2014). “Genetically targeted binary labeling of retinal neurons.” J Neurosci 34(23): 7845-7861.

pAAV-hUCHL1P-UCHL1-IRES-mCherry
(SEQ ID NO: 7)
GC%: 52.60%
Sequence:
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGCA
CGCGTTGACATTGATTATTGACTAGTTGGAGCCCAGTTTAGCAGGGTTTA
CTTCTCAGTTTTACTTTTCAATTGTGACCGTAAGCAAATTAACTTCTCTA
GGCCTGGGATTCTGATCTGTAAAATTGCACTAATATGAGTCTCTTCACGG
CTCCTCTAAGGATTAAATGAGAGACACATGCAAAGGATCCCCAAAAACAA
TAACTCAAAAAATGTTGATTCCCTCCCTTCCCTCTGTCATCTGTTAACCT
CAACTTCCTAAATAGAAGGTCTATTCTTTTACCATCATCATTATTCTCTT
CGGTGCCTATTTTTAAAAAATACTCAACCTTCTTGCTTCCTTCGCTACCT
AAGTATTTCTGCAAGCCCACTTTGTTCTGCAGCTTAGCTTTCCTGGCACA
ATTCTTATAGATTTTGGTCCCTTTTAAAATTCATTCTTCAGCAAATGCTT
TCTCTCTCCATCTTTTGACTAGAGATCATTAGAGATCACCTGAGATCATT
AGAGAACAGTGGTTTCCTTGGTTGCCATTCCCTTTCTTCTTCATTGGGAG
TATTCTGCGGTGAACTCAGACATTTTATTTTTCAAAGCTTCCCATTCTTT
TAAAAATGCTTTTCCTTTTACAGCCTCTCGCTCAAAATCATACCCATCTT
TTCCCTGGATCTGTTTTCTCAAGTCTCCAATCGCCTGCCTTCTTTGTGTC
TTGTATTACCCTCACATCCCCCAGCTTTCTACTGCTCTCCCAGGACCAAC
CATTTCTTCCGCGGGAGTCACATTACATCAGCATTCCTAATGCAGTATCT
GTTATCTACCAGATTCTGTTTTATTCTAGGTAGTCACTTAAAAACGAACC
TCGGTACTGGTCTGACTTAACATGGAGGAGGAATTGTCTAAGGTTAAACG
CAAACTGCTGAGAGATTTGGGGCGGGGGGCACACATTTACATTCATTCGT
ATTAAATATATACCTGTTGAATTTGTGCTTTTTCTCAAATGCTTCAGAGA
CTCGAGCTTTAGAGTAATTGGGATGGTGAAAGGATGGGTTTCCAGAAACT
TCGCCCAAAATTAAAGACTCCATCAAAAGGACTGCTCCATACACTCAAGG
AACACCCACCAACAAATCCCGTCTCCACAACCACCAGATTATCTCACCGG
CGAGTGAGACTGCAAGGTTTGGGGGCCCGGCCGTACCACTCCGCGCTGCG
CACGGGGGGTTCGTACCCATCTGGCCGCGACCGTCCGTTTCCCCCTCGCT
TGGTTCTGCCCCTGCTCCCCCTGCACAGGCCTCACAGTGCGTCTGGCCGG
CGCTTTATAGCTGCAGCCTGGGCGACCGGATCACAAGTTTGTACAAAAAA
GCAGGCTCCGCGGCCGCCCCCTTCACCATGCAGCTGAAGCCGATGGAGAT
TAACCCCGAGATGCTGAACAAAGTGTTGGCCAAGCTGGGGGTCGCCGGCC
AGTGGCGCTTCGCCGACGTGCTAGGGCTGGAGGAGGAGACTCTGGGCTCA
GTGCCATCCCCTGCCTGCGCCCTGCTGCTCCTGTTTCCCCTCACGGCCCA
GCATGAAAACTTCAGGAAAAAGCAAATTGAGGAACTGAAGGGACAGGAAG
TTAGCCCTAAAGTTTACTTCATGAAGCAGACCATCGGAAACTCCTGTGGT
ACCATCGGGTTGATCCACGCAGTGGCCAACAACCAAGACAAGCTGGAATT
TGAGGATGGATCCGTCCTGAAACAGTTTCTGTCTGAAACGGAGAAGCTGT
CCCCCGAAGATAGAGCCAAGTGTTTCGAGAAGAACGAGGCCATCCAGGCA
GCCCATGACTCCGTGGCCCAGGAGGGCCAGTGTCGGGTAGATGACAAAGT
GAATTTCCATTTTATTCTGTTCAACAACGTGGACGGCCATCTGTACGAGC
TCGATGGGCGAATGCCCTTTCCAGTGAACCATGGCGCCAGCTCAGAGGAC
TCTCTGCTGCAGGATGCTGCCAAGGTCTGCAGAGAATTCACTGAGCGCGA
GCAGGGGGAGGTCCGCTTCTCTGCCGTGGCTCTCTGCAAAGCAGCTTAAA
ICAGATCCAAGGGTGGGCGCGCCGACCCAGCTTTCTTGTACAAAGTGGTG
ATCCGGTGCTAGCCTCGAGAATTCACGCGTCGAGCATGCATCTAGGGCGG
CCAATTCCGCCCCTCTCCCTCCCCCCCCCCTAACGTTACTGGCCGAAGCC
GCTTGGAATAAGGCCGGTGTGCGTTTGTCTATATGTGATTTTCCACCATA
TTGCCGTCTTTTGGCAATGTGAGGGCCCGGAAACCTGGCCCTGTCTTCTT
GACGAGCATTCCTAGGGGTCTTTCCCCTCTCGCCAAAGGAATGCAAGGTC
TGTTGAATGTCGTGAAGGAAGCAGTTCCTCTGGAAGCTTCTTGAAGACAA
ACAACGTCTGTAGCGACCCTTTGCAGGCAGCGGAACCCCCCACCTGGCGA
CAGGTGCCTCTGCGGCCAAAAGCCACGTGTATAAGATACACCTGCAAAGG
CGGCACAACCCCAGTGCCACGTTGTGAGTTGGATAGTTGTGGAAAGAGTC
AAATGGCTCTCCTCAAGCGTATTCAACAAGGGGCTGAAGGATGCCCAGAA
GGTACCCCATTGTATGGGATCTGATCTGGGGCCTCGGTGCACATGCTTTA
CATGTGTTTAGTCGAGGTTAAAAAAACGTCTAGGCCCCCCGAACCACGGG
GACGTGGTTTTCCTTTGAAAAACACGATGATAAGCTTGCCACAACCCGGG
ATCCACCATGGTGAGCAAGGGCGAGGAGGATAACATGGCCATCATCAAGG
AGTTCATGCGCTTCAAGGTGCACATGGAGGGCTCCGTGAACGGCCACGAG
TTCGAGATCGAGGGCGAGGGCGAGGGCCGCCCCTACGAGGGCACCCAGAC
CGCCAAGCTGAAGGTGACCAAGGGTGGCCCCCTGCCCTTCGCCTGGGACA
TCCTGTCCCCTCAGTTCATGTACGGCTCCAAGGCCTACGTGAAGCACCCC
GCCGACATCCCCGACTACTTGAAGCTGTCCTTCCCCGAGGGCTTCAAGTG
GGAGCGCGTGATGAACTTCGAGGACGGCGGCGTGGTGACCGTGACCCAGG
ACTCCTCCCTGCAGGACGGCGAGTTCATCTACAAGGTGAAGCTGCGCGGC
ACCAACTTCCCCTCCGACGGCCCCGTAATGCAGAAGAAGACCATGGGCTG
GGAGGCCTCCTCCGAGCGGATGTACCCCGAGGACGGCGCCCTGAAGGGCG
AGATCAAGCAGAGGCTGAAGCTGAAGGACGGCGGCCACTACGACGCTGAG
GTCAAGACCACCTACAAGGCCAAGAAGCCCGTGCAGCTGCCCGGCGCCTA
CAACGTCAACATCAAGTTGGACATCACCTCCCACAACGAGGACTACACCA
TCGTGGAACAGTACGAACGCGCCGAGGGCCGCCACTCCACCGGCGGCATG
GACGAGCTGTACAAGTAATGAATTCGCTTATCGATAATCAACCTCTGGAT
TACAAAATTTGTGAAAGATTGACTGGTATTCTTAACTATGTTGCTCCTTT
TACGCTATGTGGATACGCTGCTTTAATGCCTTTGTATCATGCTATTGCTT
CCCGTATGGCTTTCATTTTCTCCTCCTTGTATAAATCCTGGTTGCTGTCT
CTTTATGAGGAGTTGTGGCCCGTTGTCAGGCAACGTGGCGTGGTGTGCAC
TGTGTTTGCTGACGCAACCCCCACTGGTTGGGGCATTGCCACCACCTGTC
AGCTCCTTTCCGGGACTTTCGCTTTCCCCCTCCCTATTGCCACGGCGGAA
CTCATCGCCGCCTGCCTTGCCCGCTGCTGGACAGGGGCTCGGCTGTTGGG
CACTGACAATTCCGTGGTGTTGTCGGGGAAATCATCGTCCTTTCCTTGGC
TGCTCGCCTGTGTTGCCACCTGGATTCTGCGCGGGACGTCCTTCTGCTAC
GTCCCTTCGGCCCTCAATCCAGCGGACCTTCCTTCCCGCGGCCTGCTGCC
GGCTCTGCGGCCTCTTCCGCGTCTTCGCCTTCGCCCTCAGACGAGTCGGA
TCTCCCTTTGGGCCGCCTCCCCGCATCGATACCGAGCGCTGCTCGAGAGA
TCTACGGGTGGCATCCCTGTGACCCCTCCCCAGTGCCTCTCCTGGCCCTG
GAAGTTGCCACTCCAGTGCCCACCAGCCTTGTCCTAATAAAATTAAGTTG
CATCATTTTGTCTGACTAGGTGTCCTTCTATAATATTATGGGGTGGAGGG
GGGTGGTATGGAGCAAGGGGCAAGTTGGGAAGACAACCTGTAGGGCCTGC
GGGGTCTATTGGGAACCAAGCTGGAGTGCAGTGGCACAATCTTGGCTCAC
TGCAATCTCCGCCTCCTGGGTTCAAGCGATTCTCCTGCCTCAGCCTCCCG
AGTTGTTGGGATTCCAGGCATGCATGACCAGGCTCAGCTAATTTTTGTTT
TTTTGGTAGAGACGGGGTTTCACCATATTGGCCAGGCTGGTCTCCAACTC
CTAATCTCAGGTGATCTACCCACCTTGGCCTCCCAAATTGCTGGGATTAC
AGGCGTGAACCACTGCTCCCTTCCCTGTCCTTCTGATTTTGTAGGTAACC
ACGTGCGGACCGAGCGGCCGCAGGAACCCCTAGTGATGGAGTTGGCCACT
CCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCA
GCTGCCTGCAGGGGCGCCTGATGCGGTATTTTCTCCTTACGCATCTGTGC
GGTATTTCACACCGCATACGTCAAAGCAACCATAGTACGCGCCCTGTAGC
GGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTC
TCGCCACGTTCGCCGGCTTTCCCCGTCAAGCTCTAAATCGGGGGCTCCCT
TTAGGGTTCCGATTTAGTGCTTTACGGCACCTCGACCCCAAAAAACTTGA
TTTGGGTGATGGTTCACGTAGTGGGCCATCGCCCTGATAGACGGTTTTTC
GCCCTTTGACGTTGGAGTCCACGTTCTTTAATAGTGGACTCTTGTTCCAA
ACTGGAACAACACTCAACCCTATCTCGGGCTATTCTTTTGATTTATAAGG
GATTTTGCCGATTTCGGCCTATTGGTTAAAAAATGAGCTGATTTAACAAA
AATTTAACGCGAATTTTAACAAAATATTAACGTTTACAATTTTATGGTGC
ACTCTCAGTACAATCTGCTCTGATGCCGCATAGTTAAGCCAGCCCCGACA
CCCGCCAACACCCGCTGACGCGCCCTGACGGGCTTGTCTGCTCCCGGCAT
CCGCTTACAGACAAGCTGTGACCGTCTCCGGGAGCTGCATGTGTCAGAGG
TTTTCACCGTCATCACCGAAACGCGCGAGACGAAAGGGCCTCGTGATACG
CCTATTTTTATAGGTTAATGTCATGATAATAATGGTTTCTTAGACGTCAG
GTGGCACTTTTCGGGGAAATGTGCGCGGAACCCCTATTTGTTTATTTTTC
TAAATACATTCAAATATGTATCCGCTCATGAGACAATAACCCTGATAAAT
GCTTCAATAATATTGAAAAAGGAAGAGTATGAGTATTCAACATTTCCGTG
TCGCCCTTATTCCCTTTTTTGCGGCATTTTGCCTTCCTGTTTTTGCTCAC
CCAGAAACGCTGGTGAAAGTAAAAGATGCTGAAGATCAGTTGGGTGCACG
AGTGGGTTACATCGAACTGGATCTCAACAGCGGTAAGATCCTTGAGAGTT
TTCGCCCCGAAGAACGTTTTCCAATGATGAGCACTTTTAAAGTTCTGCTA
TGTGGCGCGGTATTATCCCGTATTGACGCCGGGCAAGAGCAACTCGGTCG
CCGCATACACTATTCTCAGAATGACTTGGTTGAGTACTCACCAGTCACAG
AAAAGCATCTTACGGATGGCATGACAGTAAGAGAATTATGCAGTGCTGCC
ATAACCATGAGTGATAACACTGCGGCCAACTTACTTCTGACAACGATCGG
AGGACCGAAGGAGCTAACCGCTTTTTTGCACAACATGGGGGATCATGTAA
CTCGCCTTGATCGTTGGGAACCGGAGCTGAATGAAGCCATACCAAACGAC
GAGCGTGACACCACGATGCCTGTAGCAATGGCAACAACGTTGCGCAAACT
ATTAACTGGCGAACTACTTACTCTAGCTTCCCGGCAACAATTAATAGACT
GGATGGAGGCGGATAAAGTTGCAGGACCACTTCTGCGCTCGGCCCTTCCG
GCTGGCTGGTTTATTGCTGATAAATCTGGAGCCGGTGAGCGTGGGTCTCG
CGGTATCATTGCAGCACTGGGGCCAGATGGTAAGCCCTCCCGTATCGTAG
TTATCTACACGACGGGGAGTCAGGCAACTATGGATGAACGAAATAGACAG
ATCGCTGAGATAGGTGCCTCACTGATTAAGCATTGGTAACTGTCAGACCA
AGTTTACTCATATATACTTTAGATTGATTTAAAACTTCATTTTTAATTTA
AAAGGATCTAGGTGAAGATCCTTTTTGATAATCTCATGACCAAAATCCCT
TAACGTGAGTTTTCGTTCCACTGAGCGTCAGACCCCGTAGAAAAGATCAA
AGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTG
TAGCACCGCCTACATACCTCGCTCTGCTAATCCTGTTACCAGTGGCTGCT
GCCAGTGGCGATAAGTCGTGTCTTACCGGGTTGGACTCAAGACGATAGTT
ACCGGATAAGGCGCAGCGGTCGGGCTGAACGGGGGGTTCGTGCACACAGC
CCAGCTTGGAGCGAACGACCTACACCGAACTGAGATACCTACAGCGTGAG
CTATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGGG
GAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAA
CGCCAGCAACGCGGCCTTTTTACGGTTCCTGGCCTTTTGCTGGCCTTTTG
CTCACATGT
pAAV-CBA-UCHL1-IRES-mCherry
(SEQ ID NO: 8)
CCTGCAGGCAGCTGCGCGCTCGCTCGCTCACTGAGGCCGCCCGGGCAAAGCCCGG
GCGTCGGGCGACCTTTGGTCGCCCGGCCTCAGTGAGCGAGCGAGCGCGCAGAGAG
GGAGTGGCCAACTCCATCACTAGGGGTTCCTGCGGCCGCACGCGTTGACATTGAT
TATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCC
AACGACCCCCGCCCATTGACGTCAATAATGACGTATGTTCCCATAGTAACGCCAA
TAGGGACTTTCCATTGACGTCAATGGGTGGACTATTTACGGTAAACTGCCCACTT
GGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTA
CTTGGCAGTACATCTACGTATTAGTCATCGCTATTACCATGGGTCGAGGTGAGCC
CCACGTTCTGCTTCACTCTCCCCATCTCCCCCCCCTCCCCACCCCCAATTTTGTA
TTTATTTATTTTTTAATTATTTTGTGCAGCGATGGGGGCGGGGGGGGGGGGGGCG
CGCGCCAGGCGGGGCGGGGCGGGGCGAGGGGCGGGGCGGGGCGAGGCGGAGAGGT
GCGGCGGCAGCCAATCAGAGCGGCGCGCTCCGAAAGTTTCCTTTTATGGCGAGGC
GGCGGCGGCGGCGGCCCTATAAAAAGCGAAGCGCGCGGCGGGCGGGAGTCGCTGC
GTTGCCTTCGCCCCGTGCCCCGCTCCGCGCCGCCTCGCGCCGCCCGCCCCGGCTC
TGACTGACCGCGTTACTCCCACAGGTGAGCGGGCGGGACGGCCCTTCTCCTCCGG
GCTGTAATTAGCGCTTGGTTTAATGACGGCTCGTTTCTTTTCTGTGGCTGCGTGA
AAGCCTTAAAGGGCTCCGGGAGGGCCCTTTGTGCGGGGGGGAGCGGCTCGGGGGG
TGCGTGCGTGTGTGTGTGCGTGGGGAGCGCCGCGTGCGGCCCGCGCTGCCCGGCG
GCTGTGAGCGCTGCGGGCGCGGCGCGGGGCTTTGTGCGCTCCGCGTGTGCGCGAG
GGGAGCGCGGCCGGGGGCGGTGCCCCGCGGTGCGGGGGGGCTGCGAGGGGAACAA
AGGCTGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCCCG
GCTTCGGGTGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGGGCCGGGGA
GGGCTCGGGGGAGGGGCGCGGCGGCCCCGGAGCGCCGGCGGCTGTCGAGGCGCGG
CGAGCCGCAGCCATTGCCTTTTATGGTAATCGTGCGAGAGGGCGCAGGGACTTCC
TTTGTCCCAAATCTGGCGGAGCCGAAATCTGGGAGGCGCCGCCGCACCCCCTCTA
GCGGGCGCGGGCGAAGCGGTGCGGCGCCGGCAGGAAGGAAATGGGCGGGGAGGGC
CTTCGTGCGTCGCCGCGCCGCCGTCCCCTTCTCCATCTCCAGCCTCGGGGCTGCC
GCAGGGGGACGGCTGCCTTCGGGGGGGACGGGGCAGGGCGGGGTTCGGCTTCTGG
CGTGTGACCGGCGGCTCTAGAGCCTCTGCTAACCATGTTCATGCCTTCTTCTTTT
TCCTACAGCTCCTGGGCAACGTGCTGGTTATTGTGCTGTCTCATCATTTTGGCAA
AGAATTCTGCAGTCGAATCACAAGTTTGTACAAAAAAGCTGAACGAGAAACGTAA
AATGATATAAATATCAATATATTAAATTAGATTTTGCATAAAAAACAGACTACAT
AATACTGTAAAACACAACATATCCAGTCACTATGGCGGCCGCATTAGGCACCCCA
GGCTTTACACTTTATGCTTCCGGCTCGTATAATGTGTGGATTTTGAGTTAGGATC
CGTCGAGATTTTCAGGAGCTAAGGAAGCTAAAATGGAGAAAAAAATCACTGGATA
TACCACCGTTGATATATCCCAATGGCATCGTAAAGAACATTTTGAGGCATTTCAG
TCAGTTGCTCAATGTACCTATAACCAGACCGTTCAGCTGGATATTACGGCCTTTT
TAAAGACCGTAAAGAAAAATAAGCACAAGTTTTATCCGGCCTTTATTCACATTCT
TGCCCGCCTGATGAATGCTCATCCGGAATTCCGTATGGCAATGAAAGACGGTGAG
CTGGTGATATGGGATAGTGTTCACCCTTGTTACACCGTTTTCCATGAGCAAACTG
AAACGTTTTCATCGCTCTGGAGTGAATACCACGACGATTTCCGGCAGTTTCTACA
CATATATTCGCAAGATGTGGCGTGTTACGGTGAAAACCTGGCCTATTTCCCTAAA
GGGTTTATTGAGAATATGTTTTTCGTCTCAGCCAATCCCTGGGTGAGTTTCACCA
GTTTTGATTTAAACGTGGCCAATATGGACAACTTCTTCGCCCCCGTTTTCACCAT
GGGCAAATATTATACGCAAGGCGACAAGGTGCTGATGCCGCTGGCGATTCAGGTT
CATCATGCCGTTTGTGATGGCTTCCATGTCGGCAGAATGCTTAATGAATTACAAC
AGTACTGCGATGAGTGGCAGGGCGGGGCGTAAACGCGTGGATCCGGCTTACTAAA
AGCCAGATAACAGTATGCGTATTTGCGCGCTGATTTTTGCGGTATAAGAATATAT
ACTGATATGTATACCCGAAGTATGTCAAAAAGAGGTATGCTATGAAGCAGCGTAT
TACAGTGACAGTTGACAGCGACAGCTATCAGTTGCTCAAGGCATATATGATGTCA
ATATCTCCGGTCTGGTAAGCACAACCATGCAGAATGAAGCCCGTCGTCTGCGTGC
CGAACGCTGGAAAGCGGAAAATCAGGAAGGGATGGCTGAGGTCGCCCGGTTTATT
GAAATGAACGGCTCTTTTGCTGACGAGAACAGGGGCTGGTGAAATGCAGTTTAAG
GTTTACACCTATAAAAGAGAGAGCCGTTATCGTCTGTTTGTGGATGTACAGAGTG
ATATTATTGACACGCCCGGGCGACGGATGGTGATCCCCCTGGCCAGTGCACGTCT
GCTGTCAGATAAAGTCTCCCGTGAACTTTACCCGGTGGTGCATATCGGGGATGAA
AGCTGGCGCATGATGACCACCGATATGGCCAGTGTGCCGGTCTCCGTTATCGGGG
AAGAAGTGGCTGATCTCAGCCACCGCGAAAATGACATCAAAAACGCCATTAACCT
GATGTTCTGGGGAATATAAATGTCAGGCTCCCTTATACACAGCCAGTCTGCAGGT
CGACCATAGTGACTGGATATGTTGTGTTTTACAGTATTATGTAGTCTGTTTTTTA
TGCAAAATCTAATTTAATATATTGATATTTATATCATTTTACGTTTCTCGTTCAG
CTTTCTTGTACAAAGTGGTGATCTAGCCTCGAGAATTCACGCGTCGAGCATGCAT
CTAGGGCGGCCAATTCCGCCCCTCTCCCTCCCCCCCCCCTAACGTTACTGGCCGA
AGCCGCTTGGAATAAGGCCGGTGTGCGTTTGTCTATATGTGATTTTCCACCATAT
TGCCGTCTTTTGGCAATGTGAGGGCCCGGAAACCTGGCCCTGTCTTCTTGACGAG
CATTCCTAGGGGTCTTTCCCCTCTCGCCAAAGGAATGCAAGGTCTGTTGAATGTC
GTGAAGGAAGCAGTTCCTCTGGAAGCTTCTTGAAGACAAACAACGTCTGTAGCGA
CCCTTTGCAGGCAGCGGAACCCCCCACCTGGCGACAGGTGCCTCTGCGGCCAAAA
GCCACGTGTATAAGATACACCTGCAAAGGCGGCACAACCCCAGTGCCACGTTGTG
AGTTGGATAGTTGTGGAAAGAGTCAAATGGCTCTCCTCAAGCGTATTCAACAAGG
GGCTGAAGGATGCCCAGAAGGTACCCCATTGTATGGGATCTGATCTGGGGCCTCG
GTGCACATGCTTTACATGTGTTTAGTCGAGGTTAAAAAAACGTCTAGGCCCCCCG
AACCACGGGGACGTGGTTTTCCTTTGAAAAACACGATGATAAGCTTGCCACAACC
CGGGATCCACCATGGTGAGCAAGGGCGAGGAGGATAACATGGCCATCATCAAGGA
GTTCATGCGCTTCAAGGTGCACATGGAGGGCTCCGTGAACGGCCACGAGTTCGAG
ATCGAGGGCGAGGGCGAGGGCCGCCCCTACGAGGGCACCCAGACCGCCAAGCTGA
AGGTGACCAAGGGTGGCCCCCTGCCCTTCGCCTGGGACATCCTGTCCCCTCAGTT
CATGTACGGCTCCAAGGCCTACGTGAAGCACCCCGCCGACATCCCCGACTACTTG
AAGCTGTCCTTCCCCGAGGGCTTCAAGTGGGAGCGCGTGATGAACTTCGAGGACG
GCGGCGTGGTGACCGTGACCCAGGACTCCTCCCTGCAGGACGGCGAGTTCATCTA
CAAGGTGAAGCTGCGCGGCACCAACTTCCCCTCCGACGGCCCCGTAATGCAGAAG
AAGACCATGGGCTGGGAGGCCTCCTCCGAGCGGATGTACCCCGAGGACGGCGCCC
TGAAGGGCGAGATCAAGCAGAGGCTGAAGCTGAAGGACGGCGGCCACTACGACGC
TGAGGTCAAGACCACCTACAAGGCCAAGAAGCCCGTGCAGCTGCCCGGCGCCTAC
AACGTCAACATCAAGTTGGACATCACCTCCCACAACGAGGACTACACCATCGTGG
AACAGTACGAACGCGCCGAGGGCCGCCACTCCACCGGCGGCATGGACGAGCTGTA
CAAGTAATGAATTCGCTTATCGATAATCAACCTCTGGATTACAAAATTTGTGAAA
GATTGACTGGTATTCTTAACTATGTTGCTCCTTTTACGCTATGTGGATACGCTGC
TTTAATGCCTTTGTATCATGCTATTGCTTCCCGTATGGCTTTCATTTTCTCCTCC
TTGTATAAATCCTGGTTGCTGTCTCTTTATGAGGAGTTGTGGCCCGTTGTCAGGC
AACGTGGCGTGGTGTGCACTGTGTTTGCTGACGCAACCCCCACTGGTTGGGGCAT
TGCCACCACCTGTCAGCTCCTTTCCGGGACTTTCGCTTTCCCCCTCCCTATTGCC
ACGGCGGAACTCATCGCCGCCTGCCTTGCCCGCTGCTGGACAGGGGCTCGGCTGT
TGGGCACTGACAATTCCGTGGTGTTGTCGGGGAAATCATCGTCCTTTCCTTGGCT
GCTCGCCTGTGTTGCCACCTGGATTCTGCGCGGGACGTCCTTCTGCTACGTCCCT
TCGGCCCTCAATCCAGCGGACCTTCCTTCCCGCGGCCTGCTGCCGGCTCTGCGGC
CTCTTCCGCGTCTTCGCCTTCGCCCTCAGACGAGTCGGATCTCCCTTTGGGCCGC
CTCCCCGCATCGATACCGAGCGCTGCTCGAGAGATCTACGGGTGGCATCCCTGTG
ACCCCTCCCCAGTGCCTCTCCTGGCCCTGGAAGTTGCCACTCCAGTGCCCACCAG
CCTTGTCCTAATAAAATTAAGTTGCATCATTTTGTCTGACTAGGTGTCCTTCTAT
AATATTATGGGGTGGAGGGGGGTGGTATGGAGCAAGGGGCAAGTTGGGAAGACAA
CCTGTAGGGCCTGCGGGGTCTATTGGGAACCAAGCTGGAGTGCAGTGGCACAATC
TTGGCTCACTGCAATCTCCGCCTCCTGGGTTCAAGCGATTCTCCTGCCTCAGCCT
CCCGAGTTGTTGGGATTCCAGGCATGCATGACCAGGCTCAGCTAATTTTTGTTTT
ITTGGTAGAGACGGGGTTTCACCATATTGGCCAGGCTGGTCTCCAACTCCTAATC
TCAGGTGATCTACCCACCTTGGCCTCCCAAATTGCTGGGATTACAGGCGTGAACC
ACTGCTCCCTTCCCTGTCCTTCTGATTTTGTAGGTAACCACGTGCGGACCGAGCG
GCCGCAGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCGG
CCTCAGTGAGCGAGCGAGCGCGCAGCTGCCTGCAGGGGCGCCTGATGCGGTATTT
TCTCCTTACGCATCTGTGCGGTATTTCACACCGCATACGTCAAAGCAACCATAGT
ACGCGCCCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTCCCTTCC
TTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTTGGG
TGATGGTTCACGTAGTGGGCCATCGCCCTGATAGACGGTTTTTCGCCCTTTGACG
TTGGAGTCCACGTTCTTTAATAGTGGACTCTTGTTCCAAACTGGAACAACACTCA
ACCCTATCTCGGGCTATTCTTTTGATTTATAAGGGATTTTGCCGATTTCGGCCTA
TTGGTTAAAAAATGAGCTGATTTAACAAAAATTTAACGCGAATTTTAACAAAATA
TTAACGTTTACAATTTTATGGTGCACTCTCAGTACAATCTGCTCTGATGCCGCAT
AGTTAAGCCAGCCCCGACACCCGCCAACACCCGCTGACGCGCCCTGACGGGCTTG
TCTGCTCCCGGCATCCGCTTACAGACAAGCTGTGACCGTCTCCGGGAGCTGCATG
TGTCAGAGGTTTTCACCGTCATCACCGAAACGCGCGAGACGAAAGGGCCTCGTGA
TACGCCTATTTTTATAGGTTAATGTCATGATAATAATGGTTTCTTAGACGTCAGG
TGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAAT
ATTGAAAAAGGAAGAGTATGAGTATTCAACATTTCCGTGTCGCCCTTATTCCCTT
TTTTGCGGCATTTTGCCTTCCTGTTTTTGCTCACCCAGAAACGCTGGTGAAAGTA
AAAGATGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATGATGAG
CACTTTTAAAGTTCTGCTATGTGGCGCGGTATTATCCCGTATTGACGCCGGGCAA
GAGCAACTCGGTCGCCGCATACACTATTCTCAGAATGACTTGGTTGAGTACTCAC
CAGTCACAGAAAAGCATCTTACGGATGGCATGACAGTAAGAGAATTATGCAGTGC
TGCCATAACCATGAGTGATAACACTGCGGCCAACTTACTTCTGACAACGATCGGA
GGACCGAAGGAGCTAACCGCTTTTTTGCACAACATGGGGGATCATGTAACTCGCC
TTGATCGTTGGGAACCGGAGCTGAATGAAGCCATACCAAACGACGAGCGTGACAC
CACGATGCCTGTAGCAATGGCAACAACGTTGCGCAAACTATTAACTGGCGAACTA
CTTACTCTAGCTTCCCGGCAACAATTAATAGACTGGATGGAGGCGGATAAAGTTG
CAGGACCACTTCTGCGCTCGGCCCTTCCGGCTGGCTGGTTTATTGCTGATAAATC
TGGAGCCGGTGAGCGTGGGTCTCGCGGTATCATTGCAGCACTGGGGCCAGATGGT
AAGCCCTCCCGTATCGTAGTTATCTACACGACGGGGAGTCAGGCAACTATGGATG
AACGAAATAGACAGATCGCTGAGATAGGTGCCTCACTGATTAAGCATTGGTAACT
GTCAGACCAAGTTTACTCATATATACTTTAGATTGATTTAAAACTTCATTTTTAA
TTTAAAAGGATCTAGGTGAAGATCCTTTTTGATAATCTCATGACCAAAATCCCTT
AACGTGAGTTTTCGTTCCACTGAGCGTCAGACCCCGTAGAAAAGATCAAAGGATC
TTCTTGAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTTTCCGA
AGGTAACTGGCTTCAGCAGAGCGCAGATACCAAATACTGTCCTTCTAGTGTAGCC
GTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGT
TGGACTCAAGACGATAGTTACCGGATAAGGCGCAGCGGTCGGGCTGAACGGGGGG
TTCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAGGT
ATCCGGTAAGCGGCAGGGTCGGAACAGGAGAGCGCACGAGGGAGCTTCCAGGGGG
AAACGCCTGGTATCTTTATAGTCCTGTCGGGTTTCGCCACCTCTGACTTGAGCGT
CGATTTTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGT

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations that is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating a neurological disease, disorder, or injury associated with upper motor neuron activity in a subject in need thereof, the method comprising administering to the subject a therapeutic agent that results in an increase in the concentration of ubiquitin carboxy-terminal hydrolase ligase 1 (UCHL1) in upper motor neurons of the subject relative to the concentration of UCHL1 in the upper motor neurons of the subject prior to administering the therapeutic agent.

2. The method of claim 1, wherein the subject has amyotrophic lateral sclerosis (ALS).

3. The method of claim 1, wherein the subject has hereditary spastic paraplegia (HSP).

4. The method of claim 1, wherein the subject has primary lateral sclerosis (PLS).

5. The method of claim 1, wherein the subject has a spinal cord injury.

6. The method of claim 1, wherein the therapeutic agent is administered to the motor neurons of the subject.

7. The method of claim 1, wherein the therapeutic agent is a vector that expresses UCHL1 or a variant thereof comprising an amino acid sequence having at least about 80% sequence identity to any of SEQ ID NOs:1-6.

8. The method of claim 1, wherein the therapeutic agent is a viral vector that expresses UCHL1 or a variant thereof comprising an amino acid sequence having at least about 80% sequence identity to any of SEQ ID NOs:1-6.

9. The method of claim 1, wherein the therapeutic agent is an adenovirus-associated viral (AAV) vector that expresses UCHL1 or a variant thereof comprising an amino acid sequence having at least about 80% sequence identity to any of SEQ ID NOs:1-6.

10. The method of claim 1, wherein the therapeutic agent is an adenovirus-associated viral (AAV) vector serotype 1, 2, 3, 4, 5, 6, 7, 8, or 9 that expresses UCHL1 or a variant thereof comprising an amino acid sequence having at least about 80% sequence identity to any of SEQ ID NOs:1-6.

11. The method of claim 1, wherein the therapeutic agent is an AAV2 vector that expresses UCHL1 or a variant thereof comprising an amino acid sequence having at least about 80% sequence identity to any of SEQ ID NOs:1-6.

12. The method of claim 1, wherein the therapeutic agent is a vector that expresses UCHL1 or a variant thereof comprising an amino acid sequence having at least about 80% sequence identity to any of SEQ ID NOs:1-6 utilizing a promoter selected from the group consisting of CMV promoter (i.e., strong mammalian expression promoter from the human cytomegalovirus), CBA promoter (i.e., chicken β-actin promoter), UCHL1 promoter (i.e., promoter for ubiquitin carboxy-terminal hydrolase ligase 1 gene), EF1α promoter (i.e., strong mammalian expression from human elongation factor 1 α), SV40 promoter (i.e., mammalian expression promoter from the simian vacuolating virus 40), PGK1 promoter (i.e., mammalian promoter from phosphoglycerate kinase gene), Ubc promoter (i.e., mammalian promoter from the human ubiquitin C gene), and human beta actin promoter (i.e., mammalian promoter from beta actin gene).

13. The method of claim 1, wherein the therapeutic agent is a vector that expresses UCHL1 or a variant thereof comprising an amino acid sequence having at least about 80% sequence identity to any of SEQ ID NOs:1-6 utilizing the promoter for the human HCHL1 gene.

14. A vector that is capable of expressing a therapeutic gene product in upper motor neurons of a subject in need thereof, the vector comprising a UCHL1 promoter operably linked to a nucleic acid sequence encoding the therapeutic gene product.

15. The vector of claim 14, wherein the UCHL1 promoter comprises SEQ ID NO: 16.

16. The vector of claim 14, wherein the vector is a viral vector.

17. The vector of claim 14, wherein the vector is an adenovirus-associated viral (AAV) vector.

18. The vector of claim 14, wherein the vector is adenovirus-associated viral (AAV) vector serotype 2.

19. The vector of claim 14, wherein the therapeutic gene product comprises UCHL1 or a variant thereof comprising an amino acid sequence having at least about 80% sequence identity to any of SEQ ID NOs:1-6.

20. A pharmaceutical composition comprising: (i) the vector of claim 14; and (ii) a carrier, excipient, or diluent.


SEQUENCE LISTING (17 sequences)

SEQ ID NO: 1 - protein, 223 aa, Homo sapiens [sequence omitted]

SEQ ID NO: 2 - protein, 142 aa, Homo sapiens
MKQTIGNSCGTIGLIHAVANNQDKLGFEDGSVLKQFLSETEKMSPEDRAKCFEKNEAIQAAHDAVAQEGQCRVDDKVNFHFILFNNVDGHLYELDGRMPFPVNHGASSEDTLLKDAAKVCREFTEREQGEVRFSAVALCKAA

SEQ ID NO: 3 - protein, 126 aa, Homo sapiens
MKQTIGNSCGTIGLIHAVANNQDKLGFEDGSVLKQFLSETEKMSPEDRAKCFEKNEVDDKVNFHFILFNNVDGHLYELDGRMPFPVNHGASSEDTLLKDAAKVCREFTEREQGEVRFSAVALCKAA

SEQ ID NO: 4 - protein, 221 aa, Homo sapiens
MQLKPMEINPEVSVLSRLGVAGQWRFVDVLGLEEESLGSVPAPACALLLLFPLTAQHENFRKKQIEELKGQEVSPKVYFMKQTIGNSCGTIGLIHAVANNQDKLGFEDGSVLKQFLSETEKMSPEDRAKCFEKNEAIQAAHDAVAQEGQCRVDDKVNFHFILFNNVDGHLYELDGRMPFPVNHGASSEDTLLKDAAKVCREFTEREQGEVRFSAVALCKAA

SEQ ID NO: 5 - protein, 403 aa, Homo sapiens
GSASCFSSSLGYFCRALREDAAQADGDQPRDAEQSEWRLAPSLAPSPASAEAGAPTGSGCWQGPSRPLRAPETAGLGRGLGAFPGPLHLAGDSTKPVTGRRRGLRPVASERQAELPRAWRGQHRLGCTGFAGATCGPRFVLCHCAGPGGGGRAGLGLLPGSGAGAEGARLLAPPPGRCPRPACPRAPGRLVSSPQVLSRLGVAGQWRFVDVLGLEEESLGSVPAPACALLLLFPLTAQHENFRKKQIEELKGQEVSPKVYFMKQTIGNSCGTIGLIHAVANNQDKLGFEDGSVLKQFLSETEKMSPEDRAKCFEKNEAIQAAHDAVAQEGQCRVDDKVNFHFILFNNVDGHLYELDGRMPFPVNHGASSEDTLLKDAAKVCREFTEREQGEVRFSAVALCKAA

SEQ ID NO: 6 - protein, 223 aa, Mus musculus
MQLKPMEINPEMLNKVLAKLGVAGQWRFADVLGLEEETLGSVPSPACALLLLFPLTAQHENFRKKQIEELKGQEVSPKVYFMKQTIGNSCGTIGLIHAVANNQDKLEFEDGSVLKQFLSETEKLSPEDRAKCFEKNEAIQAAHDSVAQEGQCRVDDKVNFHFILFNNVDGHLYELDGRMPFPVNHGASSEDSLLQDAAKVCREFTEREQGEVRFSAVALCKAA

SEQ ID NO: 7 - DNA, 7509 bp, Artificial Sequence
  Synthetic - pAAV-hUCHL1P-UCHL1-IRES-mCherry
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactagttggagcccagtttagcagggtttacttctcagttttacttttcaattgtgaccgtaagcaaattaacttctctaggcctgggattctgatctgtaaaattgcactaatatgagtctcttcacggctcctctaaggattaaatgagagacacatgcaaaggatccccaaaaacaataactcaaaaaatgttgattccctcccttccctctgtcatctgttaacctcaacttcctaaatagaaggtctattcttttaccatcatcattattctcttcggtgcctatttttaaaaaatactcaaccttcttgcttccttcgctacctaagtatttctgcaagcccactttgttctgcagcttagctttcctggcacaattcttatagattttggtcccttttaaaattcattcttcagcaaatgctttctctctccatcttttgactagagatcattagagatcacctgagatcattagagaacagtggtttccttggttgccattccctttcttcttcattgggagtattctgcggtgaactcagacattttatttttcaaagcttcccattcttttaaaaatgcttttccttttacagcctctcgctcaaaatcatacccatcttttccctggatctgttttctcaagtctccaatcgcctgccttctttgtgtcttgtattaccctcacatcccccagctttctactgctctcccaggaccaaccatttcttccgcgggagtcacattacatcagcattcctaatgcagtatctgttatctaccagattctgttttattctaggtagtcacttaaaaacgaacctcggtactggtctgacttaacatggaggaggaattgtctaaggttaaacgcaaactgctgagagatttggggcggggggcacacatttacattcattcgtattaaatatatacctgttgaatttgtgctttttctcaaatgcttcagagactcgagctttagagtaattgggatggtgaaaggatgggtttccagaaacttcgcccaaaattaaagactccatcaaaaggactgctccatacactcaaggaacacccaccaacaaatcccgtctccacaaccaccagattatctcaccggcgagtgagactgcaaggtttgggggcccggccgtaccactccgcgctgcgcacggggggttcgtacccatctggccgcgaccgtccgtttccccctcgcttggttctgcccctgctccccctgcacaggcctcacagtgcgtctggccggcgctttatagctgcagcctgggcgaccggatcacaagtttgtacaaaaaagcaggctccgcggccgcccccttcaccatgcagctgaagccgatggagattaaccccgagatgctgaacaaagtgttggccaagctgggggtcgccggccagtggcgcttcgccgacgtgctagggctggaggaggagactctgggctcagtgccatcccctgcctgcgccctgctgctcctgtttcccctcacggcccagcatgaaaacttcaggaaaaagcaaattgaggaactgaagggacaggaagttagccctaaagtttacttcatgaagcagaccatcggaaactcctgtggtaccatcgggttgatccacgcagtggccaacaaccaagacaagctggaatttgaggatggatccgtcctgaaacagtttctgtctgaaacggagaagctgtcccccgaagatagagccaagtgtttcgagaagaacgaggccatccaggcagcccatgactccgtggcccaggagggccagtgtcgggtagatgacaaagtgaatttccattttattctgttcaacaacgtggacggccatctgtacgagctcgatgggcgaatgccctttccagtgaaccatggcgccagctcagaggactctctgctgcaggatgctgccaaggtctgcagagaattcactgagcgcgagcagggggaggtccgcttctctgccgtggctctctgcaaagcagcttaaatcagatccaagggtgggcgcgccgacccagctttcttgtacaaagtggtgatccggtgctagcctcgagaattcacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaatgaattcgcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 8848 bp, Artificial Sequence
  Synthetic -pAAV-CBA-UCHL1-IRES-mCherry
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgaatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatctagcctcgagaattcacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaatgaattcgcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 9 - DNA, 22 bp, Artificial Sequence
  Synthetic - Primer used to genotype Uchl1nm3419 mice
tggacggctgtgtgtgctaatg

SEQ ID NO: 10 - DNA, 23 bp, Artificial Sequence
  Synthetic - Primer used to genotype Uchl1nm3419 mice
ctaagggaagggtcttgctcatc

SEQ ID NO: 11 - DNA, 24 bp, Artificial Sequence
  Synthetic - Primer used to genotype Uchl1nm3419 mice
gtcatctacctgaagagagccaag

SEQ ID NO: 12 - DNA, 24 bp, Artificial Sequence
  Synthetic - Primer used to genotype floxed UCHL1 mice
tagtccaatccttgtaccagttgg

SEQ ID NO: 13 - DNA, 26 bp, Artificial Sequence
  Synthetic - Primer used to genotype floxed UCHL1 mice
ccatggttctagatgctgttgaatgc

SEQ ID NO: 14 - DNA, 27 bp, Artificial Sequence
  Synthetic - Primer used to genotype cre mice
gcattaccggtcgatgcaacgagtgat

SEQ ID NO: 15 - DNA, 27 bp, Artificial Sequence
  Synthetic - Primer used to genotype cre mice
gagtgaacgaacctggtcgaaatcagt

SEQ ID NO: 16 - DNA, 1298 bp, Homo sapiens
tggagcccagtttagcagggtttacttctcagttttacttttcaattgtgaccgtaagcaaattaacttctctaggcctgggattctgatctgtaaaattgcactaatatgagtctcttcacggctcctctaaggattaaatgagagacacatgcaaaggatccccaaaaacaataactcaaaaaatgttgattccctcccttccctctgtcatctgttaacctcaacttcctaaatagaaggtctattcttttaccatcatcattattctcttcggtgcctatttttaaaaaatactcaaccttcttgcttccttcgctacctaagtatttctgcaagcccactttgttctgcagcttagctttcctggcacaattcttatagattttggtcccttttaaaattcattcttcagcaaatgctttctctctccatcttttgactagagatcattagagatcacctgagatcattagagaacagtggtttccttggttgccattccctttcttcttcattgggagtattctgcggtgaactcagacattttatttttcaaagcttcccattcttttaaaaatgcttttccttttacagcctctcgctcaaaatcatacccatcttttccctggatctgttttctcaagtctccaatcgcctgccttctttgtgtcttgtattaccctcacatcccccagctttctactgctctcccaggaccaaccatttcttccgcgggagtcacattacatcagcattcctaatgcagtatctgttatctaccagattctgttttattctaggtagtcacttaaaaacgaacctcggtactggtctgacttaacatggaggaggaattgtctaaggttaaacgcaaactgctgagagatttggggcggggggcacacatttacattcattcgtattaaatatatacctgttgaatttgtgctttttctcaaatgcttcagagactcgagctttagagtaattgggatggtgaaaggatgggtttccagaaacttcgcccaaaattaaagactccatcaaaaggactgctccatacactcaaggaacacccaccaacaaatcccgtctccacaaccaccagattatctcaccggcgagtgagactgcaaggtttgggggcccggccgtaccactccgcgctgcgcacggggggttcgtacccatctggccgcgaccgtccgtttccccctcgcttggttctgcccctgctccccctgcacaggcctcacagtgcgtctggccggcgctttatagctgcagcctgggcg

SEQ ID NO: 17 - DNA, 1666 bp, Mus musculus
cccaagctcagtgaccagtgggagtaaatgcttgcttgtgcagtatttgtgatgaatggataaatgaagaaaactggaaatgtagtatgtctctgccaaaagacagacatgcacacagtctacccaggtgtactgggttatgtgtccgtctgtcttgtattttttttttttattaactgcattgagatgtaatttacatagtataaaattcactcagaatcttactaatgccttcttgcctctcacacttggtaagtgaaggtcatttttttttccctgatggtgaaactgggaagaaaattgaagtagcacattgctgagtctaaaacagaaaatggacccttccagcctcatttaccaggttttgtcttcctgcctcattttccagctgtagcaccaagcaaatgaacatctctgaaccctggatcctgatgtataaaatttcacaaatacaatattggaccctctcagggtgaaataagatggcctggtaaaggatccagctggagaggcagtgctgttaaaacaaggttggttccctccatcctatgccttgcttacctcgggctagcaaaaagcatgtctgtttttcaccatttgttctgagcatattttttaaaatacccaacctgcctgcatccttagctacctcattctgtgatttagcatccctgacaattgatacagatctggcccctccttctactccgccttcagcacaagcgtttgccttccatcctttgaaatgtgcctgttagaaagaacacccccgcccccatccctggagaaccgcgggctctcctccgttgctattccctttcttctgcgcccaaagtattgtggaccaaaatcttattatcccaaacccctgttctttgaaaaaaggtttcccctattttttttttaaatgctttttcctttaagcctctagtctcaaatcatctatcttttctttggtctgtttctaaagtcctccggcgcccgtattctatgtgtctcgcatgtactgcttcccagcgacccaccggttcttcccctggagtcacattacatcagcttcactaatgcactccgtatcagatgttttattctaggcagtccattaaaaaccgagcctggacacaggttgaatttaacatggaggacagattgtctaaggttaagtacaaactgttccgagatgggcggggggggggagggaggaagaccgacaaatatttatattcatattaaacactacatatacctgttgattttgtgcttttttttctctctctctcaatgtttcagagaccctcgagctttggagtgattgagacgacagaggagggtttccagaaatttcgctccaaattgaaagctccctcaaataggctgcttcatgcgctcaaggaacacccaccaacaaaacccatctccacaaccaccagattagctcaccggcgagtgagactgcaaggtttgggggcttggcttgtaccattctgtgcggtgcatgggggagttcgagccctcttggccttcctcctctgcttgtttctgctcccgtctcccctgctcaggctttcccagtgagcgaggccggcgctttataacagcagcctgggcggctccaccggctgttttttcggctcctcgggtttgtgtctgcaggtgccatccgcga